(19) United States
(12) Patent Application Publication
(10) Pub. No.: US 2022/0388953 A1
(43) Pub. Date: Dec. 8, 2022

(54) COMPOUNDS WITH ANTIMICROBIAL ACTIVITY

(71) Applicant: The Hong Kong Polytechnic University, Hong Kong (CN)

(72) Inventor: Cong MA, Hong Kong (CN)

(21) Appl. No.: 17/250,178

(22) PCT Filed: Jun. 18, 2019
(86) PCT No.: PCT/CN2019/091670

§ 371 Date: Dec. 9, 2020

Related U.S. Application Data
(60) Provisional application No. 62/690,470, filed on Jun. 27, 2018.

(51) Int. Cl. C07C 323/33; A61P 31/04; C07D 213/89; C07C 225/22; C07C 233/65; C07D 215/40; C07D 295/155; C07D 209/50; C07D 277/587; C07D 307/64

(52) U.S. Cl. CPC C07C 323/33; A61P 31/04; C07D 213/89; C07C 225/22; C07C 233/65; C07D 215/40; C07D 295/155; C07D 209/50; C07D 277/587; C07D 307/64; C07C 2601/16

(57) ABSTRACT

Provided herein are compounds useful in the treatment of bacterial infections, pharmaceuticals comprising the same, and methods of use and preparation thereof.

### CROSS-REFERENCE TO RELATED APPLICATIONS

This application claims the benefit of priority of U.S. Provisional Application No. 62/690,470, filed on Jun. 27, 2018, the contents of which being hereby incorporated by reference in their entirety for all purposes.

### TECHNICAL FIELD

The present disclosure generally relates to the field of antibacterial agents. More particularly the present disclosure provides compositions having antibacterial properties, pharmaceutical compositions comprising the same, and methods of preparation and use thereof.

### BACKGROUND

Bacterial infection is a global health problem responsible for numerous deadly and debilitating diseases such as pneumonia, tuberculosis, and many gastrointestinal diseases including peptic ulcers, which cost health systems billions of dollars annually worldwide. They are projected to cause >10 million deaths per year (more than the current annual number of deaths due to cancer) and cost the global economy U.S. $100 trillion per year by 2050 if no new efficient drugs are developed.

Transcription, together with DNA replication and protein translation, is an essential step in the bacterial cell cycle, which requires RNA polymerase (RNAP) as the core enzyme along with a suite of transcription factors to convert DNA sequences into RNA containing the same genetic information. The discovery and development of rifampicin in the 1950s established bacterial transcription as a valid target for antibiotic development. Despite the subsequent identification of many further inhibitors of transcription, only fidaxomicin, in 2011, has successfully gained approval for clinical use as a narrow spectrum drug for the treatment of Clostridium difficile-associated diarrhea.

The initiation of transcription requires RNAP holoenzyme, which is formed by association of the RNAP core enzyme (α₂ββ′ω and 8 in the Gram-positive firmicutes) with an essential a factor (FIG. 1A, B; σ^{A} in Bacillus subtilis) required for specific recognition of promoter DNA sequences. Therefore, compounds that prevent holoenzyme formation should be able to inhibit bacterial transcription and could potentially be developed as antibacterials.

The interaction between α-helices in the β′ subunit clamp-helix (β′-CH) region and a region 2.2 (σ_{2.2}) that is absolutely necessary for the formation of holoenzyme. This invention provides compounds for disruption of σ_{2.2}-β′-CH interaction to result in bacterial cell proliferation.

### SUMMARY

Provided herein are compounds that exhibit antimicrobial activity. In a first aspect, provided herein is a compound of Formula 1:

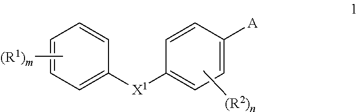

or a pharmaceutically acceptable salt, solvate, or tautomer thereof, wherein

A represents a moiety selected from the group consisting of:

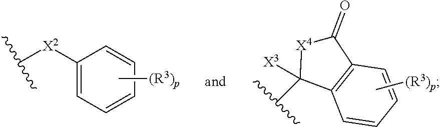

m is a whole number selected from 1-5;

n is a whole number selected from 1-4;

p is a whole number selected from 1-4;

q is a whole number selected from 2-5;

X¹ is —S—, —O—, —C(R)₂—, or —N(R)—;

X² is —S—, —O—, —C(R)₂—, —N(R)—, —C(O)—, —C(S)—, —C(O)C(R)₂—*, —C(R)₂C(O)—*, —N═CH—*, —(CR₂)_{q}—, —CH═N—*, —(R)C═C(R)—*, —C≡C*, —NRC(R)₂—*, —CR₂N(R)—*, —OC(R)₂—*, —C(R)₂—O—*, —SC(R)₂—*, —C(R)₂S—*, —N(R)C(O)—*, —C(O)N(R)—*, —CR(OH)C(R)₂—*, —CR₂CR(OH)—*, —S(O)C(R)₂—*, —S(O)₂C(R)₂—*, —C(R)₂S(O)—*, —C(R)₂S(O)₂—*, —S(O)CR(OH)—*, —S(O)₂CR(OH)—*, —CR(OH)S(O)—*, —CR(OH)S(O)₂—*, —S(O)N(R)—*, —S(O)₂N(R)—*, —N(R)S(O)—*, —NRS(O)₂—*, —S(O)NR(OH)—*, —S(O)₂NR(OH)—*, —NR(OH)S(O)—*, or —NR(OH)S(O)₂—*, wherein * indicates the position of a covalent bond with the moiety:

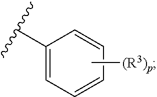

X³ is hydrogen or —OR;

X⁴ is —O—, —N(OR⁴)—, or —N(R⁴)—;

R for each instance is independently selected from the group consisting of hydrogen, alkyl, haloalkyl, alkenyl, alkynyl, cycloalkyl, heterocycloalkyl, aryl, heteroaryl, or aralkyl; or two instances of R together with the atom they are covalently bonded form a 3-6 membered cycloalkyl and heterocyloalkyl;

R¹ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;

R² for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)—N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;

R³ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; and

R⁴ is selected from the group consisting of hydrogen, alkyl, haloalkyl, alkenyl, alkynyl, cycloalkyl, heterocycloalkyl, aryl, heteroaryl, aralkyl, or —(CR₂)ₜY, wherein t is 1-10 and Y is halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkenyl, alkynyl, cycloalkyl, heterocycloalkyl, aryl, heteroaryl, and aralkyl,

with the proviso that the compound of Formula 1 does not have the structure:

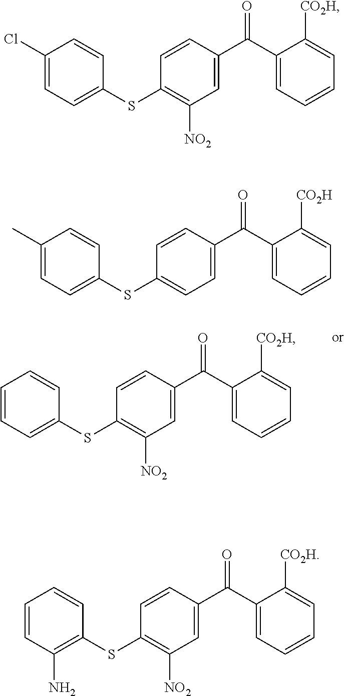

In a first embodiment of the first aspect, provided herein the compound of the first aspect, wherein X¹ is —S— or —O—.

In a second embodiment of the first aspect, provided herein the compound of the first aspect, wherein X² is —S—, —O—, —C(R)₂—, —C(O)—, —C(O)C(R)₂—*, —(R)C═C(R)—*, —CR₂N(R)—*, or —S(O)₂N(R)—*.

In a third embodiment of the first aspect, provided herein the compound of the first aspect, wherein the compound has the Formula 2:

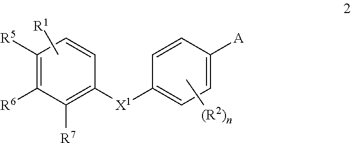

wherein each of R⁵ and R⁶ is independently selected from the group consisting of halide, —N(R)₂, —N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, and aralkyl; each of R⁵ and R⁷ is independently selected from the group consisting of halide, —N(R)₂, —N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, and aralkyl; or R⁷ is halide, —N(R)₂, N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, or aralkyl.

In a fourth embodiment of the first aspect, provided herein the compound of the first aspect, wherein A is:

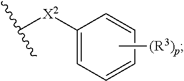

and

X² is —S—, —O—, —CH₂—, —C(O)—, —C(O)CH₂—*, —(R)C═C(R)—*, —CR₂N(R)—*, or —S(O)₂N(R)—*.

In a fifth embodiment of the first aspect, provided herein the compound of the fourth embodiment of the first aspect, wherein the compound has the Formula 3:

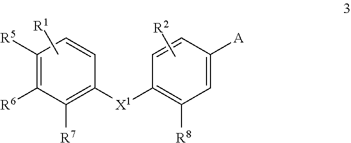

wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl; and

R⁸ is hydrogen, nitro, —N(R)₂, —N(R)C(O)R, —N(R)C(O)OR, —N(R)C(O)N(R)₂, —N(R)S(O)₂R, or perhaloalkoxyl, wherein at least one of R⁵, R⁶, and R⁷ is not hydrogen.

In a sixth embodiment of the first aspect, provided herein the compound of the fifth embodiment of the first aspect, wherein A is

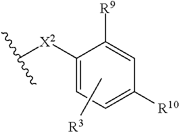

wherein each of R⁹ and R¹⁰ is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; X² is —S—, —O—, —C(R)₂—, —(R)C═C(R)—*, —CR₂N(R)—*, or —S(O)₂N(R)—*, wherein at least one of R⁹ and R¹⁰ is —C(O)OH.

In a seventh embodiment of the first aspect, provided herein the compound of the sixth embodiment of the first aspect, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

In an eighth embodiment of the first aspect, provided herein the compound of the first aspect, wherein A is:

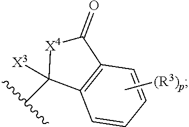

and

X³ is —OR; and X⁴ is —N(OR⁴)— or —N(R⁴)—.

In a ninth embodiment of the first aspect, provided herein the compound of the eighth embodiment of the first aspect, wherein X³ is —OH.

In a tenth embodiment of the first aspect, provided herein the compound of the ninth embodiment of the first aspect, wherein the compound has the Formula 3:

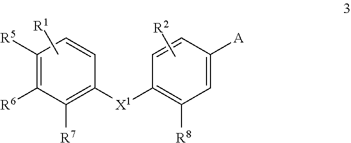

wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl; and

R⁸ is hydrogen, nitro, —N(R)₂, —N(R)C(O)R, —N(R)C(O)OR, —N(R)C(O)N(R)₂, —N(R)S(O)₂R, or perhaloalkoxyl, wherein at least one of R⁵, R⁶, and R⁷ is not hydrogen.

In an eleventh embodiment of the first aspect, provided herein the compound of the tenth embodiment of the first aspect, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

In a twelfth embodiment of the first aspect, provided herein the compound of the eleventh embodiment of the first aspect, wherein X⁴ is —N(OR⁴)— and R⁴ is hydrogen, alkyl, or cycloalkyl; or X⁴ is —N(R⁴)— and R⁴ is hydrogen, alkyl, cycloalkyl, aryl, or —(CR₂)ₜY, wherein t is 2-4 and Y is —OR, —SR, —N(R)₂, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, or —N(R)S(O)₂R.

In a thirteenth embodiment of the first aspect, provided herein the compound of the first aspect, wherein the compound of Formula 1 is selected from the group consisting of:

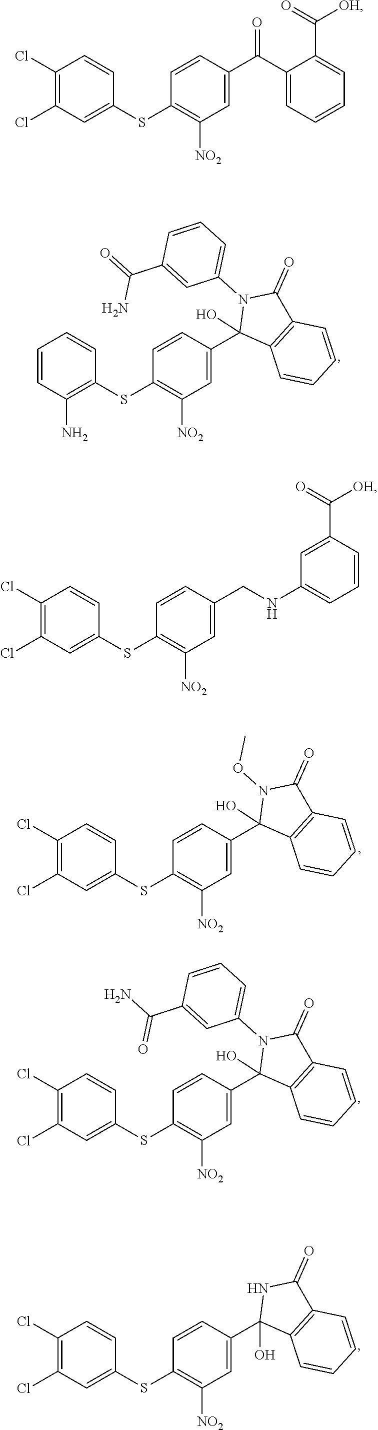

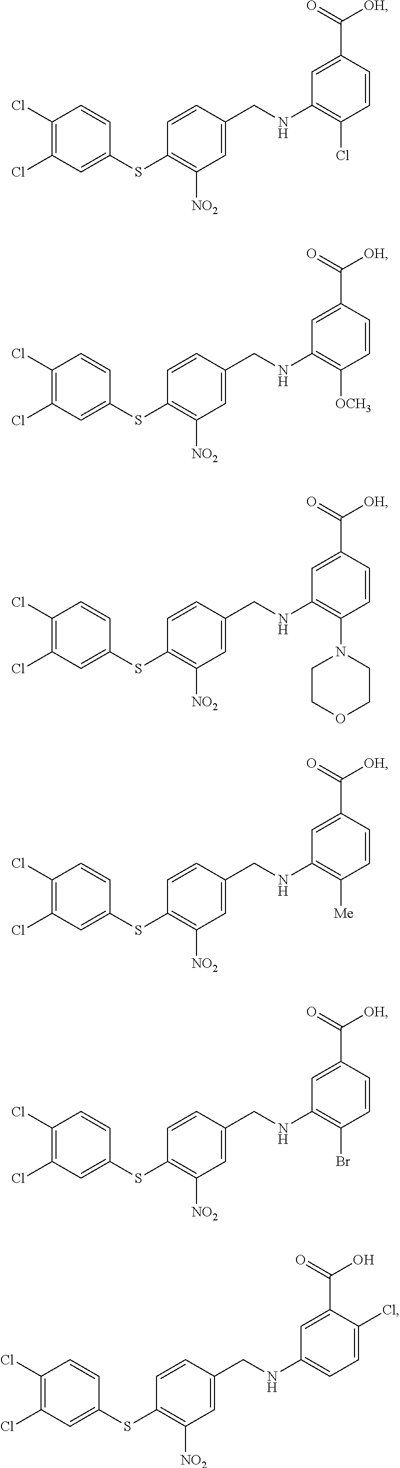 [SMILES: O=C(O)c1ccc(Cl)c(NCc2ccc(Sc3ccc(Cl)c(Cl)c3)c([N+](=O)[O-])c2)c1 ; COc1ccc(C(=O)O)cc1NCc1ccc(Sc2ccc(Cl)c(Cl)c2)c([N+](=O)[O-])c1 ; O=C(O)c1ccc(N2CCOCC2)c(NCc2ccc(Sc3ccc(Cl)c(Cl)c3)c([N+](=O)[O-])c2)c1 ; Cc1ccc(C(=O)O)cc1NCc1ccc(Sc2ccc(Cl)c(Cl)c2)c([N+](=O)[O-])c1 ; O=C(O)c1ccc(Br)c(NCc2ccc(Sc3ccc(Cl)c(Cl)c3)c([N+](=O)[O-])c2)c1 ; O=C(O)c1cc(NCc2ccc(Sc3ccc(Cl)c(Cl)c3)c([N+](=O)[O-])c2)ccc1Cl]

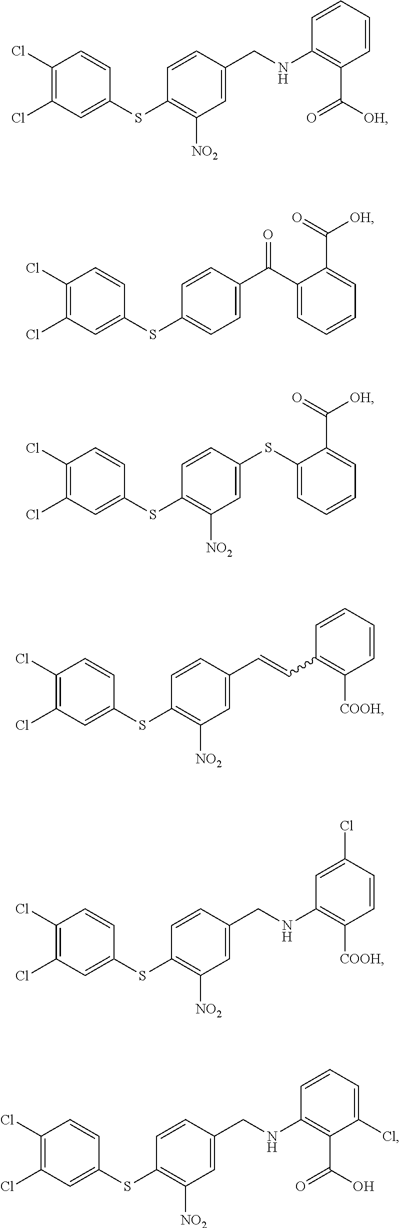 [SMILES: O=C(O)c1ccccc1NCc1ccc(Sc2ccc(Cl)c(Cl)c2)c([N+](=O)[O-])c1 ; O=C(O)c1ccccc1C(=O)c1ccc(Sc2ccc(Cl)c(Cl)c2)cc1 ; O=C(O)c1ccccc1Sc1ccc(Sc2ccc(Cl)c(Cl)c2)c([N+](=O)[O-])c1 ; O=C(O)c1ccccc1C=Cc1ccc(Sc2ccc(Cl)c(Cl)c2)c([N+](=O)[O-])c1 ; O=C(O)c1ccc(Cl)cc1NCc1ccc(Sc2ccc(Cl)c(Cl)c2)c([N+](=O)[O-])c1 ; O=C(O)c1c(Cl)cccc1NCc1ccc(Sc2ccc(Cl)c(Cl)c2)c([N+](=O)[O-])c1]

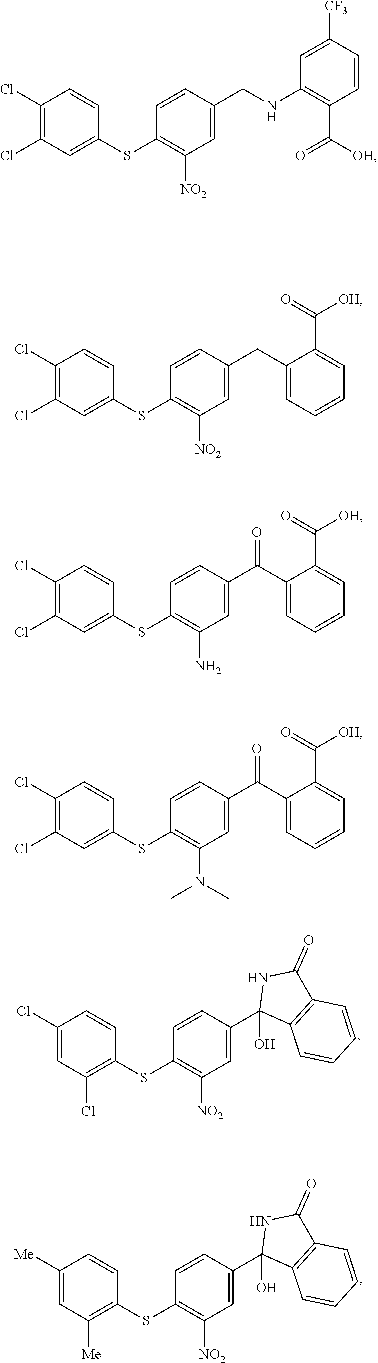 [SMILES: O=C(O)c1ccc(C(F)(F)F)cc1NCc1ccc(Sc2ccc(Cl)c(Cl)c2)c([N+](=O)[O-])c1 ; O=C(O)c1ccccc1Cc1ccc(Sc2ccc(Cl)c(Cl)c2)c([N+](=O)[O-])c1 ; Nc1cc(C(=O)c2ccccc2C(=O)O)ccc1Sc1ccc(Cl)c(Cl)c1 ; CN(C)c1cc(C(=O)c2ccccc2C(=O)O)ccc1Sc1ccc(Cl)c(Cl)c1 ; O=C1NC(O)(c2ccc(Sc3ccc(Cl)cc3Cl)c([N+](=O)[O-])c2)c2ccccc21 ; Cc1ccc(Sc2ccc(C3(O)NC(=O)c4ccccc43)cc2[N+](=O)[O-])c(C)c1]

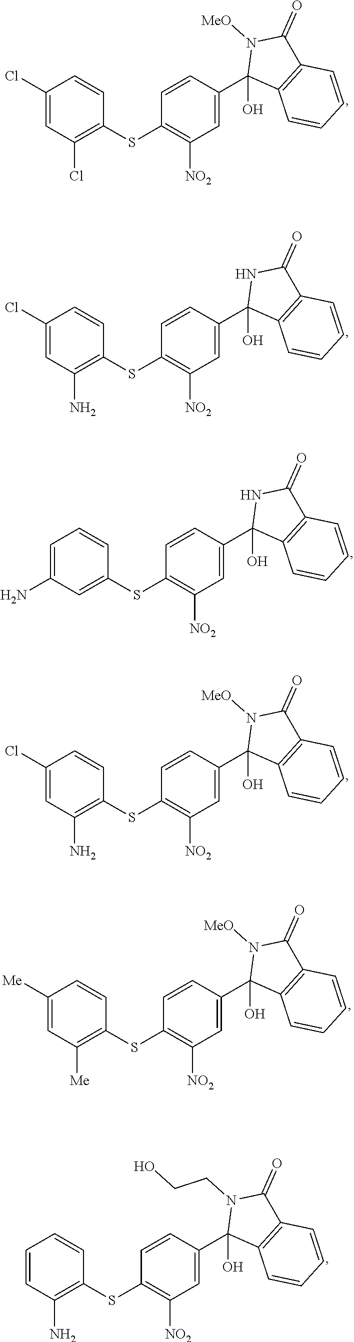 [SMILES: CON1C(=O)c2ccccc2C1(O)c1ccc(Sc2ccc(Cl)cc2Cl)c([N+](=O)[O-])c1 ; Nc1cc(Cl)ccc1Sc1ccc(C2(O)NC(=O)c3ccccc32)cc1[N+](=O)[O-] ; Nc1cccc(Sc2ccc(C3(O)NC(=O)c4ccccc43)cc2[N+](=O)[O-])c1 ; CON1C(=O)c2ccccc2C1(O)c1ccc(Sc2ccc(Cl)cc2N)c([N+](=O)[O-])c1 ; CON1C(=O)c2ccccc2C1(O)c1ccc(Sc2ccc(C)cc2C)c([N+](=O)[O-])c1 ; Nc1ccccc1Sc1ccc(C2(O)c3ccccc3C(=O)N2CCO)cc1[N+](=O)[O-]]

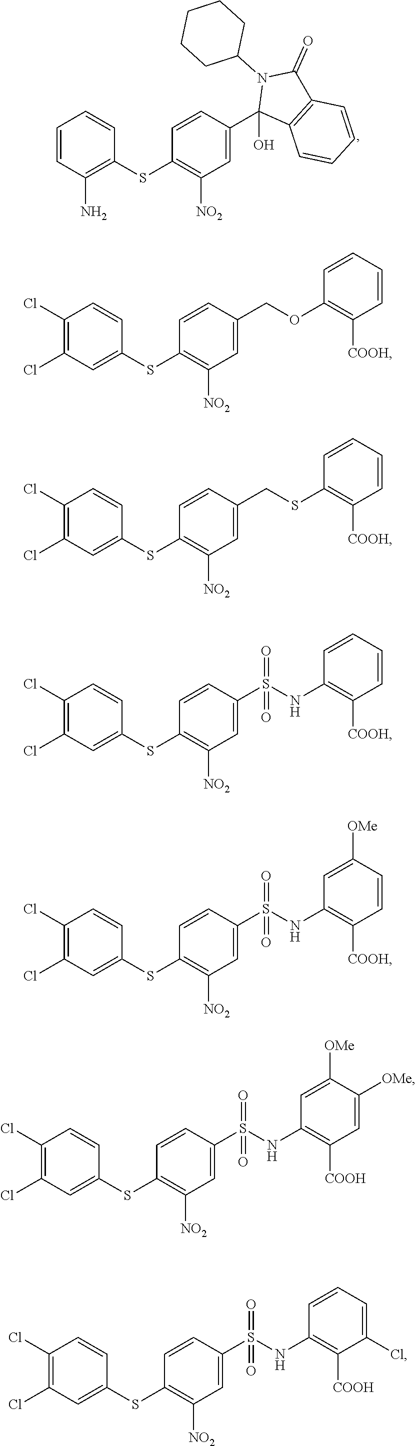

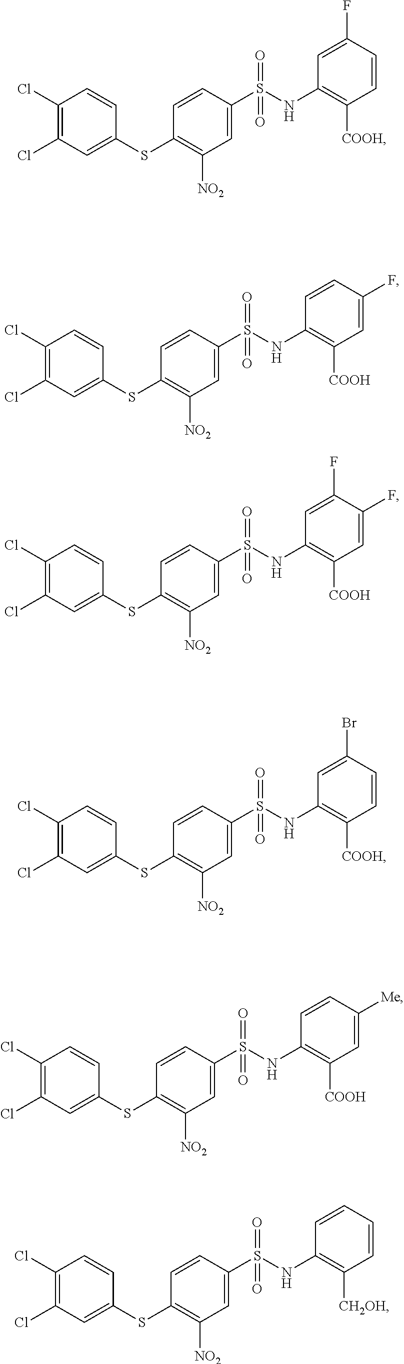 [SMILES: O=C(O)c1ccc(F)cc1NS(=O)(=O)c1ccc(Sc2ccc(Cl)c(Cl)c2)c([N+](=O)[O-])c1 ; O=C(O)c1cc(F)ccc1NS(=O)(=O)c1ccc(Sc2ccc(Cl)c(Cl)c2)c([N+](=O)[O-])c1 ; O=C(O)c1cc(F)c(F)cc1NS(=O)(=O)c1ccc(Sc2ccc(Cl)c(Cl)c2)c([N+](=O)[O-])c1 ; O=C(O)c1ccc(Br)cc1NS(=O)(=O)c1ccc(Sc2ccc(Cl)c(Cl)c2)c([N+](=O)[O-])c1 ; Cc1ccc(NS(=O)(=O)c2ccc(Sc3ccc(Cl)c(Cl)c3)c([N+](=O)[O-])c2)c(C(=O)O)c1 ; O=[N+]([O-])c1cc(S(=O)(=O)Nc2ccccc2CO)ccc1Sc1ccc(Cl)c(Cl)c1]

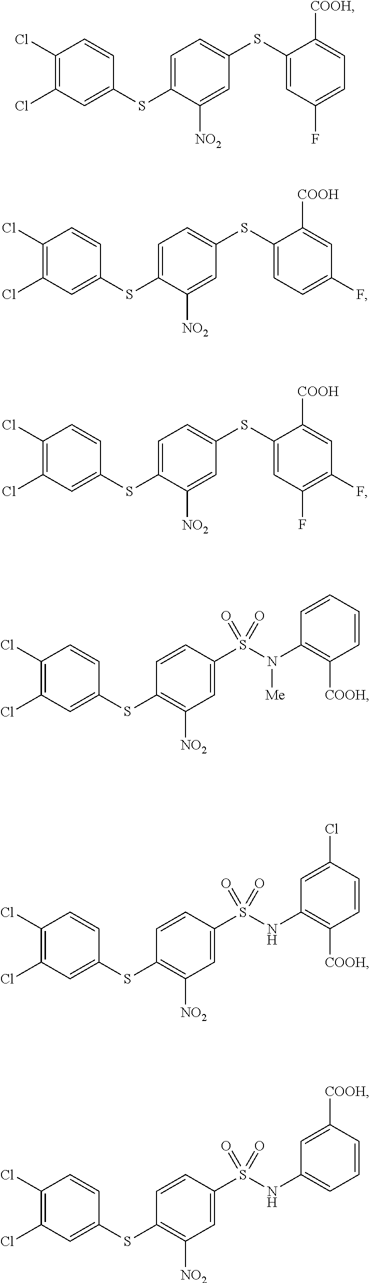 [SMILES: O=C(O)c1ccc(F)cc1Sc1ccc(Sc2ccc(Cl)c(Cl)c2)c([N+](=O)[O-])c1 ; O=C(O)c1cc(F)ccc1Sc1ccc(Sc2ccc(Cl)c(Cl)c2)c([N+](=O)[O-])c1 ; O=C(O)c1cc(F)c(F)cc1Sc1ccc(Sc2ccc(Cl)c(Cl)c2)c([N+](=O)[O-])c1 ; CN(c1ccccc1C(=O)O)S(=O)(=O)c1ccc(Sc2ccc(Cl)c(Cl)c2)c([N+](=O)[O-])c1 ; O=C(O)c1ccc(Cl)cc1NS(=O)(=O)c1ccc(Sc2ccc(Cl)c(Cl)c2)c([N+](=O)[O-])c1 ; O=C(O)c1cccc(NS(=O)(=O)c2ccc(Sc3ccc(Cl)c(Cl)c3)c([N+](=O)[O-])c2)c1]

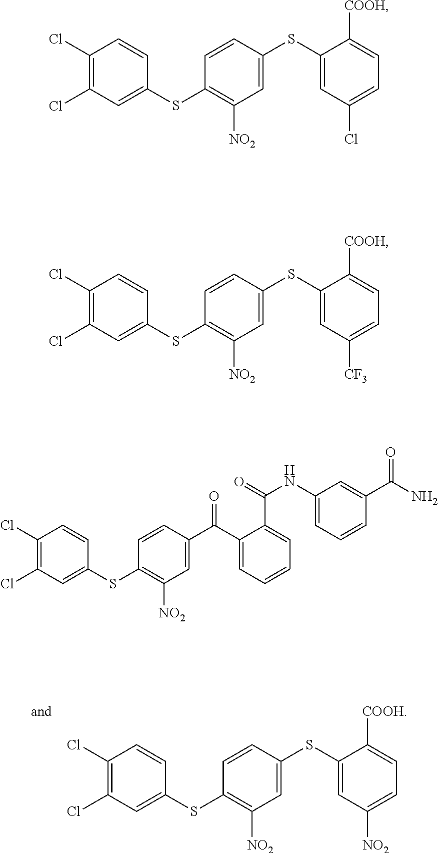 [SMILES: O=C(O)c1ccc(Cl)cc1Sc1ccc(Sc2ccc(Cl)c(Cl)c2)c([N+](=O)[O-])c1 ; O=C(O)c1ccc(C(F)(F)F)cc1Sc1ccc(Sc2ccc(Cl)c(Cl)c2)c([N+](=O)[O-])c1 ; NC(=O)c1cccc(NC(=O)c2ccccc2C(=O)c2ccc(Sc3ccc(Cl)c(Cl)c3)c([N+](=O)[O-])c2)c1 ; O=C(O)c1ccc([N+](=O)[O-])cc1Sc1ccc(Sc2ccc(Cl)c(Cl)c2)c([N+](=O)[O-])c1]

In a second aspect, provided herein is a pharmaceutical composition comprising a compound of the first aspect and at least one pharmaceutically acceptable excipient.

In a third aspect, provided herein is a method of treating a bacterial infection in a subject in need thereof comprising the step of administering a therapeutically effect amount of a compound of the first aspect to the subject.

In a first embodiment of the third aspect, provided herein is the method of the third aspect, wherein the compound has the Formula 3:

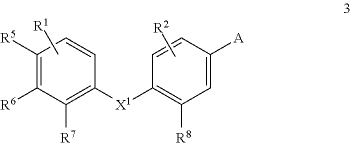

wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl;

R⁸ is hydrogen, nitro, —N(R)₂, —N(R)C(O)R, —N(R)C(O)OR, —N(R)C(O)N(R)₂, —N(R)S(O)₂R, or perhaloalkoxyl; and

A is:

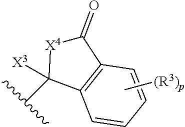

wherein X³ is —OH; and X⁴ is —N(OR⁴)— or —N(R⁴)—; or

A is:

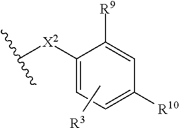

wherein X² is —S—, —O—, —CH₂—, —C(O)—, —C(O)CH₂—*, —(R)C═C(R)—*, —CR₂N(R)—*, or —S(O)₂N(R)—*; each of R⁹ and R¹⁰ is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; X² is —S—, —O—, —C(R)₂—, —(R)C═C(R)—*, —CR₂N(R)—*, or —S(O)₂N(R)—*, wherein at least one of R⁹ and R¹⁰ is —C(O)OH and at least one of R⁵, R⁶, and R⁷ is not hydrogen.

In a second embodiment of the third aspect, provided herein is the method of the first embodiment of the third aspect, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

In a third embodiment of the third aspect, provided herein is the method of the third aspect, wherein the compound is selected from the group consisting of:

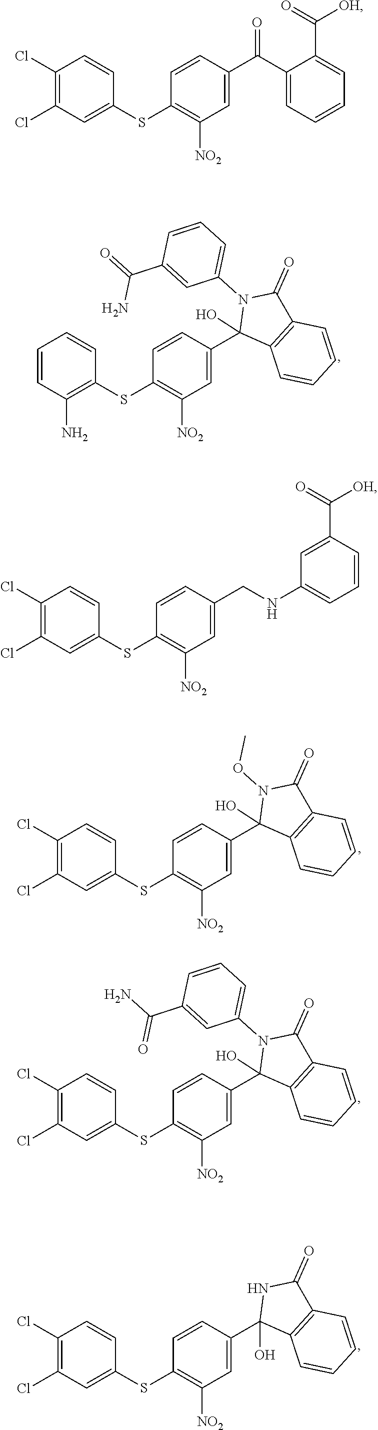

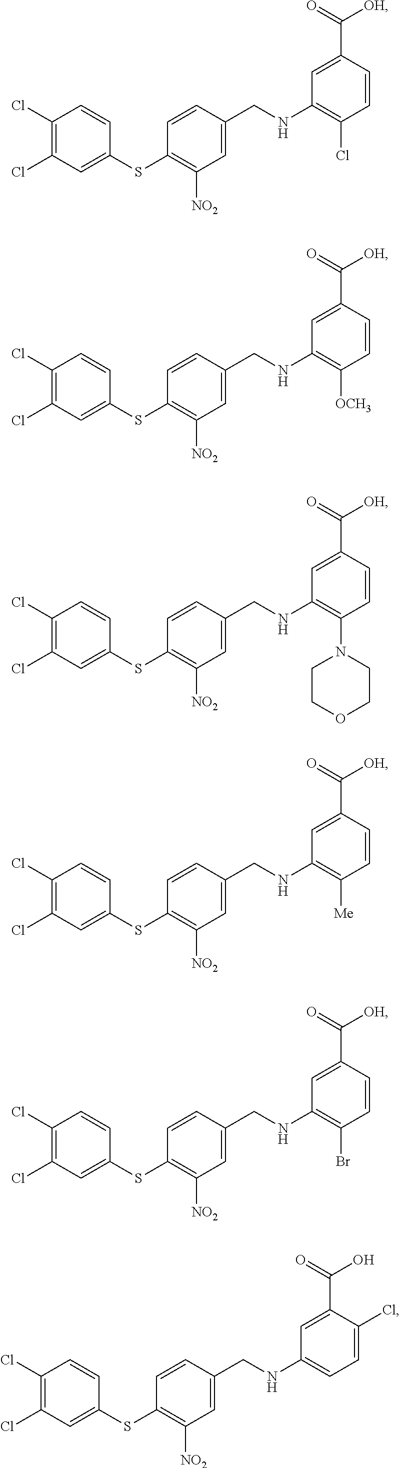 [SMILES: O=C(O)c1ccc(Cl)c(NCc2ccc(Sc3ccc(Cl)c(Cl)c3)c([N+](=O)[O-])c2)c1 ; COc1ccc(C(=O)O)cc1NCc1ccc(Sc2ccc(Cl)c(Cl)c2)c([N+](=O)[O-])c1 ; O=C(O)c1ccc(N2CCOCC2)c(NCc2ccc(Sc3ccc(Cl)c(Cl)c3)c([N+](=O)[O-])c2)c1 ; Cc1ccc(C(=O)O)cc1NCc1ccc(Sc2ccc(Cl)c(Cl)c2)c([N+](=O)[O-])c1 ; O=C(O)c1ccc(Br)c(NCc2ccc(Sc3ccc(Cl)c(Cl)c3)c([N+](=O)[O-])c2)c1 ; O=C(O)c1cc(NCc2ccc(Sc3ccc(Cl)c(Cl)c3)c([N+](=O)[O-])c2)ccc1Cl]

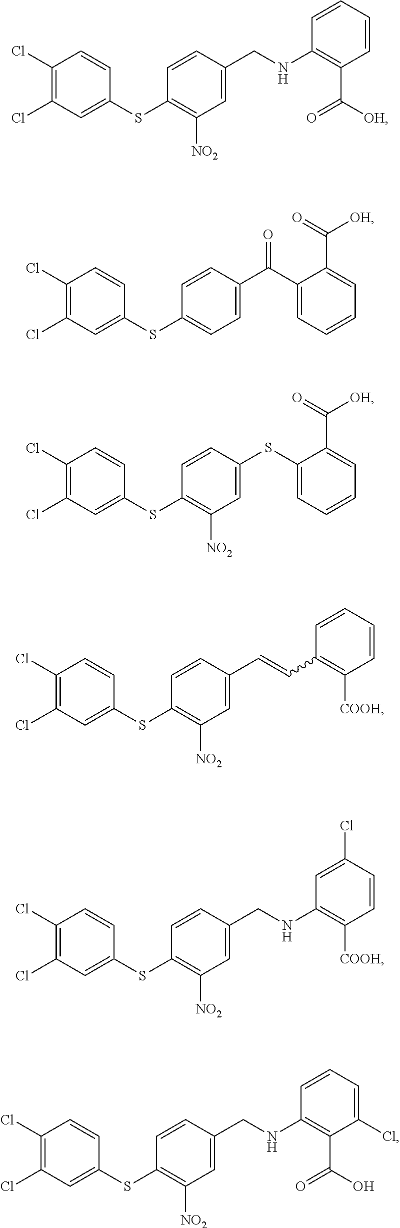 [SMILES: O=C(O)c1ccccc1NCc1ccc(Sc2ccc(Cl)c(Cl)c2)c([N+](=O)[O-])c1 ; O=C(O)c1ccccc1C(=O)c1ccc(Sc2ccc(Cl)c(Cl)c2)cc1 ; O=C(O)c1ccccc1Sc1ccc(Sc2ccc(Cl)c(Cl)c2)c([N+](=O)[O-])c1 ; O=C(O)c1ccccc1C=Cc1ccc(Sc2ccc(Cl)c(Cl)c2)c([N+](=O)[O-])c1 ; O=C(O)c1ccc(Cl)cc1NCc1ccc(Sc2ccc(Cl)c(Cl)c2)c([N+](=O)[O-])c1 ; O=C(O)c1c(Cl)cccc1NCc1ccc(Sc2ccc(Cl)c(Cl)c2)c([N+](=O)[O-])c1]

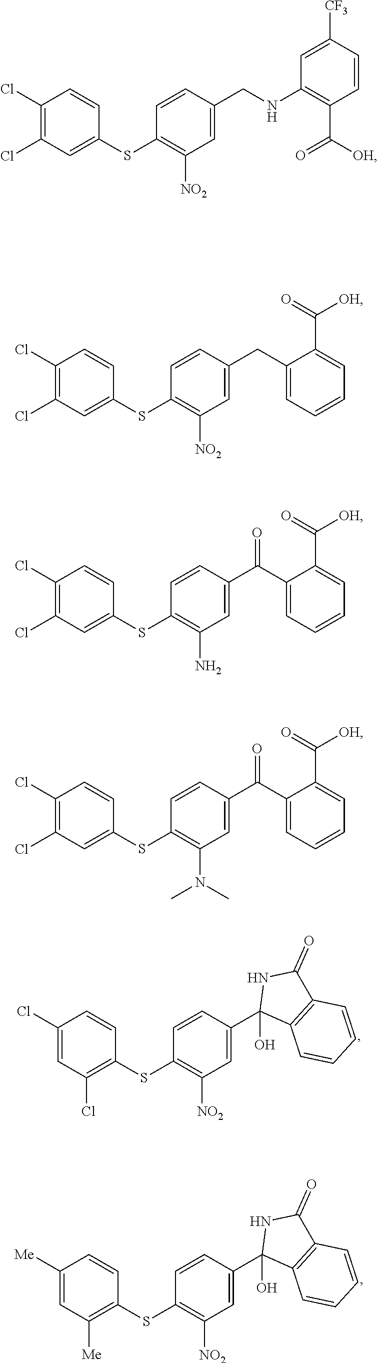 [SMILES: O=C(O)c1ccc(C(F)(F)F)cc1NCc1ccc(Sc2ccc(Cl)c(Cl)c2)c([N+](=O)[O-])c1 ; O=C(O)c1ccccc1Cc1ccc(Sc2ccc(Cl)c(Cl)c2)c([N+](=O)[O-])c1 ; Nc1cc(C(=O)c2ccccc2C(=O)O)ccc1Sc1ccc(Cl)c(Cl)c1 ; CN(C)c1cc(C(=O)c2ccccc2C(=O)O)ccc1Sc1ccc(Cl)c(Cl)c1 ; O=C1NC(O)(c2ccc(Sc3ccc(Cl)cc3Cl)c([N+](=O)[O-])c2)c2ccccc21 ; Cc1ccc(Sc2ccc(C3(O)NC(=O)c4ccccc43)cc2[N+](=O)[O-])c(C)c1]

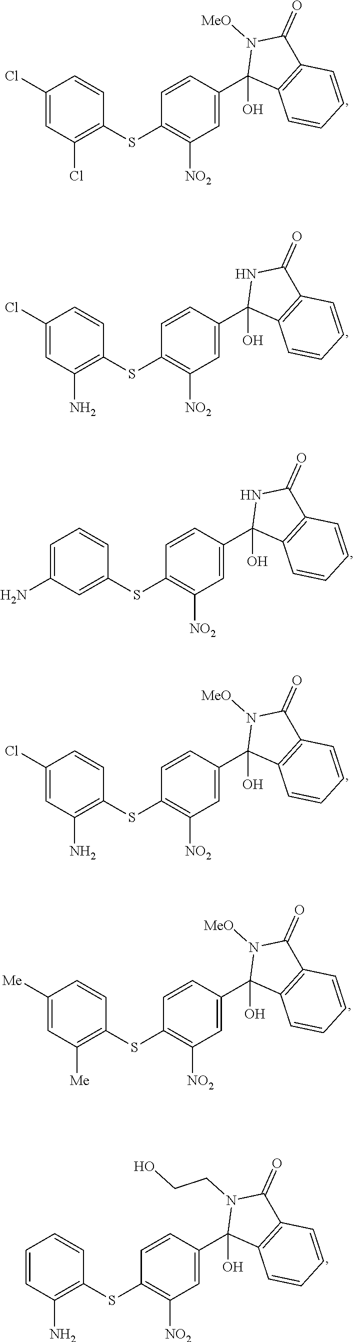 [SMILES: CON1C(=O)c2ccccc2C1(O)c1ccc(Sc2ccc(Cl)cc2Cl)c([N+](=O)[O-])c1 ; Nc1cc(Cl)ccc1Sc1ccc(C2(O)NC(=O)c3ccccc32)cc1[N+](=O)[O-] ; Nc1cccc(Sc2ccc(C3(O)NC(=O)c4ccccc43)cc2[N+](=O)[O-])c1 ; CON1C(=O)c2ccccc2C1(O)c1ccc(Sc2ccc(Cl)cc2N)c([N+](=O)[O-])c1 ; CON1C(=O)c2ccccc2C1(O)c1ccc(Sc2ccc(C)cc2C)c([N+](=O)[O-])c1 ; Nc1ccccc1Sc1ccc(C2(O)c3ccccc3C(=O)N2CCO)cc1[N+](=O)[O-]]

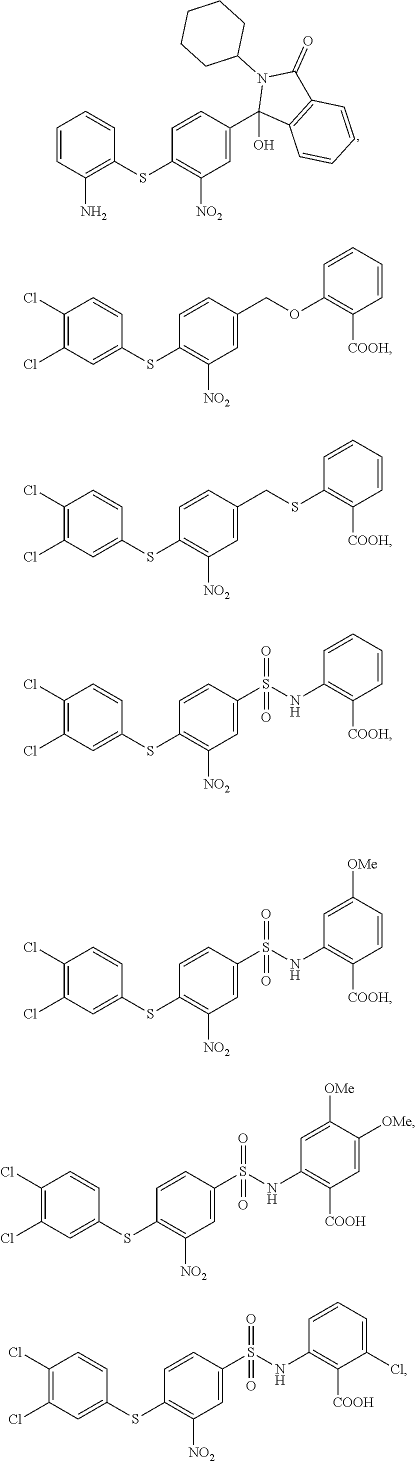

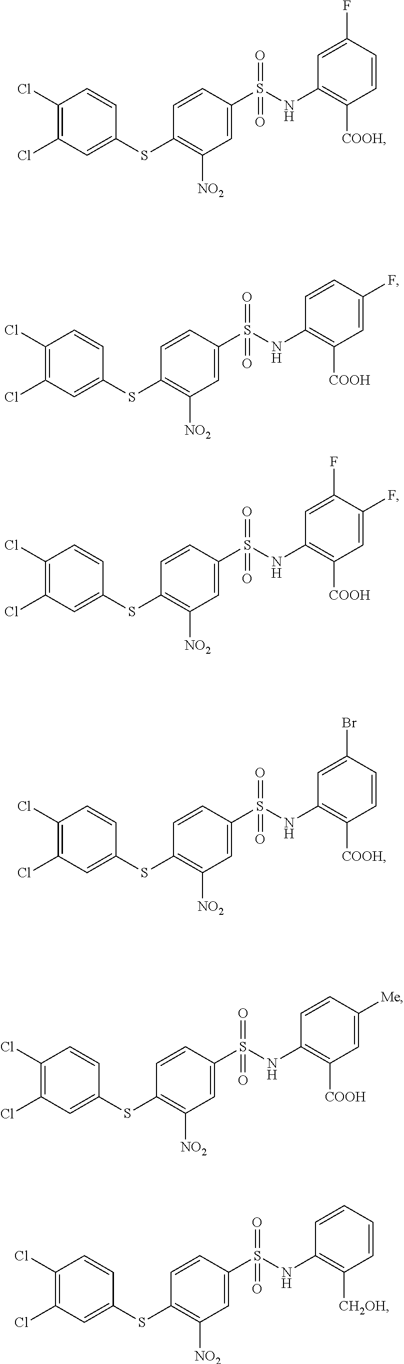 [SMILES: O=C(O)c1ccc(F)cc1NS(=O)(=O)c1ccc(Sc2ccc(Cl)c(Cl)c2)c([N+](=O)[O-])c1 ; O=C(O)c1cc(F)ccc1NS(=O)(=O)c1ccc(Sc2ccc(Cl)c(Cl)c2)c([N+](=O)[O-])c1 ; O=C(O)c1cc(F)c(F)cc1NS(=O)(=O)c1ccc(Sc2ccc(Cl)c(Cl)c2)c([N+](=O)[O-])c1 ; O=C(O)c1ccc(Br)cc1NS(=O)(=O)c1ccc(Sc2ccc(Cl)c(Cl)c2)c([N+](=O)[O-])c1 ; Cc1ccc(NS(=O)(=O)c2ccc(Sc3ccc(Cl)c(Cl)c3)c([N+](=O)[O-])c2)c(C(=O)O)c1 ; O=[N+]([O-])c1cc(S(=O)(=O)Nc2ccccc2CO)ccc1Sc1ccc(Cl)c(Cl)c1]

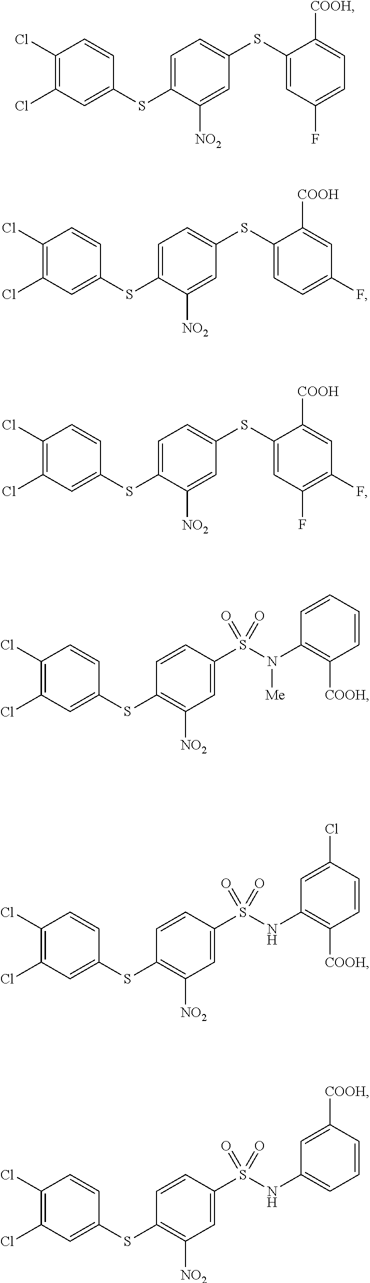 [SMILES: O=C(O)c1ccc(F)cc1Sc1ccc(Sc2ccc(Cl)c(Cl)c2)c([N+](=O)[O-])c1 ; O=C(O)c1cc(F)ccc1Sc1ccc(Sc2ccc(Cl)c(Cl)c2)c([N+](=O)[O-])c1 ; O=C(O)c1cc(F)c(F)cc1Sc1ccc(Sc2ccc(Cl)c(Cl)c2)c([N+](=O)[O-])c1 ; CN(c1ccccc1C(=O)O)S(=O)(=O)c1ccc(Sc2ccc(Cl)c(Cl)c2)c([N+](=O)[O-])c1 ; O=C(O)c1ccc(Cl)cc1NS(=O)(=O)c1ccc(Sc2ccc(Cl)c(Cl)c2)c([N+](=O)[O-])c1 ; O=C(O)c1cccc(NS(=O)(=O)c2ccc(Sc3ccc(Cl)c(Cl)c3)c([N+](=O)[O-])c2)c1]

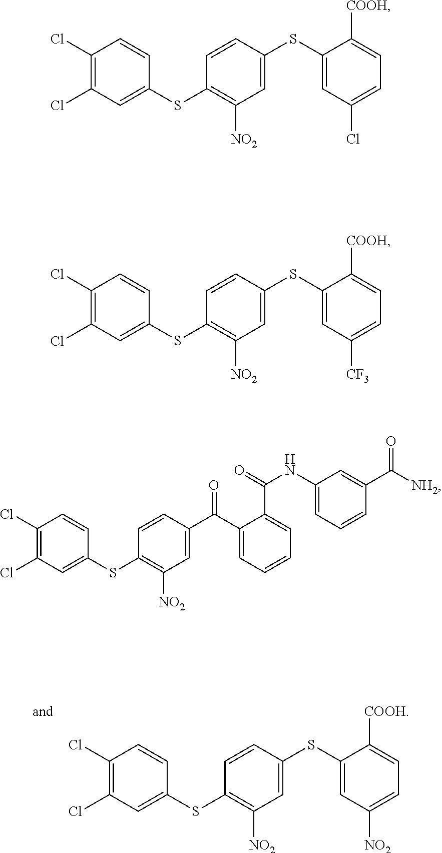 [SMILES: O=C(O)c1ccc(Cl)cc1Sc1ccc(Sc2ccc(Cl)c(Cl)c2)c([N+](=O)[O-])c1 ; O=C(O)c1ccc(C(F)(F)F)cc1Sc1ccc(Sc2ccc(Cl)c(Cl)c2)c([N+](=O)[O-])c1 ; NC(=O)c1cccc(NC(=O)c2ccccc2C(=O)c2ccc(Sc3ccc(Cl)c(Cl)c3)c([N+](=O)[O-])c2)c1 ; O=C(O)c1ccc([N+](=O)[O-])cc1Sc1ccc(Sc2ccc(Cl)c(Cl)c2)c([N+](=O)[O-])c1]

In a fourth embodiment of the third aspect, provided herein is the method of the third aspect, wherein the bacterial infection comprises Gram-positive bacteria.

### BRIEF DESCRIPTION OF THE DRAWINGS

FIG. 1 depicts (A) a homology model of B. subtilis RNAP BE. RNAP core subunits are shown in gray and σ^{A} in pale green; (B) an expanded view of the interaction between RNAP and σ^{A} showing the β′ CH region and σ_{2.2} regions; and (C) a pharmacophore model utilizing mutagenesis data overlaid on the β′ CH region with hydrogen bond acceptors in green, hydrophobic interactions in cyan, and hydrogen bond donors in magenta.

FIG. 2 depicts the chemical structure of 2-{4-[(2-aminophenyl) sulfanyl]-3-nitrobenzoyl}benzoic acid (C3).

FIG. 3 depicts a graph showing assay results of hit compound activity determined by ELISA of the β′ CH −σ^{A} interaction. Inhibitor compounds C1-C7 were used at 500 μM.

FIG. 4 depicts the MIC of exemplary compounds according to certain embodiments described herein and comparative compounds against Streptococcus agalactiae ATCC 12386 (SAGA 12386), Staphylococcus aureus ATCC 25923 and 29213 (SAUR 25923 and SAUR 29213), Staphylococcus epidermidis ATCC 12228 (SEPI 12228), Streptococcus pneumoniae ATCC 49619 (SPNE 49619), Streptococcus pyogenes ATCC 19615 (SPYO 19615), Staphylococcus saprophyticus ATCC 15305 (SSAP 15305), and Enterococcus faecium ATCC 19433 (EFAE 19433).

FIG. 5 depicts the chemical structure of exemplary compounds according to certain embodiments described herein and comparative compounds.

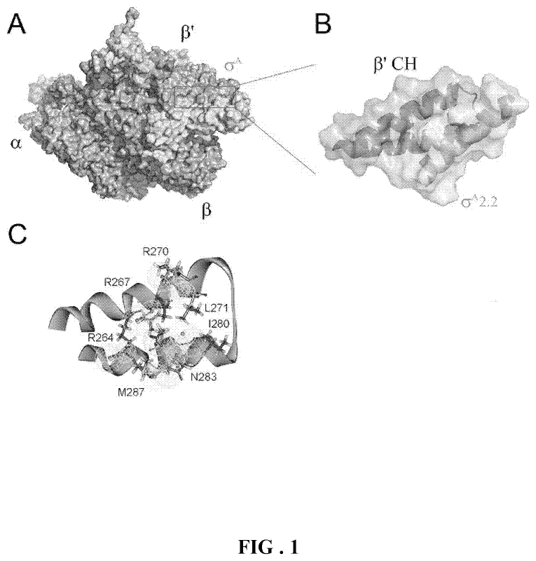

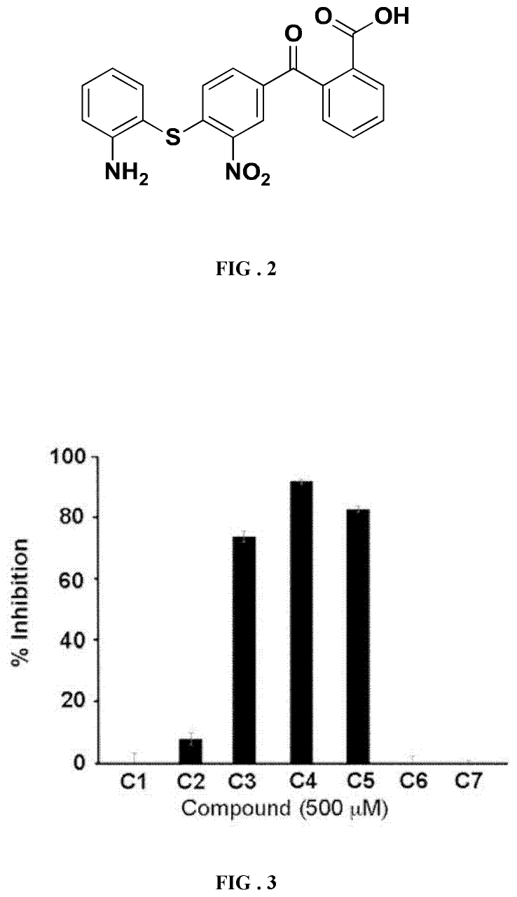

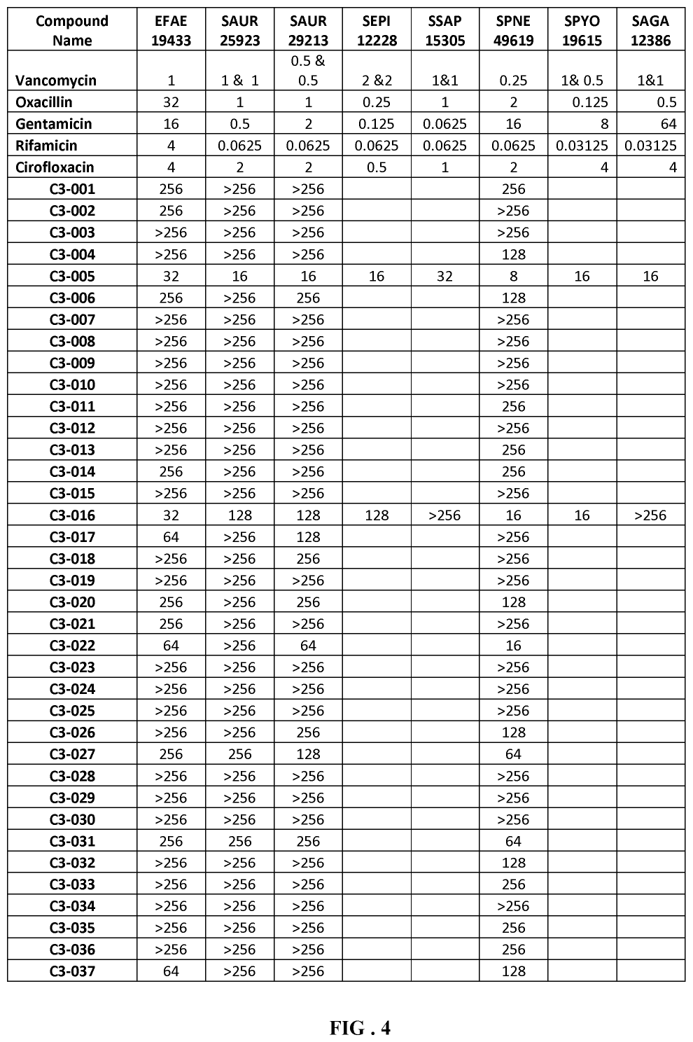

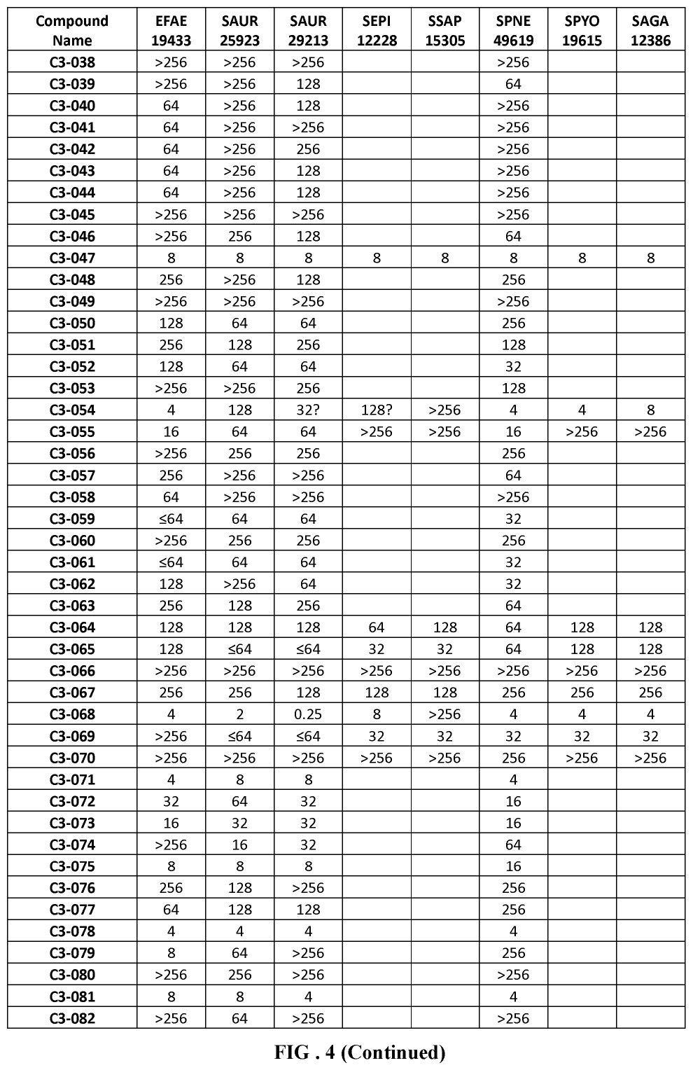

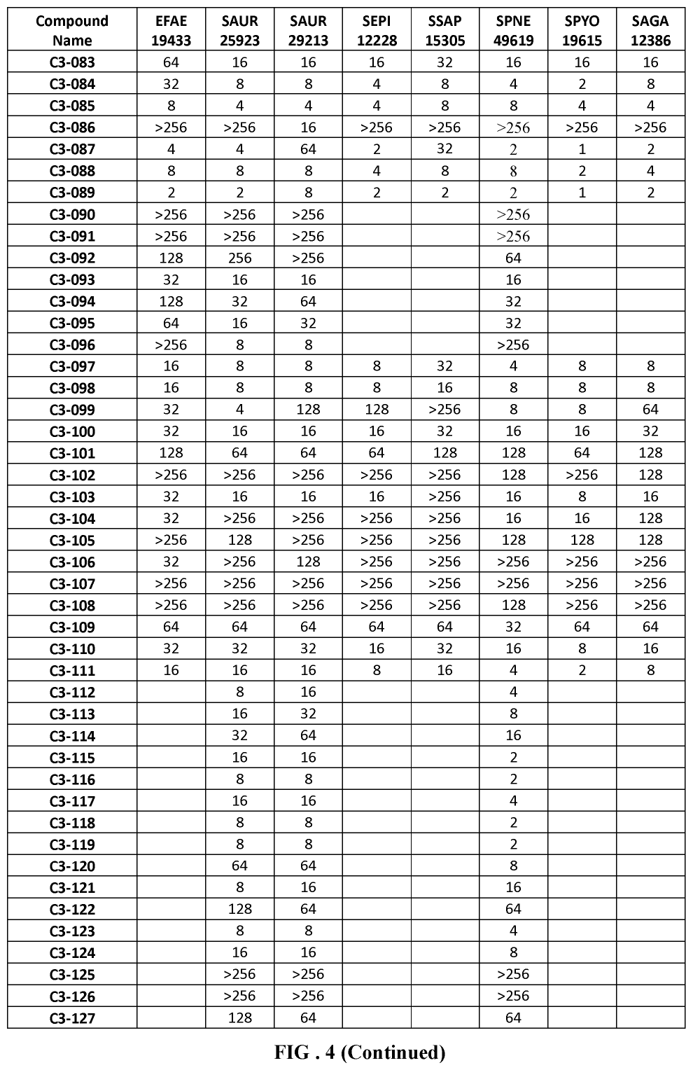

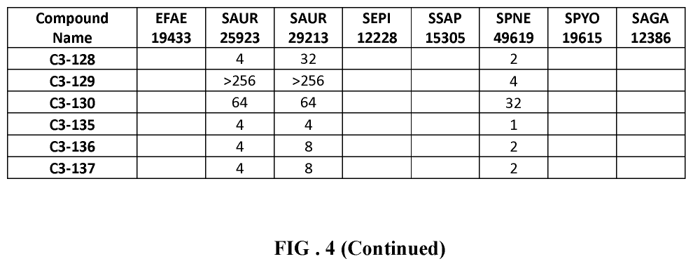

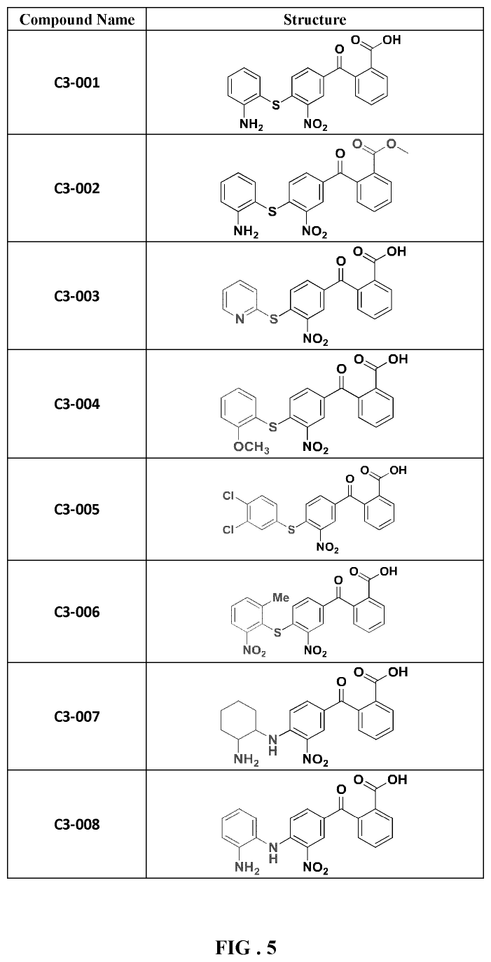

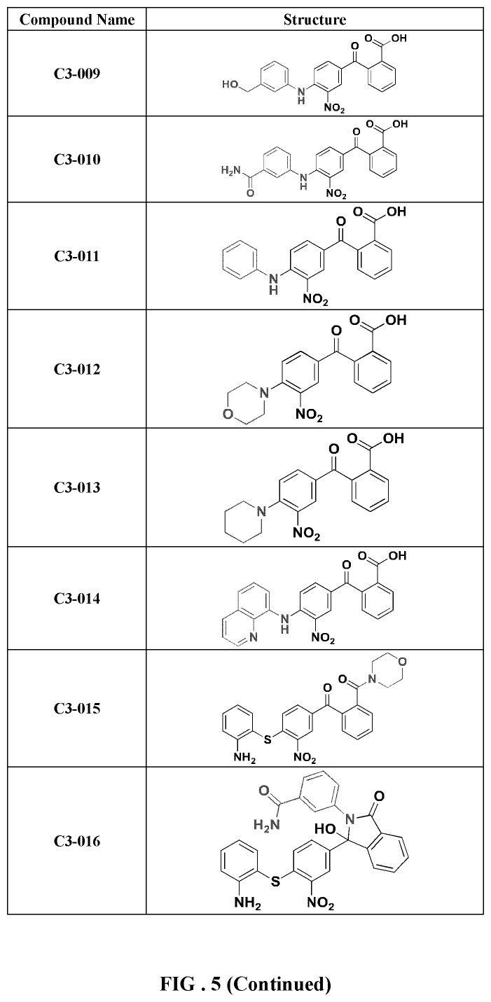

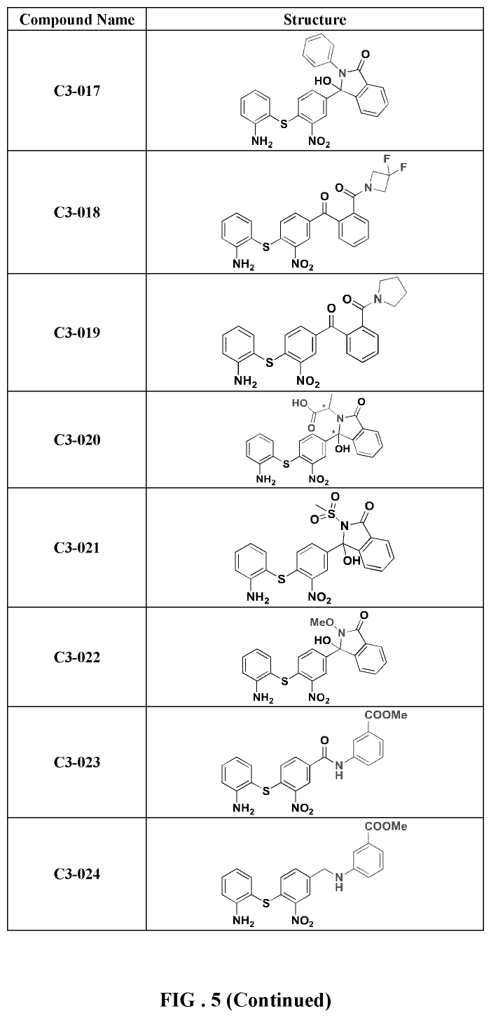

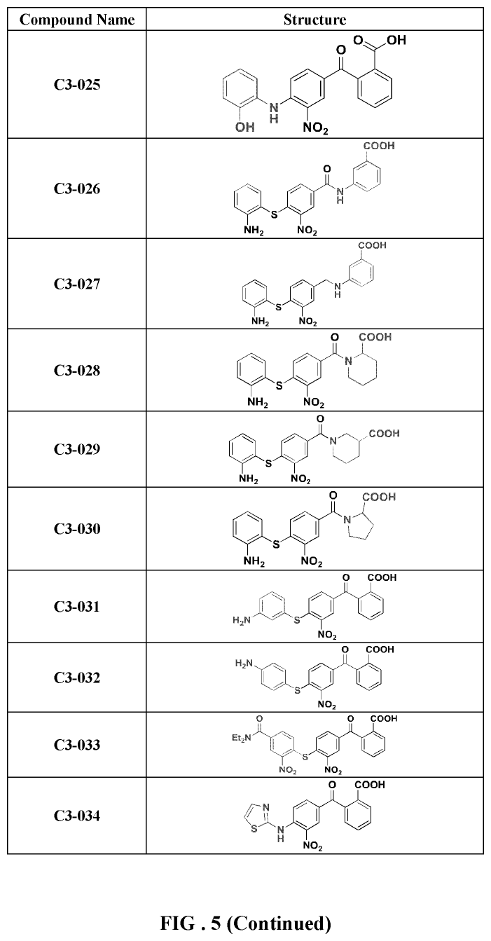

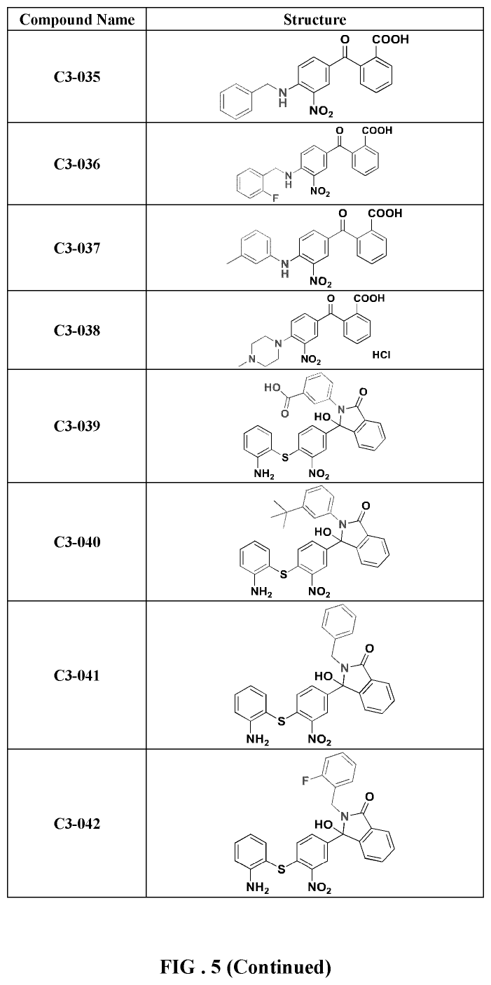

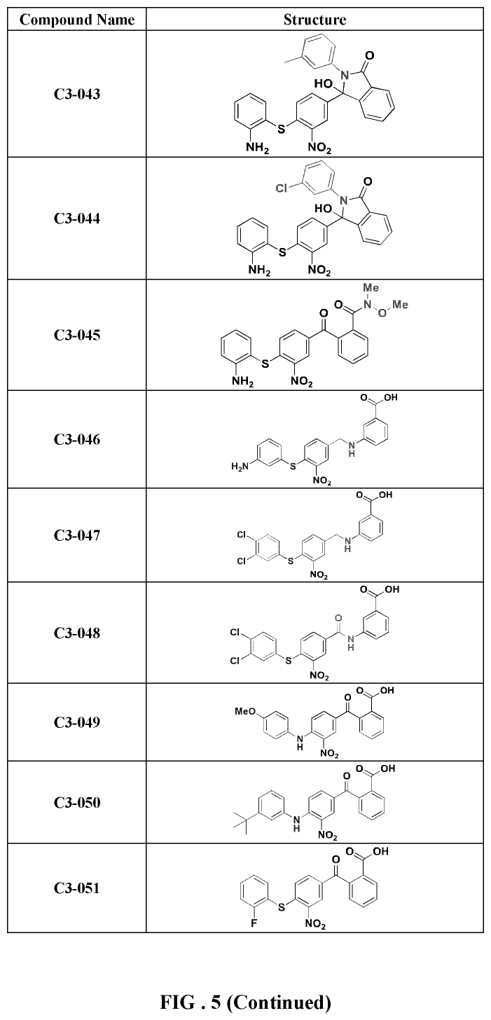

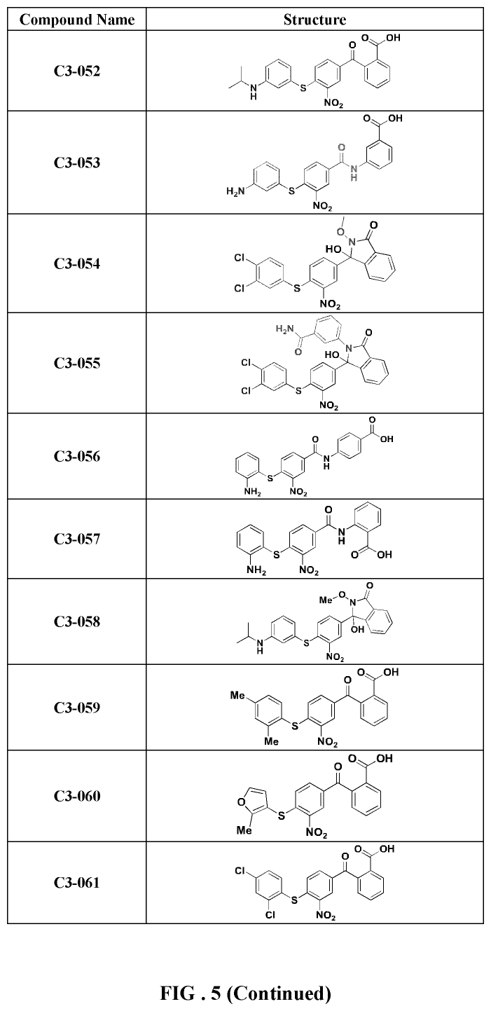

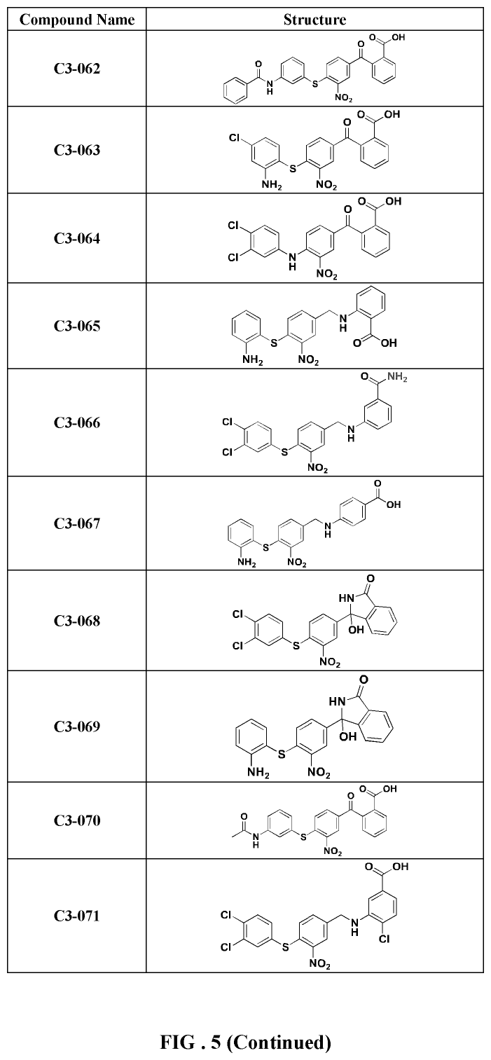

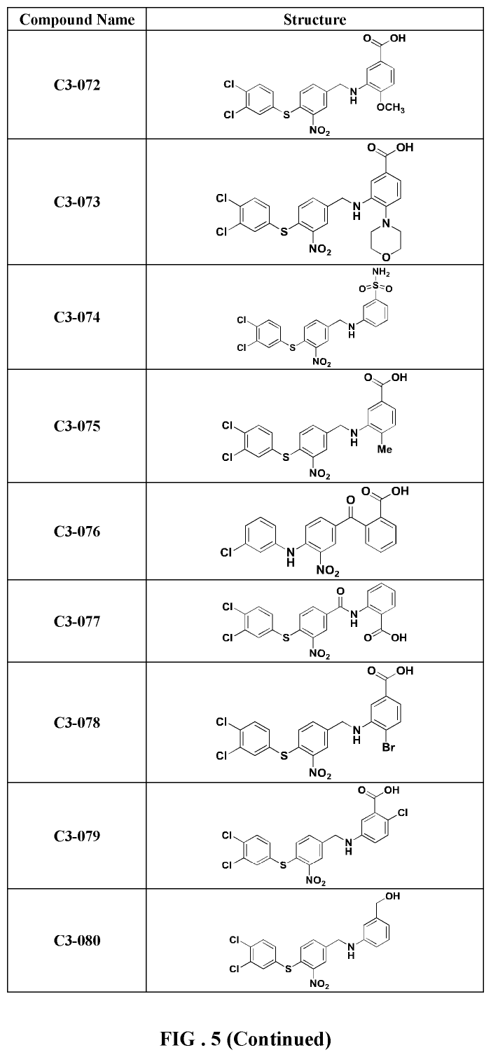

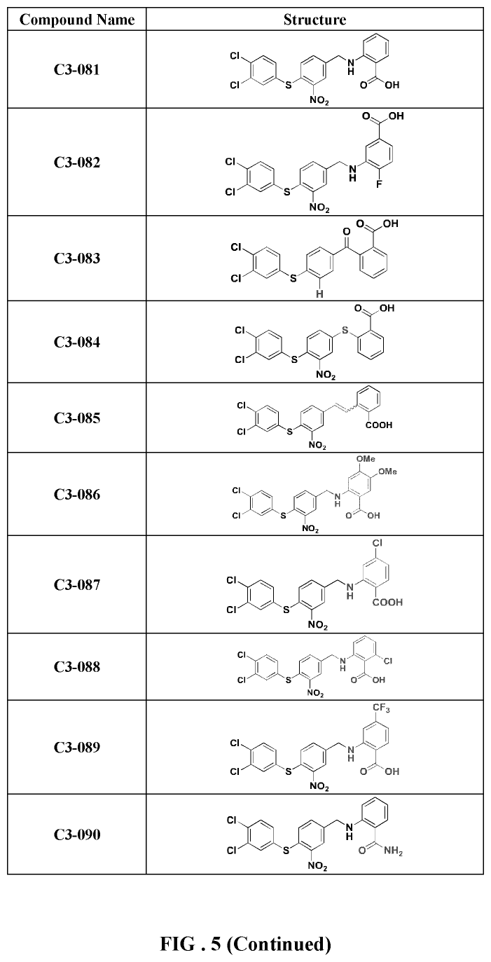

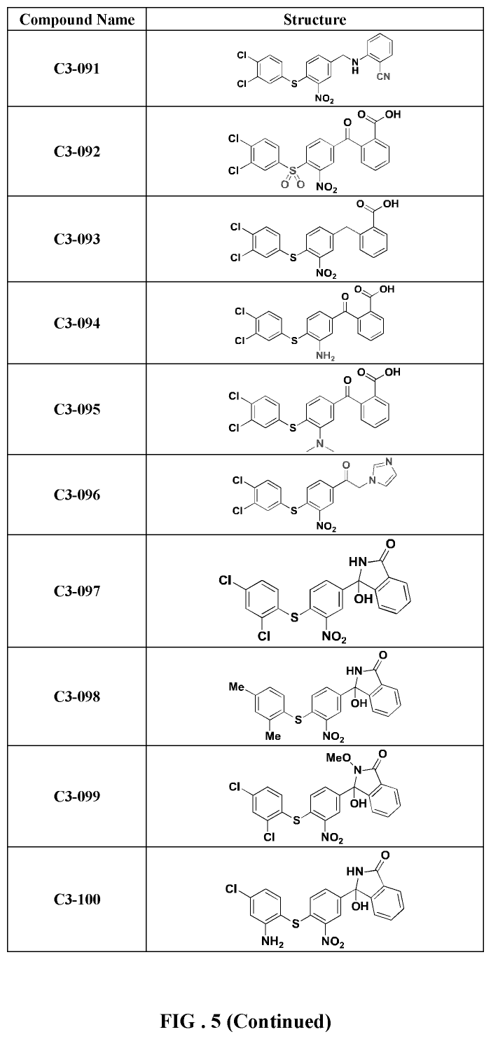

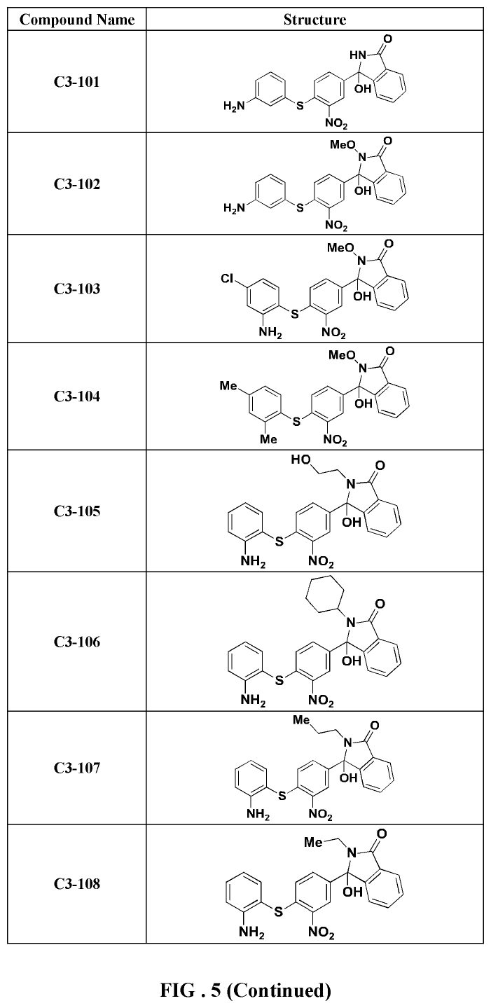

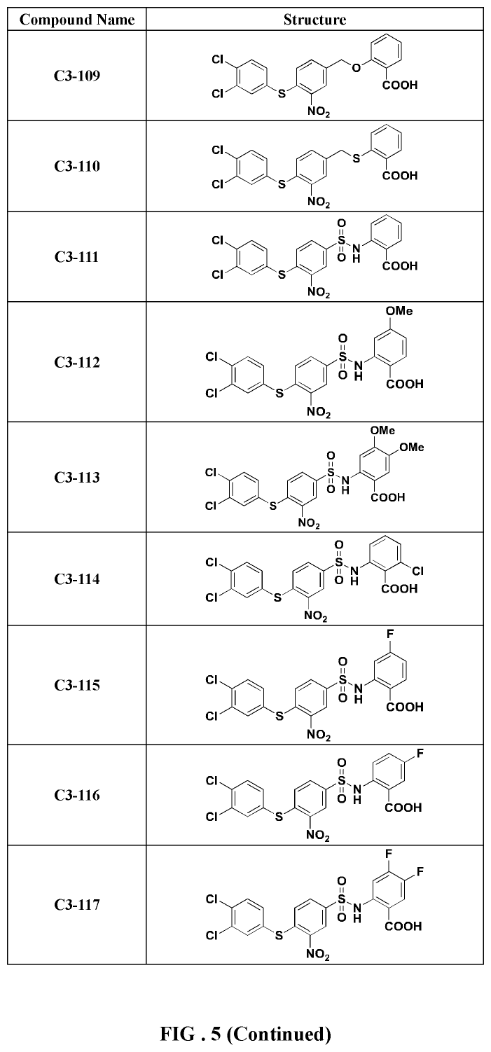

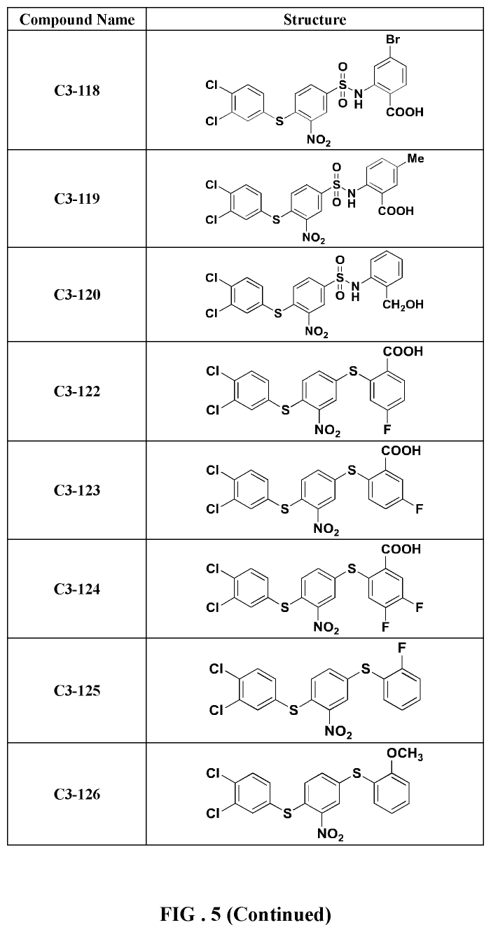

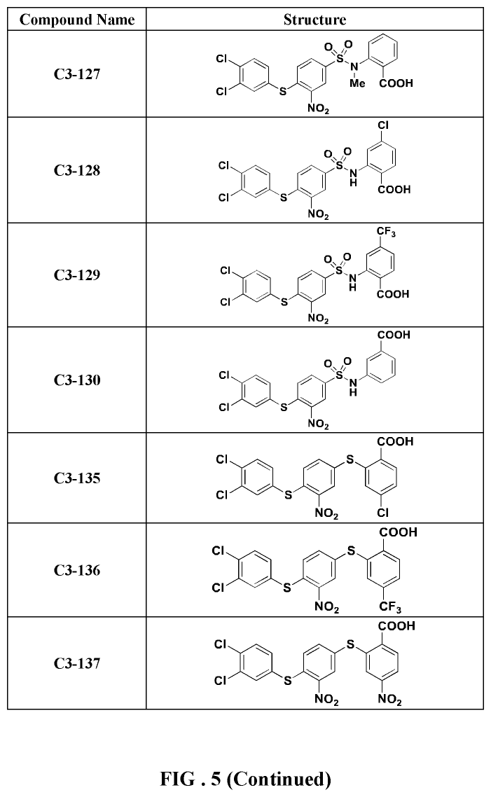

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

As used herein, unless otherwise indicated, the term “treating” means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

As used herein, unless otherwise indicated, the terms or phrases “bacterial infection(s)”, and “disorders related to bacterial infections” include the following: pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Enterococcus faecalis, E. faecium, E. casselflavus, S. epidermidis, S. haemolyticus, or Peptostreptococcus spp.; pharyngitis, rheumatic fever, and glomerulonephritis related to infection by Streptococcus pyogenes, Groups C and G streptococci, Corynebacterium diphtheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; blood and tissue infections, including endocarditis and osteomyelitis, caused by S. aureus, S. haemolyticus, E. faecalis, E. faecium, E. durans, including strains resistant to known antibacterials such as, but not limited to, beta-lactams, vancomycin, aminoglycosides, quinolones, chloramphenicol, tetracylines and macrolides; uncomplicated skin and soft tissue infections and abscesses, and puerperal fever related to infection by Staphylococcus aureus, coagulase-negative staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus aureus, coagulase-negative staphylococcal species, or Enterococcus spp.; urethritis and cervicitis; sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; infections caused by Mycobacterium tuberculosis, M. leprae, M. paratuberculosis, M. kansasii, or M. chelonei; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis or cardiovascular disease related to infection by Helicobacter pylori or Chlamydia pneumoniae. Bacterial infections and disorders related to such infections, which may be treated or prevented in animals include the following: bovine respiratory disease related to infection by P. haemolytica, P. multocida, Mycoplasma bovis, or Bordetella spp.; cow enteric disease related to infection by E. coli or protozoa (i.e., coccidia, cryptosporidia, etc.); dairy cow mastitis related to infection by S. aureus, Strep. uberis, Streptococcus agalactiae, Streptococcus dysgalactiae, Klebsiella spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodysinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and disorders related to such infections, which may be treated or prevented in accord with the method of the present invention are referred to in J. P. Sanford et al., “The Sanford Guide To Antimicrobial Therapy,” 26 th Edition, (Antimicrobial Therapy, Inc., 1996).

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “therapeutically effective amount” as used herein, means that amount of active compound or pharmaceutical agent that elicits a biological, medicinal, or imaging response in a cell culture, tissue system, subject, animal, or human that is being sought by a researcher, veterinarian, clinician, or physician, which includes alleviation of the symptoms of the disease, condition, or disorder being treated and/or achieving the desired degree of magnetic resonance imaging contrast enhancement.

The term “composition” is intended to encompass a product comprising the specified ingredients in the specified amounts, as well as any product that results, directly or indirectly, from combinations of the specified ingredients in the specified amounts.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

As used herein, unless otherwise indicated, the term “halo” or “halide” includes fluoro, chloro, bromo or iodo. Preferred halo groups are fluoro, chloro and bromo.

As used herein, unless otherwise indicated, the term “alkyl” includes saturated monovalent hydrocarbon radicals having cyclic, straight and/or branched moieties.

As used herein, unless otherwise indicated, the term “alkenyl”, as used herein, unless otherwise indicated, includes alkyl groups as defined above having at least one carbon-carbon double bond at some point in the alkyl chain.

As used herein, unless otherwise indicated, the term “alkynyl”, as used herein, unless otherwise indicated, includes alkyl groups as defined above having at least one carbon-carbon triple bond at some point in the alkyl chain.

As used herein, unless otherwise indicated, the term “aryl” includes an organic radical derived from an aromatic hydrocarbon by removal of one hydrogen, such as phenyl or naphthyl.

As used herein, unless otherwise indicated, the term “4 to 10 membered heterocyclic” includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl. benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

Provided herein are compounds useful as antibacterial agents. In certain embodiments, the compound has the Formula 1:

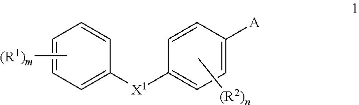

or a pharmaceutically acceptable salt, solvate, or tautomer thereof, wherein

A represents a moiety selected from the group consisting of:

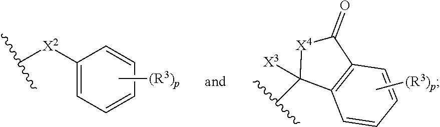

m is a whole number selected from 1-5;

n is a whole number selected from 1-4;

p is a whole number selected from 1-4;

q is a whole number selected from 2-5;

X¹ is —S—, —O—, —C(R)₂—, or —N(R)—;

X² is —S—, —O—, —C(R)₂—, —N(R)—, —C(O)—, —C(S)—, —C(O)C(R)₂—*, —C(R)₂C(O)—*, —N═CH—*, —(CR₂)_{q}—, —CH═N—*, —(R)C═C(R)—*, —C≡C—*, —NRC(R)₂—*, —CR₂N(R)—*, —OC(R)₂—*, —C(R)₂—O—*, —SC(R)₂—*, —C(R)₂S—*, —N(R)C(O)—*, —C(O)N(R)—*, —CR(OH)C(R)₂—*, —CR₂CR(OH)—*, —S(O)C(R)₂—*, —S(O)₂C(R)₂—*, —C(R)₂S(O)—*, —C(R)₂S(O)₂—*, —S(O)CR(OH)—*, —S(O)₂CR(OH)—*, —CR(OH)S(O)—*, —CR(OH)S(O)₂—*, —S(O)N(R)—*, —S(O)₂N(R)—*, —N(R)S(O)—*, —NRS(O)₂—*, —S(O)NR(OH)—*, —S(O)₂NR(OH)—*, —NR(OH)S(O)—*, or —NR(OH)S(O)₂—*, wherein * indicates the position of a covalent bond with the moiety:

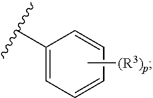

X³ is hydrogen or —OR;

X⁴ is —O—, —N(OR⁴)—, or —N(R⁴)—;

R for each instance is independently selected from the group consisting of hydrogen, alkyl, haloalkyl, alkenyl, alkynyl, cycloalkyl, heterocycloalkyl, aryl, heteroaryl, or aralkyl; or two instances of R together with the atom they are covalently bonded form a 3-6 membered cycloalkyl and heterocyloalkyl;

R¹ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;

R² for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)—N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;

R³ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; and

R⁴ is selected from the group consisting of hydrogen, alkyl, haloalkyl, alkenyl, alkynyl, cycloalkyl, heterocycloalkyl, aryl, heteroaryl, aralkyl, or —(CR₂)ₜY, wherein t is 1-10 and Y is halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkenyl, alkynyl, cycloalkyl, heterocycloalkyl, aryl, heteroaryl, and aralkyl.

In certain embodiments the compound of Formula 1 excludes compounds selected from the group consisting of:

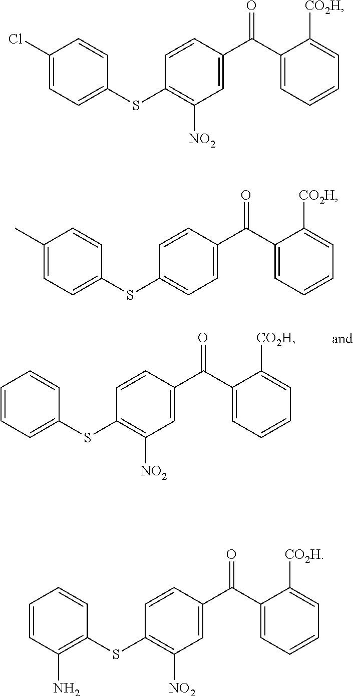

The compounds described herein can encompass different positional isomers indicated by a bond that is not attached to the vertex of a chemical structure, such as illustrated using the model structure below:

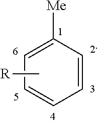

In this model structure, the group R can be connected to any atom on the ring structure, valency permitting, i.e., carbons 2, 3, 4, 5, or 6 in the structure above.

Tautomeric forms of the compounds described herein are also contemplated by the present disclosure. Such tautomeric forms include, but are not limited to tautomeric forms of ketones, amides, esters, and the like. Under certain conditions, when A is represented by the moiety:

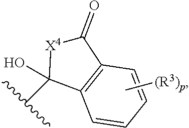

it may exist in equilibrium with its ring open tautomeric form. This equilibrium is shown below:

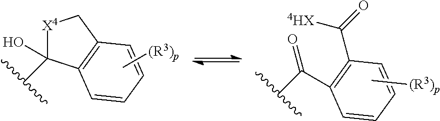

Depending on the structure of the compound of Formula 1 and the chemical environment of the compound (e.g., solvent polarity, hydrogen bonding capacity of solvent, temperature, pH, etc), the equilibrium may shift partially or even entirely to the tautomeric ring open or ring closed form. In certain embodiments of the compound of Formula 1, wherein A is the moiety:

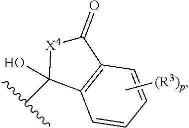

the compound of Formula 1 does not include the tautomeric ring opened form shown below:

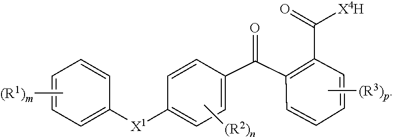

In certain embodiments of the compound of Formula 1, wherein A is the moiety:

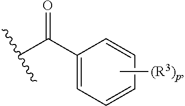

the compound of Formula 1 does not include the tautomeric ring closed form shown below:

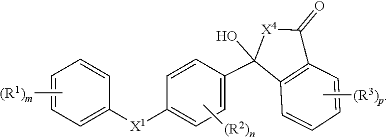

The number of covalently attached substituents (R¹, R², and R³) on each phenyl ring can be varied independently. In certain embodiments, m is a whole number selected from 1-4, 1-3, or 1-2. In certain embodiments, n is a whole number selected from 1-3. In certain embodiments, n is a whole number selected from 1-3 or 1-2. In certain embodiments, m is a whole number selected from 1-2. In certain embodiments, p is a whole number selected from 1-3 or 1-2. In certain embodiments, m is a whole number selected from 1-2.

X¹ can be —S—, —O—, —C(R)₂—, or —N(R)—, wherein each R is independently selected from the group consisting of hydrogen, C₁-C₆ alkyl, C₃-C₆ cycloalkyl, heterocycloalkyl, aryl, heteroaryl, or aralkyl; or two instances of R together with the atom they are covalently bonded form a 3-6 membered cycloalkyl. In certain embodiments, X¹ is —S—, —O—, —CH₂—, —NH—, or —N(C₁-C₆ alkyl)-. In certain embodiments, X¹ is —S— or —O—.

In instances in which A is represented by the moiety:

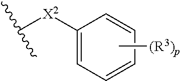

the compound of Formula 1, can be represented by the compound of Formula 1A:

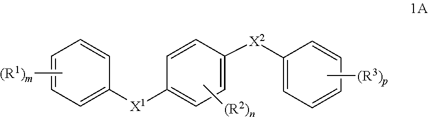

X² can be —S—, —O—, —C(R)₂—, —N(R)—, —C(O)—, —C(S)—, —C(O)C(R)₂—*, —C(R)₂C(O)—*, —N═CH—*, —(CR₂)_{q}—, —CH═N—*, —(R)C═C(R)—*, —C≡C—*, —NRC(R)₂—*, —CR₂N(R)—*, —OC(R)₂—*, —C(R)₂—O—*, —SC(R)₂—*, —C(R)₂S—*, —N(R)C(O)—*, —C(O)N(R)—*, —CR(OH)C(R)₂—*, —CR₂CR(OH)—*, —S(O)C(R)₂—*, —S(O)₂C(R)₂—*, —C(R)₂S(O)—*, —C(R)₂S(O)₂—*, —S(O)CR(OH)—*, —S(O)₂CR(OH)—*, —CR(OH)S(O)—*, —CR(OH)S(O)₂—*, —S(O)N(R)—*, —S(O)₂N(R)—*, —N(R)S(O)—*, —NRS(O)₂—*, —S(O)NR(OH)—*, —S(O)₂NR(OH)—*, —NR(OH)S(O)—*, or —NR(OH)S(O)₂—*. The symbol * indicates the position of a covalent bond with the moiety:

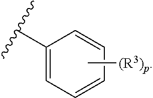

For example, when X² is —C(O)C(R)₂—*, the compound of Formula 1 can be represented by the structure:

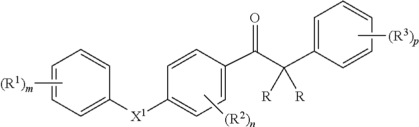

In certain embodiments, X² is —S—, —O—, —C(R)₂—, —C(O)—, —C(S)—, —C(O)C(R)₂—*, —(R)C═C(R)—*, —CR₂N(R)—*, —C(R)₂—O—*, —C(R)₂S—*, or —S(O)₂N(R)—*, wherein each R is independently hydrogen, C₁-C₆ alkyl, C₃-C₆ cycloalkyl, heterocycloalkyl, aryl, heteroaryl, or aralkyl; or each R is independently hydrogen or C₁-C₆ alkyl. In certain embodiments, X² is —S—, —C(R)₂—, —C(O)—, —C(O)C(R)₂—*, —(R)C═C(R)—*, —CR₂N(R)—*, or —S(O)₂N(R)—*, wherein each R is independently hydrogen or C₁-C₆ alkyl; or R is hydrogen.

In instances in which A is represented by the moiety:

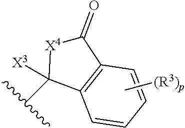

the compound of Formula 1, can be represented by the compound of Formula 1B:

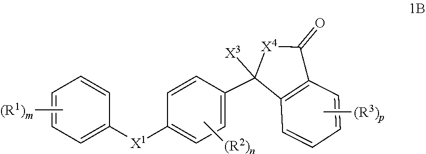

In certain embodiments, X³ is hydrogen or —OR, wherein each R is independently hydrogen, C₁-C₆ alkyl, C₃-C₆ cycloalkyl, heterocycloalkyl, aryl, heteroaryl, or aralkyl; or R is hydrogen.

In certain embodiments, X⁴ is —N(OR⁴)— or —N(R⁴)—, wherein R⁴ is hydrogen alkyl, haloalkyl, alkenyl, alkynyl, cycloalkyl, heterocycloalkyl, aryl, heteroaryl, aralkyl, or —(CR₂)ₜY, wherein t is 1-10 and Y is halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkenyl, alkynyl, cycloalkyl, heterocycloalkyl, aryl, heteroaryl, and aralkyl. In certain embodiments, X⁴ is —N(OR₄)— or —N(R⁴)—, wherein R⁴ is hydrogen alkyl, cycloalkyl, aryl, heteroaryl, or —(CR₂)ₜY

In certain embodiments, Y is —OR, —SR, —N(R)₂, —OC(O)R, —N(R)C(O)R, —OC(O)OR, or —N(R)S(O)₂R; or Y is OR, —SR, or —N(R)₂. In certain embodiments, t is 1-8, 2-8, 2-6, or 2-4. In certain embodiments, t is 2.

In certain embodiments, R¹ for each instance is independently selected from the group consisting of hydrogen, halide, nitrile, nitro, —N(R)₂, —N(R)C(O)R, alkyl, and haloalkyl. In certain embodiments, each R¹ is independently hydrogen, halide —N(R)₂, —N(R)C(O)R, C₁-C₆ alkyl, wherein each R is independently hydrogen, C₁-C₆ alkyl, C₃-C₆ cycloalkyl, heterocycloalkyl, aryl, heteroaryl, or aralkyl.

In certain embodiments, the compound of Formula 1 is selected from the group consisting of.

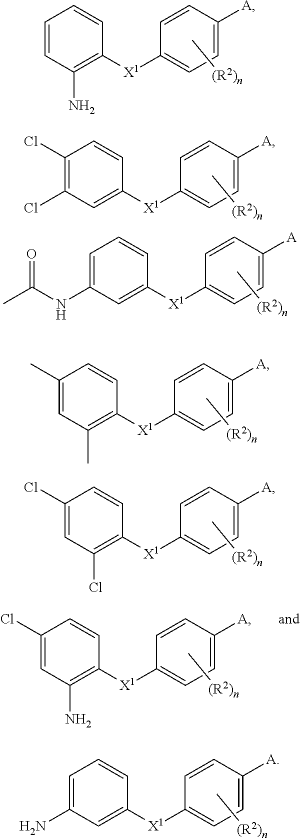

In certain embodiments, R² for each instance is independently selected from the group consisting of hydrogen, nitro, and —N(R)₂, wherein each R is independently hydrogen, C₁-C₆ alkyl, C₃-C₆ cycloalkyl, heterocycloalkyl, aryl, heteroaryl, or aralkyl; or each R is independently hydrogen or C₁-C₆ alkyl. In certain embodiments, R² for each instance is independently selected from the group consisting of hydrogen, nitro, and —NH₂.

In certain embodiments, R³ for each instance is independently selected from the group consisting of hydrogen, halide, nitrile, nitro, azido, —OR, —SR, —N(R)₂, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂N(R)₂, —N(R)S(O)₂R, perhaloalkoxyl, alkyl, alkenyl, alkynyl, cycloalkyl, heterocycloalkyl, aryl, heteroaryl, and aralkyl. In certain embodiments, R³ for each instance is independently selected from the group consisting of hydrogen, halide, —OR, —C(O)N(R)₂, perhaloalkoxyl, alkyl, and heterocycloalkyl.

In certain embodiments, the compound of Formula 1 has the Formula 2:

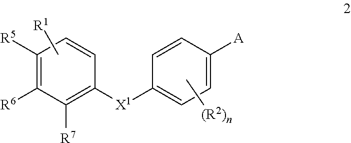

wherein each of R⁵ and R⁶ is independently selected from the group consisting of halide, —N(R)₂, and alkyl and R⁷ is hydrogen; each of R⁵ and R⁷ is independently selected from the group consisting of halide, —N(R)₂, and alkyl and R⁶ is hydrogen; or R⁷ is hydrogen or —N(R)₂ and R⁵ and R⁶ is hydrogen.

In certain embodiments, the compound of Formula 1 has the Formula 3:

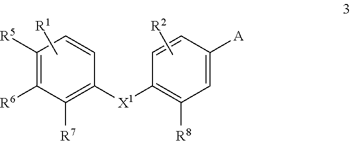

wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl; and

R⁸ is hydrogen, nitro, —N(R)₂, —N(R)C(O)R, —N(R)C(O)OR, —N(R)C(O)N(R)₂, —N(R)S(O)₂R, or perhaloalkoxyl, wherein at least one of R⁵, R⁶, and R⁷ is not hydrogen; or each of R⁵ and R⁶ is independently selected from the group consisting of halide, —N(R)₂, and alkyl; R⁷ is hydrogen;

each of R⁵ and R⁷ is independently selected from the group consisting of halide, —N(R)₂, and alkyl; R⁶ is hydrogen; and R⁸ is hydrogen, nitro, or —N(R)₂, wherein each R is independently hydrogen, C₁-C₆ alkyl, C₃-C₆ cycloalkyl, heterocycloalkyl, aryl, heteroaryl, or aralkyl; or

R⁵ and R⁶ is hydrogen; R⁷ is hydrogen or —N(R)₂; and R⁸ is hydrogen, nitro, or —N(R)₂, wherein each R is independently hydrogen, C₁-C₆ alkyl, C₃-C₆ cycloalkyl, heterocycloalkyl, aryl, heteroaryl, or aralkyl.

In certain embodiments, the compound is selected from a compound of Formula I and a compound of Formula II:

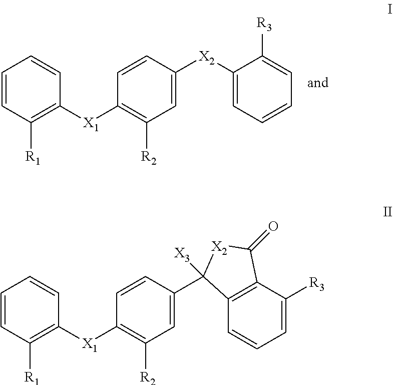

or a pharmaceutically acceptable salt, prodrug, or solvate thereof, wherein:

X₁, X₂ and X₃ are O, NHn, —S(O)ₙ, —S(O)˜ OH, C₁-C₅ alkyl, alkenyl, alkynyl, aryl wherein carbons of R₁, R₂ and R₃ are optionally replaced by heteroatoms selected from a group consisting of O, S, N, and said R₁, R₂ or R₃ is optionally substituted selected from the group consisting of halo, cyano, nitro, trifluoromethoxy, trifluoromethyl, azido, hydroxy, —C(O)OH, —NH₂, —NHC(O)(C₁-C₁₀ alkyl, alkenyl, alkynyl, aryl, heterocyclic), —S(O)˜ OH wherein n is an integer ranging from 0 to 2. E.g. when X₁, X₂ or X₃ contains two atoms, it can be —N═CH—, —CH═N—, —NH—CH₂—, —CH₂—NH—, —O—CH₂—, —CH₂—O—, —S—CH₂—, —CH₂—S—, —NH—C(O)—, —C(O)—NH—, —N(OH)—CH₂—, —CH₂—N(OH)—, —CH(OH)—CH₂—, —CH₂—CH(OH)—, —O—CH(OH)—, —CH(OH)—O—, —S—CH(OH)—, —CH(OH)—S—, —S(O)—CH₂—, —S(O)₂—CH₂—, —CH₂—S(O)—, —CH₂—S(O)₂—, —S(O)—CH(OH)—, —S(O)₂—CH(OH)—, —CH(OH)—S(O)—, —CH(OH)—S(O)₂—, —S(O)—NH—, —S(O)₂—NH—, —NH—S(O)—, —NH—S(O)₂—, —S(O)—NH(OH)—, —S(O)₂—NH(OH)—, —NH(OH)—S(O)—, —NH(OH)—S(O)₂—; Each R₁, R₂ and R₃ is C1-C10 alkyl, alkenyl, alkynyl, aryl wherein carbons of R₁, R₂ and R₃ are optionally replaced by heteroatoms selected from a group consisting of O, S, N, and said R₁, R₂ or R₃ is optionally substituted selected from the group consisting of O, S, N, halo, cyano, nitro, trifluoromethoxy, trifluoromethyl, azido, hydroxy, —C(O)OH, —NH₂, —NHC(O)(C₁-C₁₀ alkyl, alkenyl, alkynyl, aryl, heterocyclic), —S(O)˜ OH wherein n is an integer ranging from 0 to 2; R₁ or R₂ is one or multiple substituents on any position of the phenyl ring related to X₁ of the Formula I and 2; R₂ or R₃ is one or multiple substituents on any position of the phenyl ring related to X₂ of the Formula I; R₂ is one or multiple substituents on any position of the phenyl ring related to the bicyclic ring containing R₃ of the Formula II; R₃ is one or multiple substituents on any position of the phenyl ring of the bicyclic system; The three phenyl rings of the Formula I and II are optionally replaced by aryl, cyclic alkyl, heterocyclyl, the bicyclic ring system of the Formula II is optionally replaced by aryl, cyclic alkyl, heterocyclyl, the three phenyl rings of the Formula I and II and bicyclic ring system of formula 2 are optionally substituted by selected from the group consisting of O, S, N, halo, cyano, nitro, trifluoromethoxy, trifluoromethyl, azido, hydroxy, —C(O)OH, —NH₂, —NHC(O)(C₁-C₁₀ alkyl, alkenyl, alkynyl, aryl, heterocyclic), —S(O)˜ OH wherein n is an integer ranging from 0 to 2, each R₁, R₂, R₃ is independently selected from halo, cyano, nitro, trifluoromethoxy, trifluoromethyl, azido, hydroxy, C₁-C₁₀ alkoxy, C₁-C₁₀ alkyl, C₂-C₁₀ alkenyl, C₂-C₁₀ alkynyl, —C(O)(C₁-C₁₀ alkyl, alkenyl, alkynyl, aryl, heterocyclyl), —C(O)O(C₁-C₁₀ alkyl, alkenyl, alkynyl, aryl, heterocyclic), —OC(O) (C₁-C₁₀ alkyl, alkenyl, alkynyl, aryl, heterocyclic), —N(C₁-C₁₀ alkyl, alkenyl, alkynyl, aryl, heterocyclic)C(O) (C₁-C₁₀ alkyl, alkenyl, alkynyl, aryl, heterocyclic), —N(C₁-C₁₀ alkyl, alkenyl, alkynyl, aryl, heterocyclic)C(O)N(C₁-C₁₀ alkyl, alkenyl, alkynyl, aryl, heterocyclic), —N(C₁-C₁₀ alkyl, alkenyl, alkynyl, aryl, heterocyclic)C(O)O(C₁-C₁₀ alkyl, alkenyl, alkynyl, aryl, heterocyclic), —C(O)N(C₁-C₁₀ alkyl, alkenyl, alkynyl, aryl, heterocyclic), —N(C₁-C₁₀ alkyl, alkenyl, alkynyl, aryl, heterocyclic), —N(C₁-C₁₀ alkyl, alkenyl, alkynyl, aryl, heterocyclic)O(C₁-C₁₀ alkyl, alkenyl, alkynyl, aryl, heterocyclic), —SO₂N(C₁-C₁₀ alkyl, alkenyl, alkynyl, aryl, heterocyclic), —S(O)ⱼ(C₁-C₁₀ alkyl, alkenyl, alkynyl, aryl, heterocyclic) wherein j is an integer from 0 to 2.

In certain embodiments, the compound of Formula 1 is selected from the group consisting of:

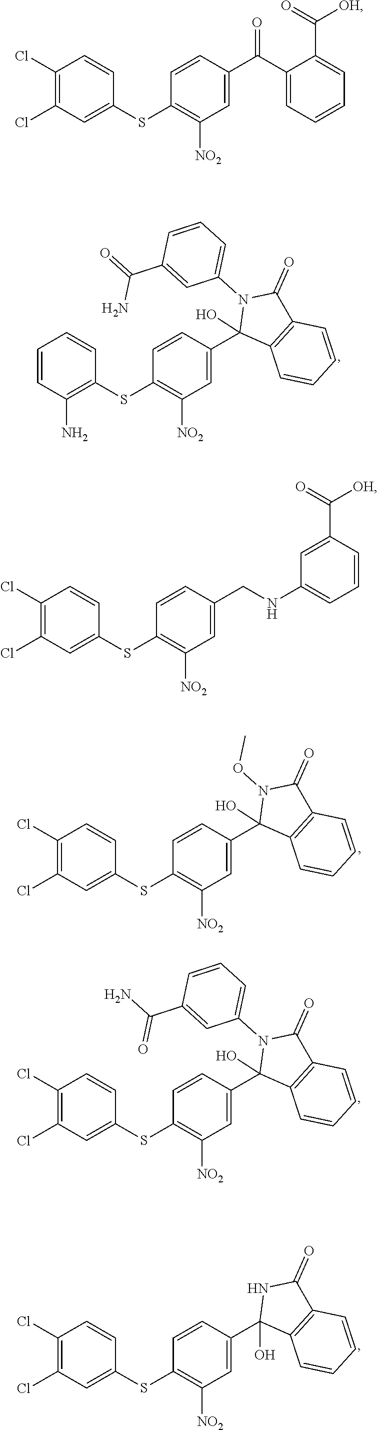

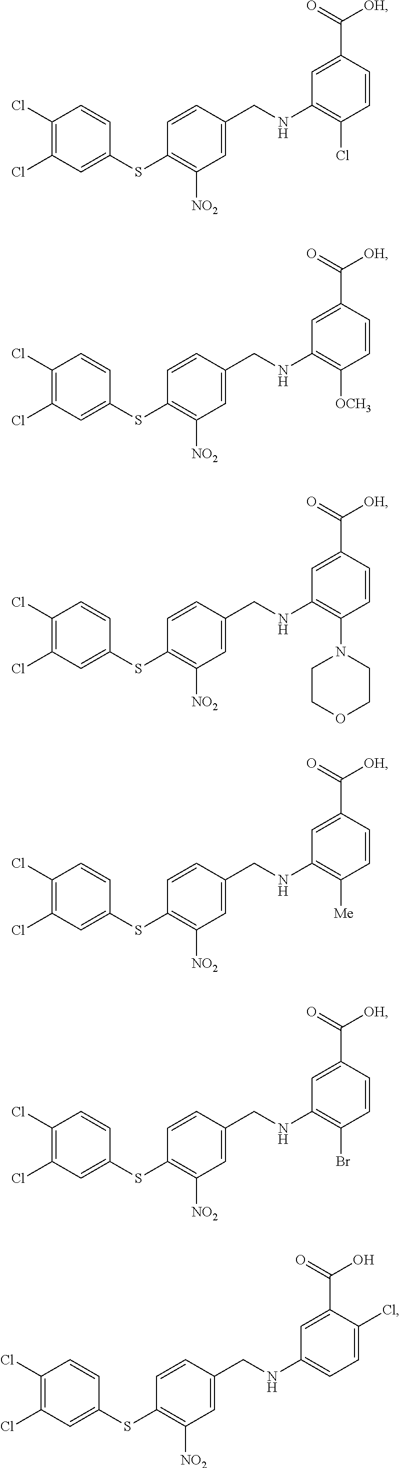 [SMILES: O=C(O)c1ccc(Cl)c(NCc2ccc(Sc3ccc(Cl)c(Cl)c3)c([N+](=O)[O-])c2)c1 ; COc1ccc(C(=O)O)cc1NCc1ccc(Sc2ccc(Cl)c(Cl)c2)c([N+](=O)[O-])c1 ; O=C(O)c1ccc(N2CCOCC2)c(NCc2ccc(Sc3ccc(Cl)c(Cl)c3)c([N+](=O)[O-])c2)c1 ; Cc1ccc(C(=O)O)cc1NCc1ccc(Sc2ccc(Cl)c(Cl)c2)c([N+](=O)[O-])c1 ; O=C(O)c1ccc(Br)c(NCc2ccc(Sc3ccc(Cl)c(Cl)c3)c([N+](=O)[O-])c2)c1 ; O=C(O)c1cc(NCc2ccc(Sc3ccc(Cl)c(Cl)c3)c([N+](=O)[O-])c2)ccc1Cl]

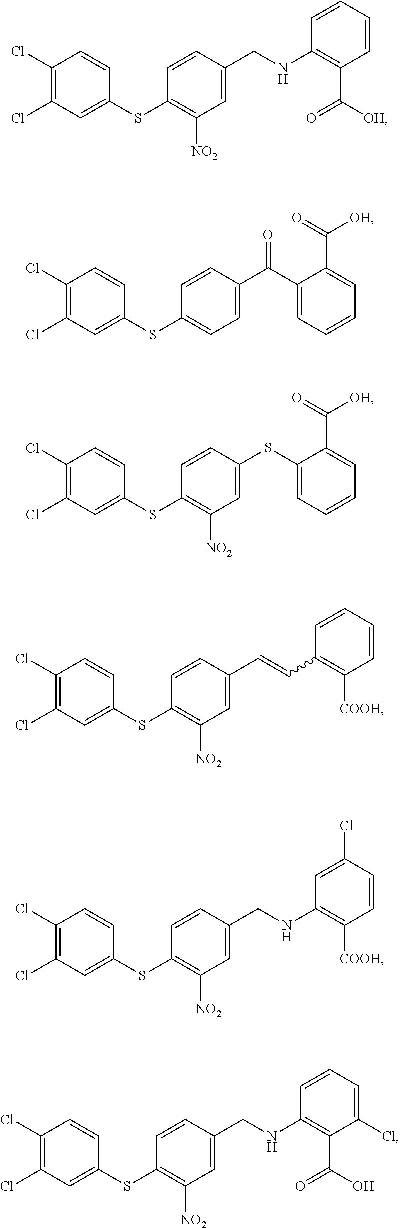 [SMILES: O=C(O)c1ccccc1NCc1ccc(Sc2ccc(Cl)c(Cl)c2)c([N+](=O)[O-])c1 ; O=C(O)c1ccccc1C(=O)c1ccc(Sc2ccc(Cl)c(Cl)c2)cc1 ; O=C(O)c1ccccc1Sc1ccc(Sc2ccc(Cl)c(Cl)c2)c([N+](=O)[O-])c1 ; O=C(O)c1ccccc1C=Cc1ccc(Sc2ccc(Cl)c(Cl)c2)c([N+](=O)[O-])c1 ; O=C(O)c1ccc(Cl)cc1NCc1ccc(Sc2ccc(Cl)c(Cl)c2)c([N+](=O)[O-])c1 ; O=C(O)c1c(Cl)cccc1NCc1ccc(Sc2ccc(Cl)c(Cl)c2)c([N+](=O)[O-])c1]

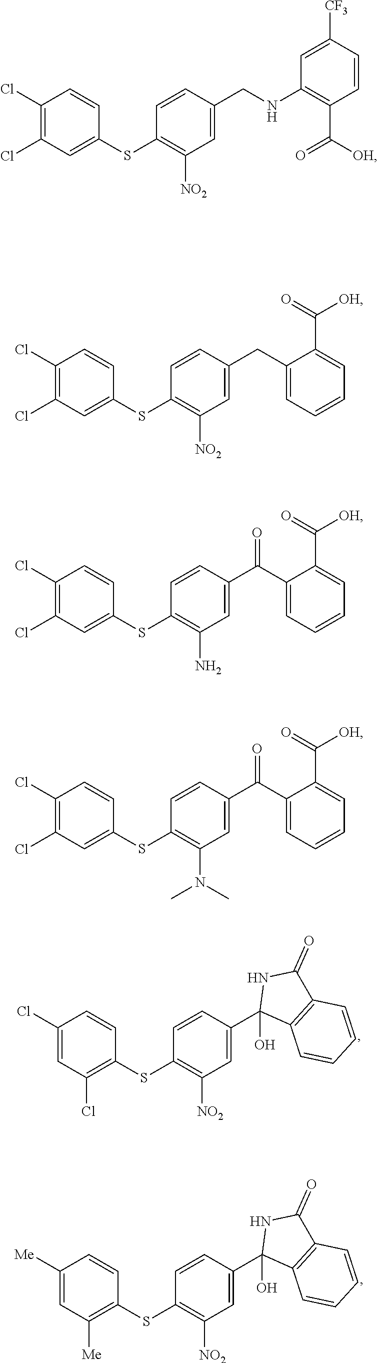 [SMILES: O=C(O)c1ccc(C(F)(F)F)cc1NCc1ccc(Sc2ccc(Cl)c(Cl)c2)c([N+](=O)[O-])c1 ; O=C(O)c1ccccc1Cc1ccc(Sc2ccc(Cl)c(Cl)c2)c([N+](=O)[O-])c1 ; Nc1cc(C(=O)c2ccccc2C(=O)O)ccc1Sc1ccc(Cl)c(Cl)c1 ; CN(C)c1cc(C(=O)c2ccccc2C(=O)O)ccc1Sc1ccc(Cl)c(Cl)c1 ; O=C1NC(O)(c2ccc(Sc3ccc(Cl)cc3Cl)c([N+](=O)[O-])c2)c2ccccc21 ; Cc1ccc(Sc2ccc(C3(O)NC(=O)c4ccccc43)cc2[N+](=O)[O-])c(C)c1]

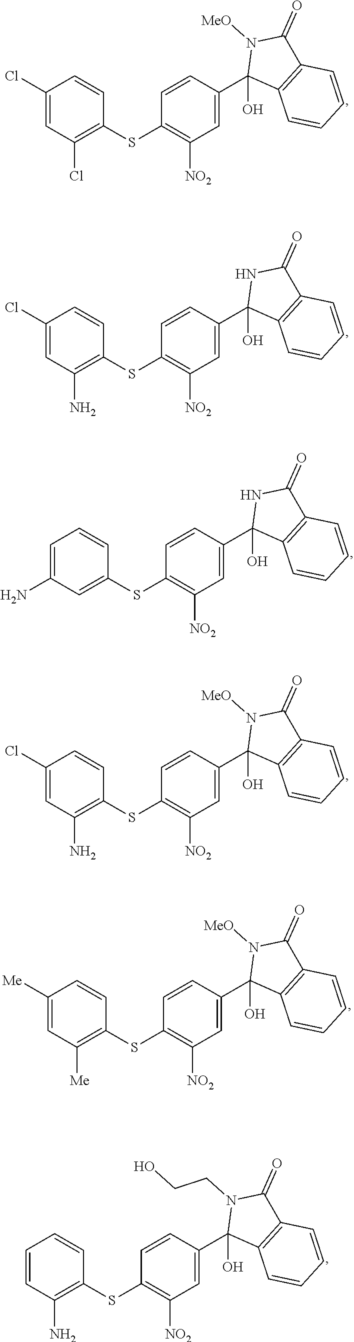 [SMILES: CON1C(=O)c2ccccc2C1(O)c1ccc(Sc2ccc(Cl)cc2Cl)c([N+](=O)[O-])c1 ; Nc1cc(Cl)ccc1Sc1ccc(C2(O)NC(=O)c3ccccc32)cc1[N+](=O)[O-] ; Nc1cccc(Sc2ccc(C3(O)NC(=O)c4ccccc43)cc2[N+](=O)[O-])c1 ; CON1C(=O)c2ccccc2C1(O)c1ccc(Sc2ccc(Cl)cc2N)c([N+](=O)[O-])c1 ; CON1C(=O)c2ccccc2C1(O)c1ccc(Sc2ccc(C)cc2C)c([N+](=O)[O-])c1 ; Nc1ccccc1Sc1ccc(C2(O)c3ccccc3C(=O)N2CCO)cc1[N+](=O)[O-]]

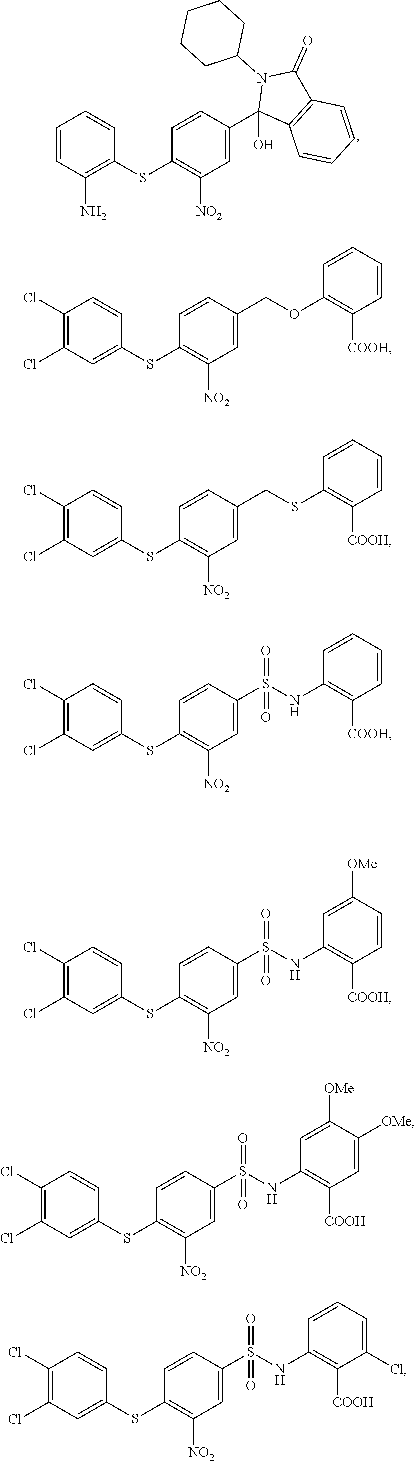

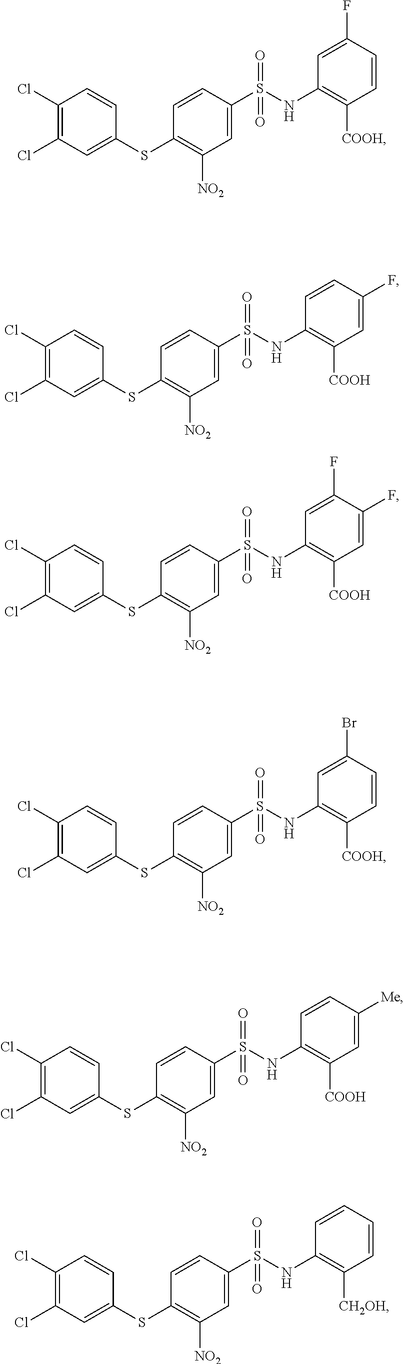 [SMILES: O=C(O)c1ccc(F)cc1NS(=O)(=O)c1ccc(Sc2ccc(Cl)c(Cl)c2)c([N+](=O)[O-])c1 ; O=C(O)c1cc(F)ccc1NS(=O)(=O)c1ccc(Sc2ccc(Cl)c(Cl)c2)c([N+](=O)[O-])c1 ; O=C(O)c1cc(F)c(F)cc1NS(=O)(=O)c1ccc(Sc2ccc(Cl)c(Cl)c2)c([N+](=O)[O-])c1 ; O=C(O)c1ccc(Br)cc1NS(=O)(=O)c1ccc(Sc2ccc(Cl)c(Cl)c2)c([N+](=O)[O-])c1 ; Cc1ccc(NS(=O)(=O)c2ccc(Sc3ccc(Cl)c(Cl)c3)c([N+](=O)[O-])c2)c(C(=O)O)c1 ; O=[N+]([O-])c1cc(S(=O)(=O)Nc2ccccc2CO)ccc1Sc1ccc(Cl)c(Cl)c1]

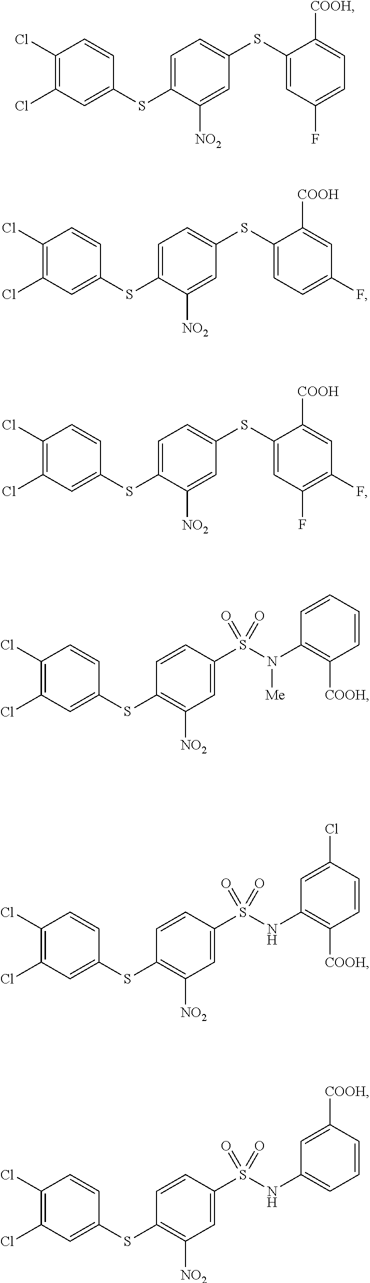 [SMILES: O=C(O)c1ccc(F)cc1Sc1ccc(Sc2ccc(Cl)c(Cl)c2)c([N+](=O)[O-])c1 ; O=C(O)c1cc(F)ccc1Sc1ccc(Sc2ccc(Cl)c(Cl)c2)c([N+](=O)[O-])c1 ; O=C(O)c1cc(F)c(F)cc1Sc1ccc(Sc2ccc(Cl)c(Cl)c2)c([N+](=O)[O-])c1 ; CN(c1ccccc1C(=O)O)S(=O)(=O)c1ccc(Sc2ccc(Cl)c(Cl)c2)c([N+](=O)[O-])c1 ; O=C(O)c1ccc(Cl)cc1NS(=O)(=O)c1ccc(Sc2ccc(Cl)c(Cl)c2)c([N+](=O)[O-])c1 ; O=C(O)c1cccc(NS(=O)(=O)c2ccc(Sc3ccc(Cl)c(Cl)c3)c([N+](=O)[O-])c2)c1]

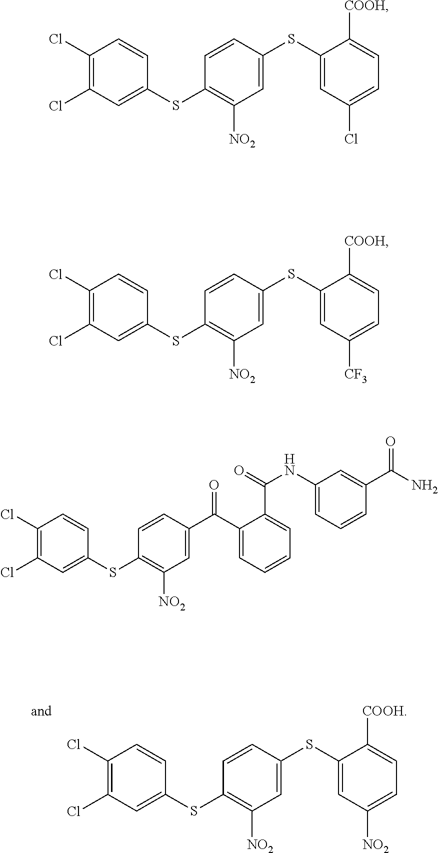 [SMILES: O=C(O)c1ccc(Cl)cc1Sc1ccc(Sc2ccc(Cl)c(Cl)c2)c([N+](=O)[O-])c1 ; O=C(O)c1ccc(C(F)(F)F)cc1Sc1ccc(Sc2ccc(Cl)c(Cl)c2)c([N+](=O)[O-])c1 ; NC(=O)c1cccc(NC(=O)c2ccccc2C(=O)c2ccc(Sc3ccc(Cl)c(Cl)c3)c([N+](=O)[O-])c2)c1 ; O=C(O)c1ccc([N+](=O)[O-])cc1Sc1ccc(Sc2ccc(Cl)c(Cl)c2)c([N+](=O)[O-])c1]

In certain embodiments, the compounds described herein are isotopically-labelled compounds, which are identical in chemical structure, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. The compounds described herein that contain the aforementioned isotopes and/or other isotopes of other atoms are within the scope of this disclosure. Certain isotopically-labelled compounds described herein, for example those into which radioactive isotopes such as ³H and ¹⁴C are incorporated, can be useful in drug and/or substrate tissue distribution assays. Tritiated, i.e, ³H, and carbon-14, i.e., ¹⁴C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds described herein can generally be prepared by carrying out the procedures disclosed in the Examples below, by substituting a readily available isotopically labelled reagent for a non-isotopically labelled reagent.

The compounds described herein may contain one or more asymmetric centers and therefore exist in different enantiomeric or diasteromeric forms. This disclosure contemplates the all optical isomers and stereoisomers of the compounds described herein and mixtures thereof.

In certain embodiments, the compounds described herein may have asymmetric carbon atoms. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers, are considered as part of the invention.

Also provided herein are pharmaceutical compositions containing, and methods of treating bacterial infections through administering, prodrugs of compounds described herein. Compounds described herein having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds described herein. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The compounds described herein that are basic in nature are capable of forming a wide variety of salts with various inorganic and organic acids.

The compounds described herein that are acidic in nature are capable of forming base salts with various pharmacologically acceptable cations. Examples of such salts include the alkali metal or alkaline-earth metal salts and particularly, the sodium and potassium salts. These salts may be prepared by conventional techniques. The chemical bases which are used as reagents to prepare the pharmaceutically acceptable base salts of this invention are those which form non-toxic base salts with any acidic compounds described herein. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

In certain embodiments, the compounds described herein, and the pharmaceutically acceptable salts and solvates thereof (hereinafter “the active compounds”), may be administered through oral, parenteral, topical, or rectal routes in the treatment or prevention of bacterial or protozoa infections. Variations may nevertheless occur depending upon the species of mammal, fish or bird being treated and its individual response to said medicament, as well as on the type of pharmaceutical formulation chosen and the time period and interval at which such administration is carried out.

The present disclosure also provides a pharmaceutical composition comprising any one of the compounds described herein and at least one pharmaceutically acceptable excipient.

The compounds described herein and their pharmaceutically acceptable salts can be administered to a subject either alone or in combination with pharmaceutically acceptable carriers or diluents in a pharmaceutical composition according to standard pharmaceutical practice. The compounds can be administered orally or parenterally, preferably parenterally. Parenteral administration includes intravenous, intramuscular, intraperitoneal, subcutaneous and topical. In certain embodiments, the compounds described herein are administered intravenously.

Accordingly, the present disclosure provides pharmaceutically acceptable compositions, which comprise a therapeutically-effective amount of one or more of the compounds described herein, formulated together with one or more pharmaceutically acceptable carriers (additives) and/or diluents. The pharmaceutical compositions of the present disclosure may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue.

As used herein, unless otherwise indicated, the phrase “pharmaceutically acceptable salt(s)” includes salts of acidic or basic groups which may be present in the compounds described herein. The compounds described herein that contain basic groups, such as amines, are capable of forming a wide variety of salts with various inorganic and organic acids. The acids that may be used to prepare pharmaceutically acceptable acid addition salts of such basic compounds described herein are those that form relatively non-toxic acid addition salts, i.e., salts containing pharmacologically acceptable anions, such as acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

In other cases, the compounds described herein may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

Methods of preparing these formulations include the step of bringing into association a compound described herein with the carrier and, optionally, one or more accessory ingredients. In general, the formulations are prepared by uniformly and intimately bringing into association a compound described herein with liquid carriers (liquid formulation), liquid carriers followed by lyophylization (powder formulation for reconstitution with sterile water or the like), or finely divided solid carriers, or both, and then, if necessary, shaping or packaging the product.

Pharmaceutical compositions of the present disclosure suitable for parenteral administration comprise one or more compounds described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents. In the examples, the active ingredients are brought together with the pharmaceutically acceptable carriers in solution and then lyophilized to yield a dry powder. The dry powder is packaged in unit dosage form and then reconstituted for parental administration by adding a sterile solution, such as water or normal saline, to the powder.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the compounds described herein may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

In one embodiment, the active compounds may be administered alone or in combination with pharmaceutically acceptable carriers or diluents by the routes previously indicated, and such administration may be carried out in single or multiple doses. More particularly, the active compounds may be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 5.0% to about 70% by weight.

In one embodiment, for oral administration, tablets containing various excipients such as microcrystalline cellulose, sodium citrate, calcium carbonate, dicalcium phosphate and glycine may be employed along with various disintegrants such as starch (and preferably corn, potato, or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

In one embodiment, for parenteral administration, solutions of an active compound in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques will be known to those skilled in the art.

In certain embodiments, it is also possible to administer the active compounds of the present invention topically and this may be done by way of creams, jellies, gels, pastes, patches, ointments and the like, in accordance with standard pharmaceutical practice.

In certain embodiments, for administration to animals other than humans, such as cattle or domestic animals, the active compounds may be administered in the feed of the animals or orally as a drench composition.

In certain embodiments, the active compounds may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

In certain embodiments, the active compounds may also be coupled with soluble polymers as targetable drug carriers. Such polymers can include polyvinylpyrrolidone, pyran copolymer, polyhydroxypropylmethacrylamide phenyl, polyhydroxyethylaspartamide-phenol, or polyethyleneoxide-polylysine substituted with palmitoylresidues. Furthermore, the active compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

In another aspect, provided herein is a method of treating a bacterial infection in a subject in need thereof, the method comprising the step of administering a therapeutically effective amount of a compound described herein to the subject.

In certain embodiments, the subject a canine, feline, bovine, equine, non-human primate, or human. In certain embodiments, the subject is a human.

The compounds described herein can be used in the treatment of Gram-positive bacteria. In certain embodiments, the bacteria is selected from the group consisting of Streptococcus agalactiae, Staphylococcus aureus, Staphylococcus epidermidis, Streptococcus pneumoniae, Streptococcus pyogenes, Staphylococcus saprophyticus, and Enterococcus faecium.

In certain embodiments, the compounds described herein are bactericidal or bacteriostatic.

Provided herein is a method for treating a bacterial infection in a subject in need thereof, comprising the step of administering a therapeutically effective amount of a compound described herein to the subject. Exemplary bacterial infections include, but are not limited to, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Enterococcus faecalis, E. faecium, E. casselflavus, S. epidermidis, S. haemolyticus, or Peptostreptococcus spp.; pharyngitis, rheumatic fever, and glomerulonephritis related to infection by Streptococcus pyogenes, Groups C and G streptococci, Corynebacterium diphtheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; blood and tissue infections, including endocarditis and osteomyelitis, caused by S. aureus, S. haemolyticus, E. faecalis, E. faecium, E. durans, including strains resistant to known antibacterials such as, but not limited to, beta-lactams, vancomycin, aminoglycosides, quinolones, chloramphenicol, tetracylines and macrolides; uncomplicated skin and soft tissue infections and abscesses, and puerperal fever related to infection by Staphylococcus aureus, coagulase-negative staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus aureus, coagulase-negative staphylococcal species, or Enterococcus spp.; urethritis and cervicitis; sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; infections caused by Mycobacterium tuberculosis, M. leprae, M. paratuberculosis, M. kansasii, or M. chelonei; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis or cardiovascular disease related to infection by Helicobacter pylori or Chlamydia pneumoniae. Bacterial infections and disorders related to such infections, which may be treated or prevented in animals include the following: bovine respiratory disease related to infection by P. haemolytica, P. multocida, Mycoplasma bovis, or Bordetella spp.; cow enteric disease related to infection by E. coli or protozoa (i.e., coccidia, cryptosporidia, etc.); dairy cow mastitis related to infection by S. aureus, Strep. uberis, Streptococcus agalactiae, Streptococcus dysgalactiae, Klebsiella spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodysinteriae; cow footrot related to infection by Fusobacterium spp.; cow metritis related to infection by E coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and disorders related to such infections, which may be treated or prevented in accord with the method of the present invention are referred to in J. P. Sanford et al., “The Sanford Guide To Antimicrobial Therapy,” 26 th Edition, (Antimicrobial Therapy, Inc., 1996).

In another aspect, provided herein is the use of a compound described herein for the preparation of a medicament for the treatment of a bacterial infection. In certain embodiments, the bacterial infection is the result of Gram-positive bacteria. Exemplary Gram-positive bacteria include, but are not limited to, Streptococcus agalactiae, Staphylococcus aureus, Staphylococcus epidermidis, Streptococcus pneumoniae, Streptococcus pyogenes, Staphylococcus saprophyticus, and Enterococcus faecium.

In another aspect, provided herein is a method of treating a bacterial infection in a subject in need thereof, the method comprising the step of co-administering a therapeutically effective amount of a compound described herein and an antibacterial agent to the subject. The antibacterial agent can be any antibacterial agent known in the art.

### Examples

Using this improved pharmacophore, we undertook the identification of a drug lead with good solubility and appropriate drug-like properties18 through in silico screening of publicly available compound libraries. After screening the mini-Maybridge library (53,000 compounds; http://www.maybridge.com/), 27 hits were identified, from which 7 that demonstrated the appropriate distance constraints to the β′-CH region and energy-minimized conformations were short-listed for further investigation.

All seven compounds were initially tested in an ELISA-based binding assay using σ^{A} and a GST-tagged β′ subunit fragment that encompasses the CH region (β′-CH).¹⁴ Three of the seven compounds including compound C3 possessed inhibitory activity against the binding of σ^{A} and β′ fragments.

C3 derivatives were synthesized and antimicrobial activity measured against Streptococcus pneumoniae (MIC, minimum inhibitory concentration).

TABLE 1

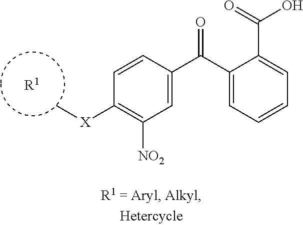

| | | | MIC |
|---|---|---|---|
| Compd. | R¹ | X | μg/mL |
| C3-1 | 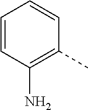 | S | 256 |
| C3-2 | 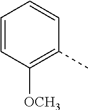 | S | 128 |
| C3-3 | 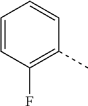 | S | 128 |
| C3-4 | 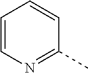 | S | >256 |
| C3-5 | 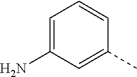 | S | 64 |
| C3-6 | 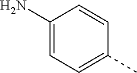 | S | 128 |
| C3-7 | 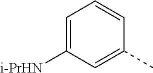 | S | 32 |
| C3-8 | 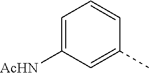 | S | 256 |
| C3-9 | 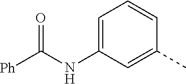 | S | 32 |
| C3-10 | 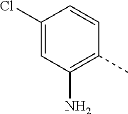 | S | 64 |
| C3-11 | 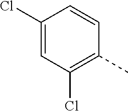 | S | 32 |
| C3-12 | 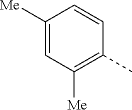 | S | 32 |
| C3-13 | 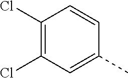 | S | 8 |
| C3-14 | 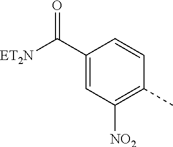 | S | 256 |
| C3-15 | 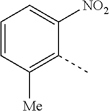 | S | 128 |
| C3-16 | 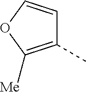 | S | 256 |
| C3-17 | 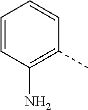 | NH | >256 |
| C3-18 | 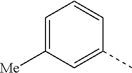 | NH | 128 |
| C3-19 | 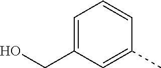 | NH | >256 |
| C3-20 | 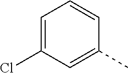 | NH | |
| C3-21 | 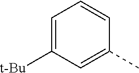 | NH | 256 |
| C3-22 | 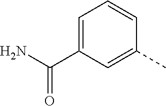 | NH | >256 |
| C3-23 | 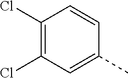 | NH | 64 |
| C3-24 | 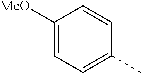 | NH | >256 |
| C3-25 | 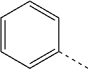 | NH | 256 |
| C3-26 | 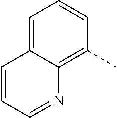 | NH | >256 |
| C3-27 | 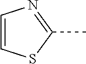 | NH | >256 |
| C3-28 | 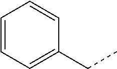 | NH | 256 |
| C3-29 | 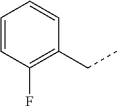 | NH | 256 |
| C3-30 | 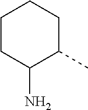 | NH | >256 |
| C3-31 | 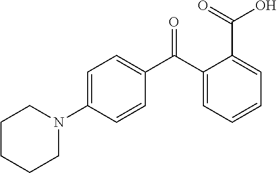 | | >256 |
| C3-32 | 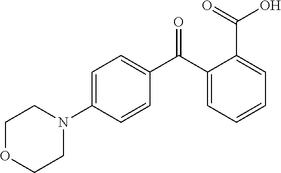 | | >256 |

TABLE 2

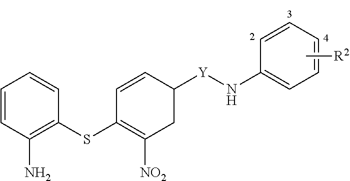

| | | | MIC |
|---|---|---|---|
| Compd. | Y | R² | μg/mL |
| C3-33 C3-34 C3-35 | 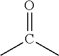 | 2-COOH 3-COOH 4-COOH | 64 128 256 |
| C3-36 | 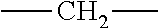 | 2-COOH | 64 |
| C3-37 | | 3-COOH | 64 |
| C3-38 | | 4-COOH | 256 |

TABLE 3

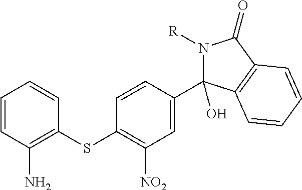

| | | MIC |
|---|---|---|
| Cmpd. | R | μg/mL |
| C3-39 | H | 32 |
| C3-40 | 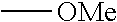 | 32 |
| C3-41 | 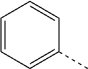 | >256 |
| C3-42 | 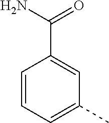 | 32 |
| C3-43 | 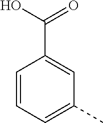 | 64 |
| C3-44 | 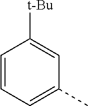 | >256 |
| C3-45 | 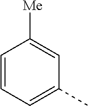 | >256 |
| C3-46 | 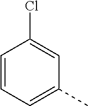 | >256 |
| C3-47 | 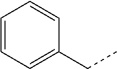 | >256 |
| C3-48 | 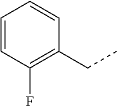 | >256 |

TABLE 4

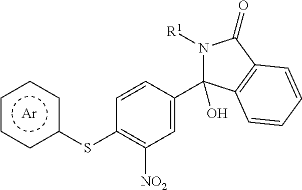

| | | | MIC |
|---|---|---|---|
| Compd. | Ar | R | μg/mL |
| C3-49 | 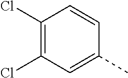 | H | 4 |
| C3-50 | 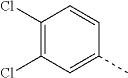 | OMe | 4 |

Materials and Methods

Bacterial Strains was used in this study is Streptococcus pneumoniae (Klein) Chester (ATCC® 49619™)

### Examples

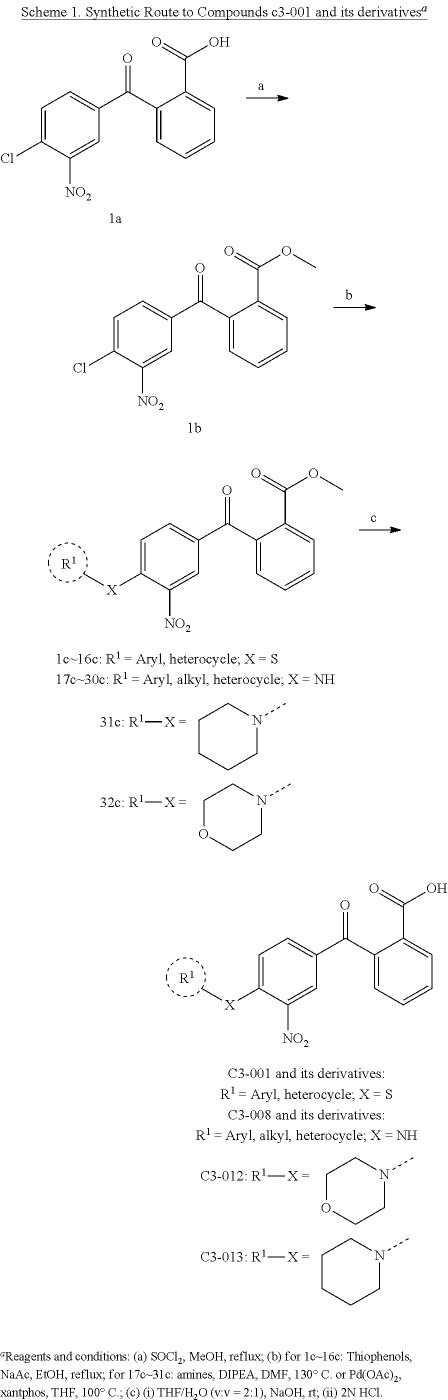

### Synthesis of methyl 2-(4-chloro-3-nitrobenzoyl)benzoate (1b)

A solution of 2-(4-chloro-3-nitrobenzoyl) benzoic acid 1a (3.057 g, 10 mmol) in MeOH was cooled to 0° C. followed by a dropwise addition of thionyl chloride (0.5 ml). The mixture was refluxed for 24 h. After evaporation of the volatiles, the residue was treated with 5 ml MeOH and stirred at rt. for 10 min. The precipitate was collected by filtration and dried in vacuum to give compound 1b as a white solid (2.877 g, 90%).

General Procedure for the Synthesis of Compound 1c-16c

To a flask was added compound 1b (64 mg, 0.2 mmol), benzenethiol (1.2 equiv, 0.24 mmol), NaAc (82 mg, 1 mmol.) and EtOH 5 ml. The mixture was heated to reflux for 4 h. After cooling to rt., the precipitate was collected via filtration and washed with appropriate EtOH and water successively and dried in vacuum to give the titled compounds. Otherwise, water was added and then the aqueous was extracted by EA. The combined organic layers were dried over Na₂SO₄ and purification by chromatography to provide the titled compounds.

General Procedure for the Synthesis of 17c-32c

Two different procedures were used to obtain the title compounds:

Method A To a flask was added compound 1b (64 mg, 0.2 mmol), amine (2 equiv, 0.4 mmol), DIPEA (99 μl, 0.6 mmol.) and DMF 2 ml. The mixture was heated to reflux for 6 h. After cooling to ambient, water was added and then the aqueous was extracted by EA. The combined organic layers were dried over Na₂SO₄ and purification by chromatography to provide the titled compounds.

Method B To an oven-dried Schlenk tube was charged with compound 1b (64 mg, 0.3 mmol), amine (1.1 equiv, 0.33 mmol), Pd(OAc)₂ (0.09 mmol, 20.2 mg), Xant-phos (0.09 mmol, 52.1 mg) Cs₂CO₃ (0.9 mmol, 293 mg) and THF 5 ml, and purged with nitrogen. The reaction mixture was heated to 100° C. for 12 h and then cooled to ambient. Water was added and then the aqueous was extracted by EA. The combined organic layers were dried over Na₂SO₄ and purification by chromatography to provide the titled compounds.

General Procedure for the Synthesis of C3-001 and Related Analogs

The methyl esters of the title compounds (1c-32c) were hydrolyzed with 1 M NaOH in TFH (1:1) at room temperature overnight. The mixture was then diluted with a small amount of water and washed twice with CH₂Cl₂. The aqueous solution was acidified by the addition of 2 M HCl. The precipitate was collected by filtration and washed with water to afford the title compounds. If the compound was not pure at this stage of the procedure, it was purified by silica gel column.

### 2-(4-((2-Aminophenyl)thio)-3-nitrobenzoyl)benzoic Acid (C3-001)

The title compound was prepared from the hydrolysis of 1c (60 mg, 0.15 mmol) in 1N NaOH (0.8 ml) and THF (0.8 ml). Yellow solid, 50 mg, 85% yield, mp 217-219° C. ¹H NMR (400 MHz, DMSO) δ 13.30 (s, 1H), 8.33 (d, J=1.4 Hz, 1H), 8.02 (d, J=7.6 Hz, 1H), 7.82-7.72 (m, 2H), 7.69 (t, J=7.2 Hz, 1H), 7.44 (d, J=7.3 Hz, 1H), 7.35 (d, J=7.5 Hz, 1H), 7.28 (t, J=7.3 Hz, 1H), 6.90 (d, J=8.6 Hz, 1H), 6.83 (d, J=8.1 Hz, 1H), 6.65 (t, J=7.3 Hz, 1H), 5.62 (s, 2H). ¹³C NMR (101 MHz, DMSO) δ 167.4, 151.4, 145.1, 143.1, 141.2, 137.7, 134.8, 133.3, 132.9, 132.9, 130.9, 130.5, 130.2, 127.8, 127.4, 126.1, 117.3, 115.7, 109.1. HRMS (ESI): calcd for C₂₀H₁₃N₂O₅S, (M−H)⁻ 393.0551, found 393.0547. HPLC purity: 97.00%.

### 2-(4-((2-Methoxyphenyl)thio)-3-nitrobenzoyl)benzoic Acid (C3-004)

The title compound was prepared from the hydrolysis of 2c (70 mg, 0.16 mmol) in 1N NaOH (0.8 ml) and THF (0.8 ml). Yellow solid, 60 mg, 92% yield, mp 209-211° C. ¹H NMR (400 MHz, DMSO) δ 13.31 (s, 1H), 8.34 (d, J=1.7 Hz, 1H), 8.01 (d, J=7.5 Hz, 1H), 7.75 (dd, J=13.3, 7.2 Hz, 2H), 7.69 (m, 1H), 7.64 (m, 2H), 7.47 (d, J=7.3 Hz, 1H), 7.26 (d, J=8.2 Hz, 1H), 7.13 (t, J=7.3 Hz, 1H), 6.88 (d, J=8.5 Hz, 1H), 3.77 (s, 3H). ¹³C NMR (101 MHz, DMSO) δ 194.5, 167.3, 160.5, 144.4, 143.3, 140.8, 137.7, 134.6, 133.8, 133.5, 133.3, 130.7, 130.3, 128.4, 127.8, 126.0, 122.4, 116.4, 113.2, 56.5. HRMS (ESI): calcd for C₂₁H₁₄NO₆S, (M−H)⁻ 408.0547, found 408.0539. HPLC purity: 97.04%.

### 2-(4-((2-Fluorophenyl)thio)-3-nitrobenzoyl)benzoic Acid (C3-051)

The title compound was prepared from the hydrolysis of 3c (60 mg, 0.15 mmol) in 1N NaOH (0.7 ml) and THF (0.7 ml). Yellow solid, 43 mg, 72% yield, mp 175-176° C. ¹H NMR (400 MHz, DMSO) δ 13.34 (s, 1H), 8.39 (s, 1H), 8.02 (d, J=7.5 Hz, 1H), 7.76 (t, J=7.6 Hz, 3H), 7.70 (dd, J=13.9, 6.6 Hz, 2H), 7.49 (t, J=8.3 Hz, 2H), 7.42 (t, J=7.4 Hz, 1H), 6.96 (d, J=8.4 Hz, 1H). ¹³C NMR (101 MHz, DMSO) δ 194.4, 168.2 (d, J=8.1 Hz), 164.1, 161.6 (d, J=248.5 Hz), 144.5, 140.7, 139.9 (d, J=3.0 Hz), 138.4 (d, J=10.1 Hz), 138.2, 137.2 (d, J=3.0 Hz), 134.5 (d, J=8.1 Hz), 133.8, 130.2, 129.8, 129.5, 128.0, 126.9 (d, J=3.0 Hz), 126.3, 125.4, 117.5 (d, J=22.2 Hz), 116.7 (d, J=19.2 Hz). HRMS (ESI): calcd for C₂₀H₁₁FNO₅S, (M−H)⁻ 396.0347, found 396.0345. HPLC purity: 99.25%.

### 2-(3-Nitro-4-(pyridin-2-ylthio)benzoyl)benzoic Acid (C3-003)

The title compound was prepared from the hydrolysis of 4c (70 mg, 0.18 mmol) in 1N NaOH (0.9 ml) and THF (0.9 ml). Yellow solid, 40 mg, 58% yield, mp 205-206° C. ¹H NMR (400 MHz, DMSO) δ 13.74 (s, 1H), 8.62 (d, J=4.0 Hz, 1H), 8.31 (d, J=1.7 Hz, 1H), 8.02 (d, J=7.5 Hz, 1H), 7.92 (t, J=7.0 Hz, 1H), 7.77 (m, 2H), 7.70 (m, 2H), 7.49 (m, 2H), 7.37 (d, J=8.4 Hz, 1H). ¹³C NMR (101 MHz, DMSO) δ 194.5, 167.3, 153.6, 151.6, 147.1, 140.7, 139.1, 138.8, 136.0, 133.3, 132.0, 130.8, 130.5, 130.1, 128.8, 128.8, 127.9, 125.4, 124.5. HRMS (ESI): calcd for C₁₉H₁₁N₂O₅S, (M−H)⁻ 379.0394, found 379.0388. HPLC purity: 100.00%.

### 2-(4-((3-Aminophenyl)thio)-3-nitrobenzoyl)benzoic Acid (C3-031)

The title compound was prepared from the hydrolysis of 5c (60 mg, 0.15 mmol) in 1N NaOH (0.7 ml) and THF (0.7 ml). Yellow solid, 40 mg, 69% yield, mp 123-125° C. ¹H NMR (400 MHz, DMSO) δ 13.31 (s, 1H), 8.31 (d, J=1.1 Hz, 1H), 8.02 (d, J=7.4 Hz, 1H), 7.85-7.73 (m, 2H), 7.69 (t, J=7.4 Hz, 1H), 7.46 (d, J=7.2 Hz, 1H), 7.20 (t, J=7.7 Hz, 1H), 7.08 (d, J=8.5 Hz, 1H), 6.79 (s, 1H), 6.74 (t, J=8.7 Hz, 2H), 5.53 (s, 2H). ¹³C NMR (101 MHz, DMSO) δ 194.5, 167.2, 151.1, 144.8, 144.4, 140.7, 134.5, 133.6, 133.2, 131.5, 130.7, 130.4, 130.2, 129.3, 128.9, 127.9, 126.0, 122.4, 120.1, 116.5. HRMS (ESI): calcd for C₂₀H₁₃N₂O₅S, (M−H)⁻ 393.0551, found 393.0544. HPLC purity: 95.24%.

### 2-(4-((4-Aminophenyl)thio)-3-nitrobenzoyl)benzoic Acid (C3-032)

The title compound was prepared from the hydrolysis of 6c (80 mg, 0.2 mmol) in 1N NaOH (1.0 ml) and THF (1.0 ml). Yellow solid, 60 mg, 76% yield, mp 129-131° C. ¹H NMR (400 MHz, DMSO) δ 13.31 (s, 1H), 8.29 (d, J=1.5 Hz, 1H), 8.00 (d, J=7.5 Hz, 1H), 7.80-7.71 (m, 2H), 7.67 (t, J=7.3 Hz, 1H), 7.44 (d, J=7.3 Hz, 1H), 7.23 (d, J=8.4 Hz, 2H), 6.98 (d, J=8.6 Hz, 1H), 6.69 (d, J=8.5 Hz, 2H), 5.79 (s, 2H). ¹³C NMR (101 MHz, DMSO) δ 194.3, 167.3, 151.8, 146.9, 143.9, 141.1, 137.6, 134.36, 133.39, 133.1, 130.6, 130.4, 130.2, 128.4, 127.6, 125.9, 115.7, 111.8. HRMS (ESI): calcd for C₂₀ H₁₃N₂O₅S, (M−H)⁻ 393.0551, found 393.0548. HPLC purity: 99.73%.

### 2-(4-((3-(Isopropylamino)phenyl)thio)-3-nitrobenzoyl)benzoic Acid (C3-052)

The title compound was prepared from the hydrolysis of 7c (65 mg, 0.14 mmol) in 1N NaOH (0.7 ml) and THF (0.7 ml). Yellow solid, 40 mg, 66% yield, mp 130-132° C. ¹H NMR (400 MHz, DMSO) δ 13.32 (s, 1H), 8.31 (s, 1H), 8.01 (d, J=7.5 Hz, 1H), 7.80 (d, J=8.7 Hz, 1H), 7.75 (d, J=7.5 Hz, 1H), 7.69 (t, J=7.5 Hz, 1H), 7.46 (d, J=7.3 Hz, 1H), 7.25 (t, J=7.7 Hz, 1H), 7.08 (d, J=8.5 Hz, 1H), 6.78-6.69 (m, 3H), 5.86 (s, 1H), 3.53 (m, 1H), 1.12 (d, J=6.2 Hz, 6H). ¹³C NMR (101 MHz, DMSO) δ 194.5, 167.3, 150.3, 144.8, 144.3, 140.8, 134.4, 133.6, 133.2, 131.5, 130.7, 130.5, 130.2, 129.5, 128.8, 127.9, 126.0, 121.8, 118.6, 114.9, 43.4, 22.7. HRMS (ESI): calcd for C₂₃H₁₉N₂O₅S, (M−H)⁻ 435.102, found 435.1022. HPLC purity: 97.60%.

### 2-(4-((3-Acetamidophenyl)thio)-3-nitrobenzoyl)benzoic Acid (C3-070)

The title compound was prepared from the hydrolysis of 8c (45 mg, 0.1 mmol) in 1N NaOH (0.5 ml) and THF (0.5 ml). Yellow solid, 32 mg, 73% yield, mp 144-146° C. ¹H NMR (400 MHz, DMSO) δ 13.33 (s, 1H), 10.21 (s, 1H), 8.34 (s, 1H), 8.01 (d, J=7.5 Hz, 1H), 7.94 (s, 1H), 7.80-7.65 (m, 4H), 7.51 (t, J=7.9 Hz, 1H), 7.46 (d, J=7.3 Hz, 1H), 7.31 (d, J=7.5 Hz, 1H), 6.99 (d, J=8.5 Hz, 1H), 2.05 (s, 3H). ¹³C NMR (101 MHz, DMSO) δ 194.4, 169.2, 167.3, 144.5, 143.7, 141.60, 141.0, 134.9, 133.8, 133.3, 131.4, 130.7, 130.2, 130.0, 129.7, 128.9, 127.7, 125.8, 125.5, 121.4, 24.5. HRMS (ESI): calcd for C₂₂H₁₅N₂O₆S, (M−H)⁻ 435.0656, found 435.0651. HPLC purity: 96.56%.

### 2-(4-((3-Benzamidophenyl)thio)-3-nitrobenzoyl)benzoic Acid (C3-062)

The title compound was prepared from the hydrolysis of 9c (57 mg, 0.1 mmol) in 1N NaOH (0.5 ml) and THF (0.5 ml). Yellow solid, 31 mg, 57% yield, mp 144-145° C. ¹H NMR (400 MHz, DMSO) δ 13.31 (s, 1H), 10.49 (s, 1H), 8.35 (d, J=1.6 Hz, 1H), 8.15 (s, 1H), 8.01 (d, J=7.8 Hz, 2H), 7.95 (d, J=7.3 Hz, 2H), 7.79 (d, J=9.3 Hz, 1H), 7.75 (d, J=7.2 Hz, 1H), 7.69 (t, J=7.2 Hz, 1H), 7.62 (d, J=7.3 Hz, 1H), 7.60-7.51 (m, 3H), 7.47 (d, J=7.1 Hz, 1H), 7.39 (d, J=7.5 Hz, 1H), 7.06 (d, J=8.5 Hz, 1H). ¹³C NMR (101 MHz, DMSO) δ 194.5, 167.2, 166.3, 144.6, 143.8, 141.5, 140.7, 135.0, 134.8, 133.9, 133.3, 132.3, 131.4, 130.9, 130.8, 130.5, 130.1, 129.7, 129.0, 128.9, 128.2, 127.8, 126.9, 125.9, 122.7. HRMS (ESI): calcd for C₂₇H₁₇N₂O₆S, (M−H)⁻ 497.0813, found 497.0805. HPLC purity: 95.26%.

### 2-(4-((2-Amino-4-chlorophenyl)thio)-3-nitrobenzoyl)benzoic Acid (C3-063)

The title compound was prepared from the hydrolysis of 10c (70 mg, 0.16 mmol) in 1N NaOH (0.8 ml) and THF (0.8 ml). Yellow solid, 41 mg, 60% yield, mp 118-120° C. ¹H NMR (400 MHz, DMSO) δ 13.32 (s, 1H), 8.33 (d, J=1.6 Hz, 1H), 8.02 (d, J=7.5 Hz, 1H), 7.76 (dd, J=15.0, 7.8 Hz, 2H), 7.68 (t, J=7.2 Hz, 1H), 7.44 (d, J=7.3 Hz, 1H), 7.37 (d, J=8.2 Hz, 1H), 6.92 (d, J=8.5 Hz, 1H), 6.86 (d, J=2.0 Hz, 1H), 6.66 (dd, J=8.2, 2.1 Hz, 1H), 5.93 (s, 2H). ¹³C NMR (101 MHz, DMSO) δ 194.6, 167.3, 152.5, 145.3, 142.6, 141.0, 139.3, 137.4, 134.7, 133.6, 133.2, 130.6, 130.3, 127.9, 127.6, 126.04, 116.8, 114.6, 108.1. HRMS (ESI): calcd for C₂₀H₁₂ClN₂O₅S, (M−H)⁻ 427.0161, found 427.0152. HPLC purity: 100.000%.

### 2-(4-((2,4-Dichlorophenyl)thio)-3-nitrobenzoyl)benzoic Acid (C3-061)

The title compound was prepared from the hydrolysis of 11c (60 mg, 0.13 mmol) in 1N NaOH (0.6 ml) and THF (0.6 ml). Yellow solid, 52 mg, 89% yield, mp 260-262° C. ¹H NMR (400 MHz, DMSO) δ 13.40 (S, 1H), 8.39 (d, J=10.3 Hz, 1H), 8.01 (dd, J=18.5, 10.5 Hz, 2H), 7.87 (dd, J=11.7, 8.3 Hz, 1H), 7.82-7.60 (m, 4H), 7.49 (t, J=8.9 Hz, 1H), 6.92 (t, J=10.1 Hz, 1H). ¹³C NMR (101 MHz, DMSO) δ 194.4, 167.2, 144.7, 141.1, 140.7, 140.3, 139.8, 137.5, 135.3, 134.2, 133.4, 131.1, 130.8, 130.3, 130.0, 129.8, 128.7, 127.7, 126.0. HRMS (ESI): calcd for C₂₀H₁₀Cl₂NO₅S, (M−H)⁻ 445.9662, found 445.9656. HPLC purity: 98.13%.

### 2-(4-((2,4-Dimethylphenyl)thio)-3-nitrobenzoyl)benzoic Acid (C3-059)

The title compound was prepared from the hydrolysis of 12c (68 mg, 0.16 mmol) in 1N NaOH (0.8 ml) and THF (0.8 ml). Yellow solid, 38 mg, 58% yield, mp 240-242° C. ¹H NMR (400 MHz, DMSO) δ 8.34 (s, 1H), 8.00 (d, J=7.5 Hz, 1H), 7.72 (dd, J=6.0, 2.7 Hz, 2H), 7.66 (t, J=7.4 Hz, 1H), 7.49 (d, J=7.8 Hz, 1H), 7.44 (d, J=7.3 Hz, 1H), 7.32 (s, 1H), 7.20 (d, J=7.6 Hz, 1H), 6.77 (d, J=8.5 Hz, 1H), 2.36 (s, 3H), 2.25 (s, 3H). ¹³C NMR (101 MHz, DMSO) δ 167.5, 144.5, 143.4, 143.0, 141.8, 141.1, 137.2, 134.8, 133.8, 133.0, 132.8, 130.8, 130.6, 130.2, 129.2, 127.9, 127.6, 126.1, 125.1, 21.3, 20.4. HRMS (ESI): calcd for C₂₂H₁₆NO₅S, (M−H)⁻ 406.0755, found 406.0747. HPLC purity: 99.01%.

### 2-(4-((3,4-Dichlorophenyl)thio)-3-nitrobenzoyl)benzoic Acid (C3-005)

The title compound was prepared from the hydrolysis of 13c (80 mg, 0.17 mmol) in 1N NaOH (0.9 ml) and THF (0.9 ml). Yellow solid, 45 mg, 62% yield, mp 247-248° C. ¹H NMR (400 MHz, DMSO) δ 13.34 (s, 1H), 8.37 (s, 1H), 8.02 (d, J=13.0 Hz, 2H), 7.84 (d, J=8.3 Hz, 1H), 7.76 (dd, J=14.9, 7.7 Hz, 2H), 7.72-7.67 (m, 1H), 7.64 (d, J=8.0 Hz, 1H), 7.46 (d, J=6.8 Hz, 1H), 7.07 (d, J=8.4 Hz, 1H). ¹³C NMR (101 MHz, DMSO) δ 194.4, 167.2, 144.8, 142.7, 140.7, 137.4, 136.1, 135.0, 134.4, 134.1, 133.3, 132.9, 130.8, 130.7, 130.5, 130.1, 129.5, 127.8, 125.8. HRMS (ESI): calcd for C₂₀H₁₀Cl₂NO₅S, (M−H)⁻ 445.9662, found 445.9651. HPLC purity: 99.73%.

### 2-(4-((4-(Diethylcarbamoyl)-2-nitrophenyl)thio)-3-nitrobenzoyl)benzoic Acid (C3-033)

The title compound was prepared from the hydrolysis of 14c (37 mg, 0.07 mmol) in 1N NaOH (0.4 ml) and THF (0.4 ml). Yellow solid, 25 mg, 69% yield, mp 129-130° C. ¹H NMR (400 MHz, DMSO) δ 13.40 (s, 1H), 8.37 (d, J=1.7 Hz, 1H), 8.19 (s, 1H), 8.03 (d, J=7.0 Hz, 1H), 7.86-7.62 (m, 5H), 7.51 (d, J=7.5 Hz, 1H), 7.37 (d, J=8.2 Hz, 1H), 3.46 (s, 2H), 3.23 (s, 2H), 1.17 (s, 3H), 1.08 (s, 3H). ¹³C NMR (101 MHz, DMSO) δ 194.5, 167.3, 151.2, 147.2, 140.6, 139.8, 138.5, 136.8, 136.5, 134.0, 133.4, 132.6, 132.2, 130.9, 130.5, 130.2, 127.9, 127.6, 125.5, 123.8, 43.4, 14.4, 13.1. HRMS (ESI): calcd for C₂₅H₂₀N₃O₈S, (M−H)⁻ 522.0977, found 522.0960. HPLC purity: 97.21%.

### 2-(4-((2-Methyl-6-nitrophenyl)thio)-3-nitrobenzoyl)benzoic Acid (C3-006)

The title compound was prepared from the hydrolysis of 15c (70 mg, 0.15 mmol) in 1N NaOH (0.8 ml) and THF (0.8 ml). Yellow solid, 50 mg, 76% yield, mp 269-271° C. ¹H NMR (400 MHz, DMSO) δ 13.40 (s, 1H), 8.41 (d, J=1.6 Hz, 1H), 8.02 (d, J=7.6 Hz, 1H), 7.94 (d, J=6.9 Hz, 1H), 7.79 (m, 4H), 7.69 (t, J=7.4 Hz, 1H), 7.50 (d, J=7.3 Hz, 1H), 6.86 (d, J=8.5 Hz, 1H), 2.38 (s, 3H). ¹³C NMR (101 MHz, DMSO) δ 194.4, 167.3, 155.6, 146.5, 144.9, 140.7, 135.4, 135.3, 134.3, 133.4, 133.0, 130.8, 130.3, 130.0, 128.4, 127.8, 126.0, 122.7, 120.8, 20.9. HRMS (ESI): calcd for C₂₁H₁₃N₂O₇S, (M−H)⁻ 437.0449, found 437.0443. HPLC purity: 99.56%.

### 2-(4-((2-Methylfuran-3-yl)thio)-3-nitrobenzoyl)benzoic Acid (C3-060)

The title compound was prepared from the hydrolysis of 16c (45 mg, 0.11 mmol) in 1N NaOH (0.5 ml) and THF (0.5 ml). Yellow solid, 31 mg, 73% yield, mp 198-199° C. ¹H NMR (400 MHz, DMSO) δ 13.45 (s, 1H), 8.35 (s, 1H), 8.02 (d, J=7.5 Hz, 1H), 7.84 (d, J=1.3 Hz, 1H), 7.82 (d, J=7.6 Hz, 1H), 7.77 (t, J=7.3 Hz, 1H), 7.70 (t, J=7.4 Hz, 1H), 7.47 (d, J=7.3 Hz, 1H), 7.17 (d, J=8.5 Hz, 1H), 6.58 (s, 1H), 2.33 (s, 3H). ¹³C NMR (101 MHz, DMSO) δ 194.5, 167.3, 158.8, 144.7, 143.8, 143.2, 140.8, 134.85, 133.84, 133.4, 130.8, 130.4, 130.1, 128.4, 127.8, 126.1, 115.3, 105.7, 12.0. HRMS (ESI): calcd for C₁₉H₁₂NO₆S, (M−H)⁻ 382.0391, found 382.0387. HPLC purity: 98.13%.

### 2-(4-((2-Aminophenyl)amino)-3-nitrobenzoyl)benzoic Acid (C3-008)

The title compound was prepared from the hydrolysis of 17c (40 mg, 0.1 mmol) in 1N NaOH (0.5 ml) and THF (0.5 ml). Dark yellow solid, 34 mg, 90% yield, mp 201-202° C. ¹H NMR (400 MHz, DMSO) δ 8.34 (s, 1H), 8.02 (d, J=7.6 Hz, 1H), 7.75 (t, J=7.6 Hz, 2H), 7.68 (t, J=7.4 Hz, 1H), 7.44 (d, J=7.3 Hz, 1H), 7.38-7.32 (m, 1H), 7.27 (t, J=7.6 Hz, 1H), 6.90 (d, J=8.5 Hz, 1H), 6.84 (d, J=8.1 Hz, 1H), 6.65 (t, J=7.4 Hz, 1H), 5.61 (s, 2H). ¹³C NMR (101 MHz, DMSO) δ 167.3, 151.4, 145.2, 143.4, 137.7, 134.6, 133.4, 133.3, 132.9, 130.6, 130.3, 130.1, 127.9, 127.6, 126.1, 117.3, 115.7, 109.0. HRMS (ESI): calcd for C₂₀H₁₄N₃O₅, (M−H)⁻ 376.0939, found 376.0936. HPLC purity: 95.02%.

### 2-(3-Nitro-4-(m-tolylamino)benzoyl)benzoic Acid (C3-037)

The title compound was prepared from the hydrolysis of 18c (60 mg, 0.15 mmol) in 1N NaOH (0.8 ml) and THF (0.8 ml). Dark yellow solid, 38 mg, 66% yield, mp 90-92° C. ¹H NMR (400 MHz, DMSO) δ 13.23 (s, 1H), 9.87 (s, 1H), 8.23 (d, J=1.9 Hz, 1H), 8.01 (d, J=7.6 Hz, 1H), 7.78 (d, J=10.4 Hz, 1H), 7.73 (d, J=7.3 Hz, 1H), 7.67 (t, J=7.5 Hz, 1H), 7.43 (d, J=7.4 Hz, 1H), 7.37 (t, J=7.7 Hz, 1H), 7.22-7.10 (m, 4H), 2.34 (s, 3H). ¹³C NMR (101 MHz, DMSO) δ 193.8, 167.4, 145.9, 141.2, 139.7, 138.4, 135.6, 135.5, 132.9, 132.2, 130.4, 130.3, 123.0, 128.7, 127.8, 127.5, 126.6, 126.1, 122.7, 117.0, 21.4. HRMS (ESI): calcd for C₂₁H₁₅N₂O₅, (M−H)⁻ 375.0986, found 375.0985. HPLC purity: 97.89%.

### 2-(4-((3-(Hydroxymethyl)phenyl)amino)-3-nitrobenzoyl)benzoic Acid (C3-009)

The title compound was prepared from the hydrolysis of 19c (50 mg, 0.12 mmol) in 1N NaOH (0.6 ml) and THF (0.6 ml). Dark yellow solid, 36 mg, 75% yield, mp 132-134° C. ¹H NMR (400 MHz, DMSO) δ 13.22 (s, 1H), 9.90 (s, 1H), 8.24 (d, J=1.3 Hz, 1H), 8.01 (d, J=7.6 Hz, 1H), 7.76 (m, 2H), 7.67 (t, J=7.4 Hz, 1H), 7.43 (t, J=7.0 Hz, 2H), 7.32 (s, 1H), 7.25 (t, J=7.1 Hz, 2H), 7.17 (d, J=9.1 Hz, 1H), 5.28 (s, 1H), 4.54 (s, 2H). ¹³C NMR (101 MHz, DMSO) δ 193.9, 167.3, 145.9, 144.9, 141.2, 138.4, 135.5, 133.0, 132.3, 130.5, 130.4, 130.3, 129.9, 128.8, 127.9, 126.6, 124.7, 123.8, 123.3, 117.0, 62.9. HRMS (ESI): calcd for C₂₁H₁₅N₂O₆, (M−H)⁻ 391.0936, found 391.0932. HPLC purity: 95.83%.

### 2-(4-((3-Chlorophenyl)amino)-3-nitrobenzoyl)benzoic Acid (C3-076)

The title compound was prepared from the hydrolysis of 20c (70 mg, 0.18 mmol) in 1N NaOH (0.9 ml) and THF (0.9 ml). Yellow solid, 45 mg, 63% yield, mp 159-161° C. ¹H NMR (400 MHz, DMSO) δ 13.24 (s, 1H), 9.85 (s, 1H), 8.24 (d, J=1.8 Hz, 1H), 8.01 (d, J=7.5 Hz, 1H), 7.78 (s, 1H), 7.74 (s, 1H), 7.67 (s, 1H), 7.54-7.45 (m, 2H), 7.42 (d, J=7.3 Hz, 1H), 7.40-7.30 (m, 2H), 7.23 (d, J=9.0 Hz, 1H). ¹³C NMR (101 MHz, DMSO) δ 193.9, 167.4, 144.9, 141.2, 140.5, 135.6, 134.2, 133.2, 132.9, 131.6, 130.6, 130.4, 130.3, 128.4, 127.7, 127.5, 126.2, 125.2, 123.7, 117.5. HRMS (ESI): calcd for C₂₀H₁₂ClN₂O₅, (M−H)⁻ 395.044, found 395.0434. HPLC purity: 96.87%.

### 2-(4-((3-(tert-Butyl)phenyl)amino)-3-nitrobenzoyl)benzoic Acid (C3-050)

The title compound was prepared from the hydrolysis of 21c (70 mg, 0.16 mmol) in 1N NaOH (0.8 ml) and THF (0.8 ml). Yellow solid, 56 mg, 84% yield, mp 110-112° C. ¹H NMR (400 MHz, DMSO) δ 13.22 (s, 1H), 9.91 (s, 1H), 8.24 (s, 1H), 8.01 (d, J=7.6 Hz, 1H), 7.79 (d, J=8.8 Hz, 1H), 7.77-7.70 (m, 1H), 7.67 (t, J=7.4 Hz, 1H), 7.46-7.31 (m, 4H), 7.19 (d, J=7.3 Hz, 1H), 7.15 (d, J=9.1 Hz, 1H), 1.30 (s, 9H). ¹³C NMR (101 MHz, DMSO) δ 193.9, 167.4, 153.0, 145.9, 141.3, 138.1, 135.5, 132.8, 132.2, 130.8, 130.4, 130.3, 129.7, 128.7, 127.8, 126.6, 123.7, 122.8, 122.6, 116.9, 35.0, 31.5. HRMS (ESI): calcd for C₂₄H₂₁N₂O₅, (M−H)⁻ 417.1456, found 417.1455. HPLC purity: 98.35%.

### 2-(4-((3-Carbamoylphenyl)amino)-3-nitrobenzoyl)benzoic Acid (C3-010)

The title compound was prepared from the hydrolysis of 22c (70 mg, 0.17 mmol) in 1N NaOH (0.8 ml) and THF (0.8 ml). Yellow solid, 43 mg, 62% yield, mp 139-141° C. ¹H NMR (400 MHz, DMSO) δ 13.21 (s, 1H), 9.95 (s, 1H), 8.25 (d, J=1.6 Hz, 1H), 8.02 (s, 1H), 8.01 (d, J=7.5 Hz, 2H), 7.86 (s, 1H), 7.80 (d, J=6.3 Hz, 2H), 7.75 (t, J=7.6 Hz, 1H), 7.67 (t, J=7.4 Hz, 1H), 7.60-7.50 (m, 2H), 7.49-7.39 (m, 2H), 7.17 (d, J=9.1 Hz, 1H). ¹³C NMR (101 MHz, DMSO) δ 193.9, 167.6, 167.3, 145.6, 141.2, 138.8, 136.2, 135.6, 133.0, 132.6, 130.5, 130.4, 130.1, 128.6, 128.4, 127.8, 126.9, 125.6, 124.6, 117.2. HRMS (ESI): calcd for C₂₁H₁₄N₃O₆, (M−H)⁻ 404.0888, found 404.0882. HPLC purity: 96.05%.

### 2-(4-((3,4-Dichlorophenyl)amino)-3-nitrobenzoyl)benzoic Acid (C3-064)

The title compound was prepared from the hydrolysis of 23c (45 mg, 0.1 mmol) in 1N NaOH (0.5 ml) and THF (0.5 ml). Yellow solid, 30 mg, 70% yield, mp 115-117° C. ¹H NMR (400 MHz, DMSO) δ 13.24 (s, 1H), 9.84 (s, 1H), 8.24 (s, 1H), 8.02 (d, J=7.3 Hz, 1H), 7.85-7.63 (m, 5H), 7.43 (d, J=7.3 Hz, 1H), 7.38 (d, J=7.5 Hz, 1H), 7.27 (d, J=9.0 Hz, 1H). ¹³C NMR (101 MHz, DMSO) δ 193.8, 167.3, 144.5, 141.1, 139.4, 135.6, 135.5, 133.8, 132.9, 132.8, 132.3, 131.7, 130.4, 128.2, 128.1, 127.9, 127.8, 126.8, 125.1, 117.8. HRMS (ESI): calcd for C₂₀H₁₁Cl₂N₂O₅, (M−H)⁻ 429.0051, found 429.0041. HPLC purity: 95.09%.

### 2-(4-((4-Methoxyphenyl)amino)-3-nitrobenzoyl)benzoic Acid (C3-049)

The title compound was prepared from the hydrolysis of 24c (61 mg, 0.15 mmol) in 1N NaOH (0.8 ml) and THF (0.8 ml). Yellow solid, 42 mg, 71% yield, mp 225-226° C. ¹H NMR (400 MHz, DMSO) δ 13.17 (s, 1H), 9.87 (s, 1H), 8.22 (s, 1H), 8.01 (d, J=7.6 Hz, 1H), 7.77 (d, J=9.8 Hz, 1H), 7.73 (d, J=7.2 Hz, 1H), 7.67 (t, J=7.4 Hz, 1H), 7.42 (d, J=7.3 Hz, 1H), 7.29 (d, J=8.7 Hz, 2H), 7.05 (d, J=8.7 Hz, 2H), 6.99 (d, J=9.1 Hz, 1H), 3.80 (s, 3H). ¹³C NMR (101 MHz, DMSO) δ 193.8, 167.3, 158.4, 147.0, 141.3, 135.4, 132.9, 131.5, 130.9, 130.4, 130.3, 128.9, 128.0, 127.8, 126.0, 116.7, 115.4, 55.8. HRMS (ESI): calcd for C₂₁H₁₅N₂O₆, (M−H)⁻ 391.0936, found 391.0929. HPLC purity: 98.67%.

### 2-(3-Nitro-4-(phenylamino)benzoyl)benzoic Acid (C3-011)

The title compound was prepared from the hydrolysis of 25c (70 mg, 0.18 mmol) in 1N NaOH (0.9 ml) and THF (0.9 ml). Yellow solid, 55 mg, 82% yield, mp 184-186° C. ¹H NMR (400 MHz, DMSO) δ 13.28 (s, 1H), 9.90 (s, 1H), 8.23 (d, J=1.7 Hz, 1H), 8.01 (d, J=7.6 Hz, 1H), 7.78 (dd, J=9.0, 1.5 Hz, 1H), 7.73 (t, J=7.4 Hz, 1H), 7.66 (t, J=7.3 Hz, 1H), 7.48 (t, J=7.7 Hz, 2H), 7.41 (d, J=7.4 Hz, 1H), 7.38 (d, J=7.6 Hz, 2H), 7.31 (t, J=7.3 Hz, 1H), 7.15 (d, J=9.0 Hz, 1H). ¹³C NMR (101 MHz, DMSO) δ 193.9, 167.5, 145.8, 141.3, 138.6, 135.5, 132.8, 132.3, 130.8, 130.4, 130.2, 130.2, 128.7, 127.7, 126.8, 126.8, 125.7, 116.9. HRMS (ESI): calcd for C₂₀H₁₃N₂O₅, (M−H)⁻ 361.083, found 361.0828. HPLC purity: 100.00%.

### 2-(3-Nitro-4-(quinolin-8-ylamino)benzoyl)benzoic Acid (C3-014)

The title compound was prepared from the hydrolysis of 26c (50 mg, 0.12 mmol) in 1N NaOH (0.6 ml) and THF (0.6 ml), deep yellow solid, 36 mg, yield 73%, mp 151-153° C. ¹H NMR (400 MHz, DMSO) δ 11.17 (s, 1H), 8.97 (s, 1H), 8.46 (d, J=7.4 Hz, 1H), 8.31 (s, 1H), 8.01 (d, J=4.5 Hz, 1H), 7.91 (d, J=6.6 Hz, 1H), 7.83 (s, 2H), 7.76 (d, J=7.5 Hz, 1H), 7.70-7.52 (m, 4H), 7.29 (d, J=4.2 Hz, 1H). ¹³C NMR (101 MHz, DMSO) δ 150.2, 142.5, 141.1, 140.3, 137.2, 135.8, 135.3, 134.3, 131.0, 130.2, 129.6, 129.4, 129.1, 128.0, 127.3, 126.9, 123.5, 123.06, 117.5, 117.2. HRMS (ESI): calcd for C₂₃H₁₄N₃O₅, (M−H)⁻ 412.0939, found 412.0937. HPLC purity: 96.02%.

### 2-(3-Nitro-4-(thiazol-2-ylamino)benzoyl)benzoic Acid (C3-034)

The title compound was prepared from the hydrolysis of 27c (60 mg, 0.16 mmol) in 1N NaOH (0.8 ml) and THF (0.8 ml). Yellow solid, 39 mg, 66% yield, mp 99-100° C. ¹H NMR (400 MHz, DMSO) δ 13.23 (s, 1H), 10.78 (s, 1H), 8.44 (d, J=8.4 Hz, 1H), 8.18 (s, 1H), 8.03 (d, J=7.6 Hz, 1H), 7.92 (d, J=8.8 Hz, 1H), 7.77 (t, J=7.3 Hz, 1H), 7.70 (t, J=7.3 Hz, 1H), 7.46 (m, 2H), 7.30 (s, 1H). ¹³C NMR (101 MHz, DMSO) δ 194.1, 167.3, 161.9, 141.0, 140.3, 139.2, 135.9, 135.2, 133.1, 130.5, 130.5, 130.3, 129.7, 127.9, 127.1, 120.4, 114.5. HRMS (ESI): calcd for C₁₇H₁₀N₃O₅S, (M−H)⁻ 368.0347, found 368.0343. HPLC purity: 98.63%.

### 2-(4-((2-Aminocyclohexyl)amino)-3-nitrobenzoyl)benzoic Acid (C3-007)

The title compound was prepared from the hydrolysis of 28c (50 mg, 0.13 mmol) in 1N NaOH (0.6 ml) and THF (0.6 ml). Yellow solid, 33 mg, 68% yield, decomposition temperature 265° C. ¹H NMR (400 MHz, DMSO) δ 8.15 (m, 2H), 7.94 (dd, J=5.5, 3.4 Hz, 1H), 7.62 (d, J=0.5 Hz, 1H), 7.47 (dd, J=5.4, 3.3 Hz, 2H), 7.17 (d, J=9.2 Hz, 1H), 7.12 (dd, J=5.3, 3.3 Hz, 1H), 3.94 (m, 1H), 3.20 (m, 1H), 1.96-1.80 (m, 2H), 1.65 (d, J=10.3 Hz, 2H), 1.55 (m, 1H), 1.43 (m, 1H), 1.38-1.10 (m, 2H). ¹³C NMR (101 MHz, DMSO) δ 195.5, 169.5, 147.0, 141.3, 138.7, 135.6, 131.1, 123.0, 129.6, 128.8, 128.2, 126.7, 126.2, 115.1, 55.2, 53.7, 31.6, 31.1, 24.4, 23.9HRMS (ESI): calcd for C₂₀H₂₀N₃O₅, (M−H)⁻ 382.1408, found 382.1406. HPLC purity: 98.62%.

### 2-(4-(Benzylamino)-3-nitrobenzoyl)benzoic Acid (C3-035)

The title compound was prepared from the hydrolysis of 29c (50 mg, 0.13 mmol) in 1N NaOH (0.6 ml) and THF (0.6 ml). Yellow solid, 35 mg, 73% yield, mp 88-90° C. ¹H NMR (400 MHz, DMSO) δ 13.18 (s, 1H), 9.18 (t, J=5.8 Hz, 1H), 8.22 (d, J=1.7 Hz, 1H), 7.99 (d, J=7.5 Hz, 1H), 7.71 (t, J=7.1 Hz, 2H), 7.65 (t, J=7.4 Hz, 1H), 7.42-7.32 (m, 4H), 7.27 (t, J=6.7 Hz, 1H), 7.03 (d, J=9.2 Hz, 1H), 4.71 (d, J=6.1 Hz, 2H). ¹³C NMR (101 MHz, DMSO) δ 193.9, 167.4, 147.7, 141.37, 138.3, 135.6, 132.8, 131.1, 130.4, 130.4, 130.2, 129.1, 128.9, 127.8, 127.7, 127.4, 125.0, 115.7, 46.3. HRMS (ESI): calcd for C₂₁H₁₅N₂O₅, (M−H)⁻ 375.0986, found 375.0982. HPLC purity: 99.55%.

### 2-(4-((2-Fluorobenzyl)amino)-3-nitrobenzoyl)benzoic Acid (C3-036)

The title compound was prepared from the hydrolysis of 30c (50 mg, 0.13 mmol) in 1N NaOH (0.6 ml) and THF (0.6 ml). Yellow solid, 35 mg, 73% yield, mp 100-102° C. ¹H NMR (400 MHz, DMSO) δ 13.18 (s, 1H), 9.06 (t, J=5.9 Hz, 1H), 8.24 (d, J=1.9 Hz, 1H), 7.99 (d, J=7.5 Hz, 1H), 7.73 (dd, J=18.3, 8.4 Hz, 2H), 7.65 (t, J=7.2 Hz, 1H), 7.36 (m, 3H), 7.28-7.21 (m, 1H), 7.17 (t, J=7.4 Hz, 1H), 7.03 (d, J=9.1 Hz, 1H), 4.77 (d, J=6.1 Hz, 2H). ¹³C NMR (101 MHz, DMSO) δ 193.9, 167.4, 160.6 (d, J=245.4 Hz), 147.6, 141.3, 135.8, 132.8, 131.2, 130.3 (d, J=17.2 Hz), 129.8 (d, J=8.1 Hz), 129.3 (d, J=4.2 Hz), 128.9, 127.8, 125.2, 125.1, 125.1, 125.1, 124.9, 115.9 (d, J=21.2 Hz), 115.4, 40.5 (d, J=4.0 Hz). HRMS (ESI): calcd for C₂₁H₁₄FN₂O₅, (M−H)⁻ 393.0892, found 393.0890. HPLC purity: 99.58%.

### 2-(3-Nitro-4-(piperidin-1-yl)benzoyl)benzoic Acid (C3-013)

The title compound was prepared from the hydrolysis of 31c (70 mg, 0.19 mmol) in 1N NaOH (1 ml) and THF (1 ml). Yellow solid, 58 mg, 86% yield, mp 191-193° C. ¹H NMR (400 MHz, DMSO) δ 13.23 (s, 1H), 8.00 (d, J=7.6 Hz, 1H), 7.93 (d, J=2.0 Hz, 1H), 7.72 (t, J=7.0 Hz, 1H), 7.70-7.67 (m, 1H), 7.65 (t, J=5.5 Hz, 1H), 7.41 (d, J=7.3 Hz, 1H), 7.31 (d, J=8.9 Hz, 1H), 3.14 (s, 4H), 1.60 (s, 6H). ¹³C NMR (101 MHz, DMSO) δ 193.9, 167.4, 148.9, 141.4, 138.8, 134.0, 132.8, 130.5, 130.3, 130.3, 128.0, 127.8, 127.5, 120.4, 51.7, 25.6, 23.7. HRMS (ESI): calcd for C₁₉H₁₇N₂O₅, (M−H)⁻ 353.1143, found 353.1140. HPLC purity: 99.21%.

### 2-(4-Morpholino-3-nitrobenzoyl)benzoic Acid (C3-012)

The title compound was prepared from the hydrolysis of 32c (60 mg, 0.17 mmol) in 1N NaOH (0.8 ml) and THF (0.8 ml), yellow solid, 42 mg, 69% yield, mp 192-193° C. ¹H NMR (400 MHz, DMSO) δ 13.23 (s, 1H), 8.01 (d, J=7.6 Hz, 1H), 7.98 (d, J=1.8 Hz, 1H), 7.78-7.70 (m, 2H), 7.67 (t, J=7.5 Hz, 1H), 7.43 (d, J=7.3 Hz, 1H), 7.35 (d, J=8.9 Hz, 1H), 3.70 (m, 4H), 3.17 (m, 4H). ¹³C NMR (101 MHz, DMSO) δ 194.1, 167.3, 148.3, 141.2, 139.4, 134.2, 133.3, 130.4, 130.2, 128.5, 127.8, 120.6, 66.2, 50.9. HRMS (ESI): calcd for C₁₈H₁₅N₂O₆, (M−H)⁻ 355.0936, found 355.0933. HPLC purity: 100.00%.

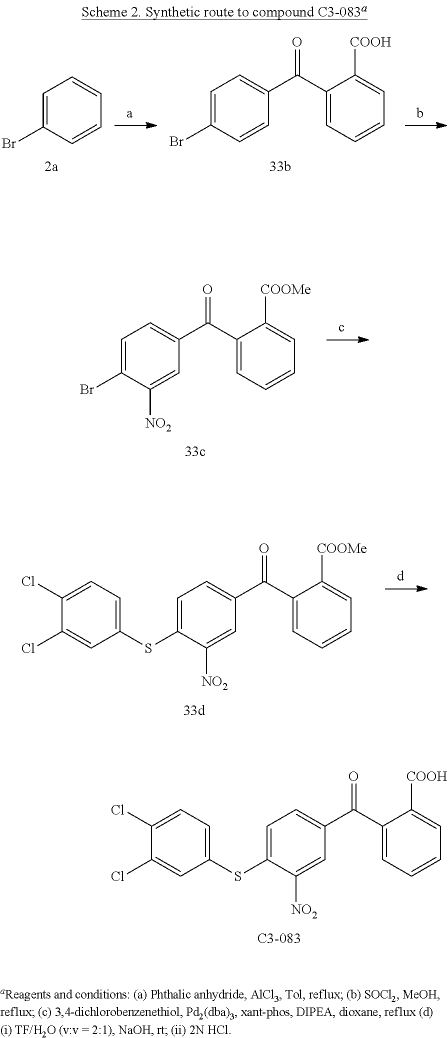 [SMILES: Brc1ccccc1 ; O=C(O)c1ccccc1C(=O)c1ccc(Br)cc1 ; COC(=O)c1ccccc1C(=O)c1ccc(Br)c([N+](=O)[O-])c1 ; COC(=O)c1ccccc1C(=O)c1ccc(Sc2ccc(Cl)c(Cl)c2)c([N+](=O)[O-])c1 ; O=C(O)c1ccccc1C(=O)c1ccc(Sc2ccc(Cl)c(Cl)c2)c([N+](=O)[O-])c1]

### 2-(4-Bromobenzoyl)benzoic Acid (33b)

To a suspension of the phthalic anhydride (2 g, 13.5 mmol) in bromobenzene (11.38 ml, 108 mmol) was added aluminum chloride. The mixture was heated to 90° C. for 2 h and then cooled to room temperature. Ice was added follow by conc. HCl (5 ml) and the mixture was extracted with DCM and then washed with 10% Na₂CO₃ solution. The Na₂CO₃ washings were combined and acidified to PH3 with conc. HCl. The resulting precipitate was collected by filtration and dried in vacuum. 33b was obtained as white solid without further purification, 3.5 g, 85% yield. ¹H NMR (400 MHz, DMSO) δ 13.25 (s, 1H), 8.00 (d, J=7.5 Hz, 1H), 7.79-7.60 (m, 4H), 7.54 (d, J=8.4 Hz, 2H), 7.45 (d, J=7.5 Hz, 1H).

### Methyl 2-(4-bromobenzoyl)benzoate (33c)

Compound 33b (500 mg) was dissolved in MeOH (10 ml). SOCl₂ (10 drops) was added and then hit to reflux overnight. The reaction mixture was cooled down to r.t. and white solid precipitate was collected and dried to provide the 33c as white solid, 450 mg. ¹H NMR (400 MHz, CDCl₃) δ 8.06 (d, J=7.7 Hz, 1H), 7.69-7.63 (m, 1H), 7.63-7.55 (m, 5H), 7.39 (d, J=7.6 Hz, 1H), 3.67 (s, 3H).

### Methyl 2-(4-((3,4-dichlorophenyl)thio)benzoyl)benzoate (33d)

To a round-bottom-flask were added compound 33c (64 mg, 02 mmol) i-Pr₂NEt (66 μL, 0.4 mmol), dry 1,4-dioxane. The mixture was evacuated and backfilled with nitrogen (3 cycles). Pd₂(dba)₃ (18 mg, 0.02 mmol), xant-phos (23 mg, 0.04 mmol) and the thiol were added and then the mixture was degassed twice more. The mixture was then allowed to heat to reflux overnight. The reaction mixture was then allowed to reach to ambient temperature. The reaction mixture was then filtered and concentrated. The crude product was purified by flash column chromatography on silica gel to afford the desired thioether 33d as yellow solid, 65 mg, 77.9% yield. ¹H NMR (400 MHz, CDCl₃) δ 8.07 (d, J=7.5 Hz, 1H), 7.69 (d, J=8.4 Hz, 2H), 7.65 (dd, J=7.5, 1.0 Hz, 1H), 7.59 (td, J=7.6, 0.9 Hz, 1H), 7.55 (d, J=2.1 Hz, 1H), 7.46 (d, J=8.3 Hz, 1H), 7.43-7.38 (m, 1H), 7.30 (d, J=2.1 Hz, 1H), 7.26 (d, J=8.5 Hz, 2H), 3.69 (s, 3H).

### 2-(4-((3,4-Dichlorophenyl)thio)benzoyl)benzoic Acid (C3-083)

The hydrolysis of 33d (42 mg, 0.1 mmol) in 1N NaOH (0.5 ml) and dioxane (0.5 ml) at r.t. provide compound C3-083 as grey solid, 20 mg, 50% yield. mp 164-166° C. ¹H NMR (400 MHz, DMSO) δ 13.22 (s, 1H), 7.99 (d, J=7.6 Hz, 1H), 7.72 (dd, J=12.2, 7.3 Hz, 3H), 7.66 (t, J=7.5 Hz, 1H), 7.59 (d, J=8.2 Hz, 2H), 7.42 (d, J=6.7 Hz, 2H), 7.37 (d, J=8.3 Hz, 2H). ¹³C NMR (101 MHz, DMSO) δ 196.0, 167.3, 141.6, 135.9, 134.2, 133.6, 133.0, 132.7, 132.3, 132.0, 130.3, 129.2, 127.8. HRMS (ESI): calcd for C₂₀H₁₁Cl₂O₃S, (M−H)⁻ 400.9811, found 400.9806. HPLC purity 95.07%.

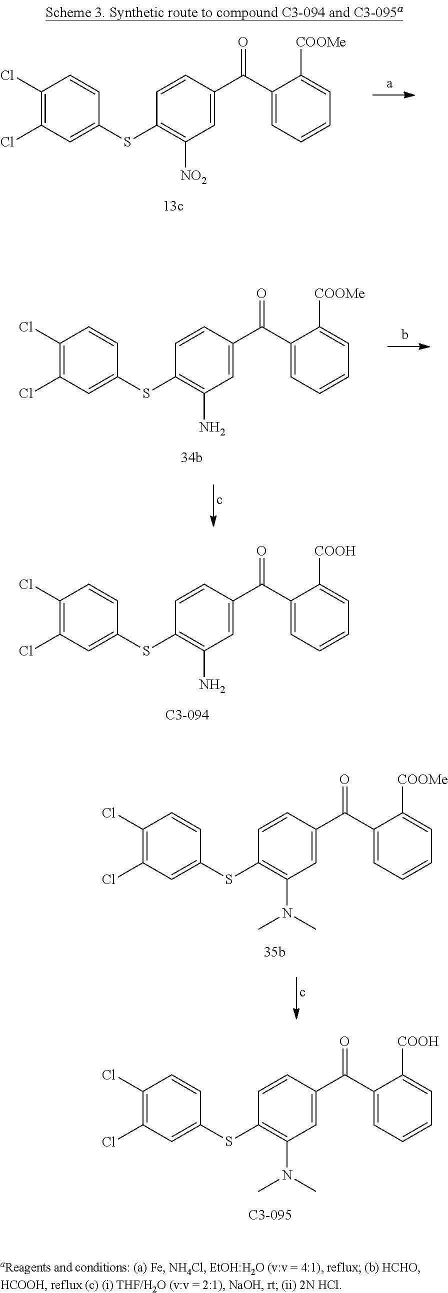 [SMILES: COC(=O)c1ccccc1C(=O)c1ccc(Sc2ccc(Cl)c(Cl)c2)c([N+](=O)[O-])c1 ; COC(=O)c1ccccc1C(=O)c1ccc(Sc2ccc(Cl)c(Cl)c2)c(N)c1 ; Nc1cc(C(=O)c2ccccc2C(=O)O)ccc1Sc1ccc(Cl)c(Cl)c1 ; COC(=O)c1ccccc1C(=O)c1ccc(Sc2ccc(Cl)c(Cl)c2)c(N(C)C)c1 ; CN(C)c1cc(C(=O)c2ccccc2C(=O)O)ccc1Sc1ccc(Cl)c(Cl)c1]

### Methyl 2-(3-amino-4-((3,4-dichlorophenyl)thio)benzoyl)benzoate (34b)

13c (46 mg, 0.1 mmol), 28 mg (0.5 mmol) of iron, and saturated ammonium chloride solution (1 ml) and EtOH (4 ml) were added to a round-bottom flask and heated to reflux for 1 h. after which, the reaction mixture was cooled down to r.t. and filtered. The precipitate was washed with ethyl acetate and the combined solution was then washed twice with NaCl solution. The organic phase was dried with Na₂SO₄ to provide 34b (35 mg) as yellow jelly which was used without further purification. ¹H NMR (400 MHz, CDCl₃) δ 8.06 (d, J=7.7 Hz, 1H), 7.67 (t, J=7.3 Hz, 1H), 7.60 (t, J=7.5 Hz, 1H), 7.45 (dd, J=7.5, 4.0 Hz, 2H), 7.31 (d, J=8.4 Hz, 1H), 7.24 (d, J=1.5 Hz, 1H), 7.17 (d, J=2.1 Hz, 1H), 7.02 (dd, J=8.0, 1.6 Hz, 1H), 6.95 (dd, J=8.4, 2.1 Hz, 1H), 4.40 (s, 2H), 3.70 (s, 3H).

### 2-(3-Amino-4-((3,4-dichlorophenyl)thio)benzoyl)benzoic Acid (C3-094)

34b (35 mg) was hydrolyzed with 1N NaOH (0.5 ml) and dioxane (0.5 ml) at r.t. provide compound C3-094 as yellow solid, 26 mg, 62% yield. mp 188-190° C. ¹H NMR (400 MHz, DMSO) δ 13.17 (s, 1H), 7.98 (d, J=7.5 Hz, 1H), 7.71 (t, J=7.2 Hz, 1H), 7.64 (t, J=7.3 Hz, 1H), 7.55 (d, J=8.5 Hz, 1H), 7.40 (d, J=7.9 Hz, 2H), 7.32 (d, J=1.9 Hz, 1H), 7.12 (d, J=1.1 Hz, 1H), 7.02 (dd, J=8.5, 2.0 Hz, 1H), 6.80 (dd, J=7.9, 1.3 Hz, 1H), 5.73 (s, 2H). ¹³C NMR (101 MHz, DMSO) δ 167.5, 150.5, 142.3, 140.0, 137.3, 137.0, 132.7, 132.1, 131.5, 130.8, 130.1, 128.9, 128.7, 127.6, 117.0, 116.1, 115.6. HRMS (ESI): calcd for C₂₀H₁₂Cl₂NO₃S, (M−H)⁻ 415.992, found 415.9923. HPLC purity 95.38%.

### Methyl 2-(4-((3,4-dichlorophenyl)thio)-3-(dimethylamino)benzoyl)benzoate (35b)

Compound 34b (130 mg, 0.3 mmol), 37% HCHO solution (2 ml) and HCOOH (2 ml) were added to a round-bottom flask and heated to 100° C. overnight. The reaction mixture was cooled down to r.t. and NaHCO₃ solution was added. The mixture was extracted with EA, the organic phase was washed by NaCl solution, and purified by chromatography to provide 35b as yellow solid, 40 mg, 29% yield. ¹H NMR (400 MHz, CDCl₃) δ 8.02 (d, J=7.7 Hz, 1H), 7.72 (d, J=1.5 Hz, 1H), 7.58 (dqd, J=22.0, 7.4, 0.9 Hz, 3H), 7.45 (d, J=8.3 Hz, 1H), 7.36 (d, J=7.1 Hz, 1H), 7.31 (dd, J=8.3, 1.9 Hz, 1H), 7.05 (dd, J=8.2, 1.6 Hz, 1H), 6.73 (d, J=8.2 Hz, 1H), 3.67 (s, 3H), 2.81 (s, 6H).

### 2-(4-((3,4-Dichlorophenyl)thio)-3-(dimethylamino)benzoyl)benzoic Acid (C3-095)

35b (40 mg, 0.09 mmol) was hydrolyzed with 1N NaOH (0.5 ml) dioxane (0.5 ml) at r.t. to provide compound C3-095 as grey solid, 20 mg, 52% yield. mp 144-146° C. ¹H NMR (400 MHz, DMSO) δ 7.95 (d, J=6.4 Hz, 1H), 7.76 (s, 1H), 7.69 (dd, J=13.7, 7.5 Hz, 2H), 7.63 (d, J=6.7 Hz, 1H), 7.53 (s, 1H), 7.40 (dd, J=27.2, 6.7 Hz, 2H), 7.02 (d, J=7.4 Hz, 1H), 6.80 (d, J=7.5 Hz, 1H), 2.71 (s, 6H). ¹³C NMR (101 MHz, DMSO) δ 167.5, 151.2, 141.8, 138.6, 136.1, 135.4, 134.3, 133.1, 132.7, 132.6, 132.4, 132.3, 130.9, 130.1, 130.0, 128.2, 127.7, 125.8, 119.3, 44.2. HRMS (ESI): calcd for C₂₂H₁₆Cl₂NO₃S, (M−H)⁻ 444.0233, found 444.0225. HPLC purity: 90.27%.

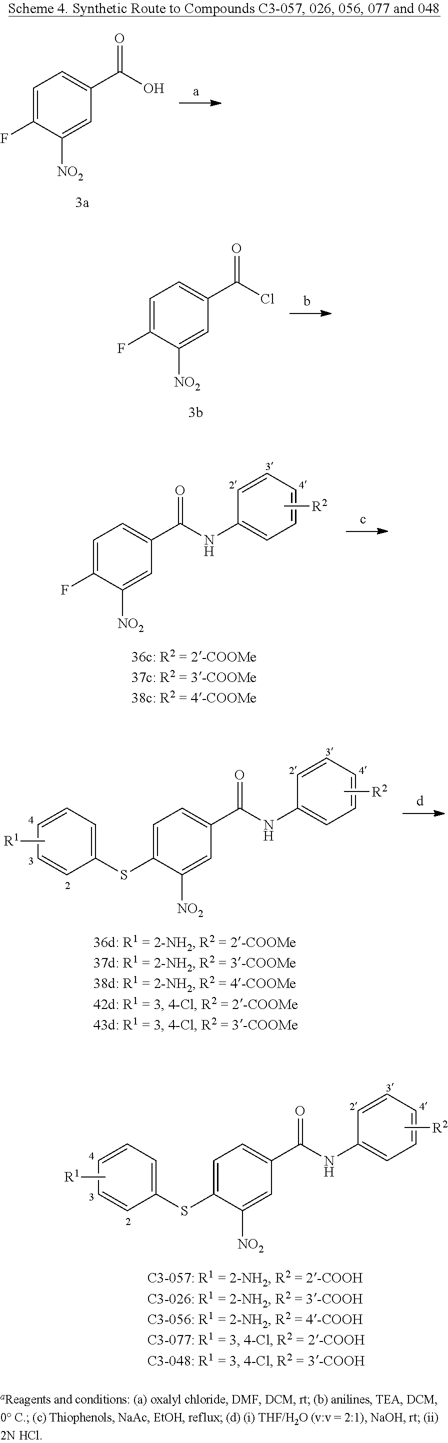

### Methyl 2-(4-fluoro-3-nitrobenzamido)benzoate (36c)

To a solution of 4-fluoro-3-nitrobenzoic acid 3a (185 mg, 1 mmol) in dry CH₂C12 (5 ml) was added oxalyl chloride (2 mmol) and 5 drops of dry DMF under nitrogen atmosphere. The solution was stirred at room temperature overnight. The solution was evaporated to dryness to remove the excess of oxalyl chloride. The resulting acyl chloride solution was then added dropwise to a solution of methyl 2-aminobenzoate (1 mmol) and Et₃N (0.279 ml, 2 mmol) in dry CH₂Cl₂ (5 ml) under nitrogen atmosphere and the mixture was stirred at room temperature overnight. The solution was then quenched with water. The aqueous phase was extracted with CH₂Cl₂ and the organic phase was combined together, washed with 1 M HCl and saturated NaCl aqueous solution successively, dried over anhydrous Na₂SO₄, filtered, and concentrated under reduced pressure. The crude material was purified via silica gel column to provide 36c; yellow solid, 190 mg, 60%, yield. ¹H NMR (400 MHz, CDCl₃) δ 12.30 (s, 1H), 8.88 (d, J=8.4 Hz, 1H), 8.81 (d, J=6.6 Hz, 1H), 8.34 (d, J=5.6 Hz, 1H), 8.14 (d, J=7.8 Hz, 1H), 7.67 (t, J=7.6 Hz, 1H), 7.49 (t, J=9.2 Hz, 1H), 7.21 (t, J=7.5 Hz, 1H), 4.02 (s, 3H).

### Methyl 3-(4-fluoro-3-nitrobenzamido)benzoate (37c)

The title compound was prepared by condensing Methyl 3-Aminobenzoate and 4-Fluoro-3-nitrobenzoic Acid in a similar manner as described for compound 36c; yellow solid, 200 mg, 63% yield. ¹H NMR (400 MHz, CDCl₃) δ 8.63 (dd, J=6.8, 2.1 Hz, 1H), 8.30-8.24 (m, 1H), 8.19 (s, 1H), 8.07 (s, 1H), 8.03 (d, J=8.2 Hz, 1H), 7.90 (d, J=7.7 Hz, 1H), 7.55-7.44 (m, 2H), 3.95 (s, 3H).

### Synthesis of Methyl 4-(4-fluoro-3-nitrobenzamido)benzoate (38c)

The title compound was prepared by condensing Methyl 4-Aminobenzoate and 4-Fluoro-3-nitrobenzoic Acid in a similar manner as described for compound 36c; yellow solid, 130 mg, 41% yield. ¹H NMR (400 MHz, CDCl₃) δ 8.61 (d, J=4.9 Hz, 1H), 8.32-8.22 (m, 1H), 8.12 (d, J=8.6 Hz, 2H), 7.77 (d, J=8.6 Hz, 2H), 7.49 (t, J=9.3 Hz, 1H), 3.95 (s, 3H).

### Methyl 3-(4-((2-aminophenyl)thio)-3-nitrobenzamido)benzoate (36d)

To a flask were added 36c (64 mg, 0.2 mmol), 2-Aminobenzenethiol (30 mg, 0.24 mmol), NaAc (82 mg, 1 mmol) and EtOH 5 ml. The mixture was stirred at room temperature overnight. Water was added and then the aqueous was extracted with ethyl acetate. The combined organic layers were dried over Na₂SO₄ and purified by chromatography to provide 36d as pale-yellow solid, 45 mg, 53% yield. ¹H NMR (400 MHz, CDCl₃) δ 8.27 (s, 2H), 7.94 (d, J=8.0 Hz, 1H), 7.45 (d, J=7.5 Hz, 1H), 7.38 (d, J=8.4 Hz, 1H), 7.36-7.29 (m, 2H), 6.83 (td, J=8.0, 1.8 Hz, 3H), 6.65 (t, J=7.5 Hz, 1H), 6.53 (d, J=8.2 Hz, 1H), 4.49 (d, J=5.7 Hz, 2H), 4.30 (s, 2H), 3.89 (s, 3H).

### Methyl 3-(4-((2-aminophenyl)thio)-3-nitrobenzamido)benzoate (37d)

To a flask were added 37c (64 mg, 0.2 mmol), 2-Aminobenzenethiol (30 mg, 0.24 mmol), NaAc (82 mg, 1 mmol) and EtOH 5 ml. The mixture was stirred at room temperature overnight. Water was added and then the aqueous was extracted with ethyl acetate. The combined organic layers were dried over Na₂SO₄ and purified by chromatography to provide 37d as yellow solid, 75 mg, 89% yield. ¹H NMR (400 MHz, CDCl₃) δ 9.57 (s, 1H), 8.86 (s, 1H), 8.13 (s, 1H), 8.06 (d, J=8.0 Hz, 1H), 7.95 (d, J=8.6 Hz, 1H), 7.79 (d, J=7.6 Hz, 1H), 7.43 (t, J=7.9 Hz, 2H), 7.34 (t, J=7.7 Hz, 1H), 6.95 (d, J=8.5 Hz, 1H), 6.84 (m, 2H), 4.39 (s, 2H), 3.91 (s, 4H).

### Methyl 4-(4-((2-aminophenyl)thio)-3-nitrobenzamido)benzoate (38d)

To a flask were added 38c (64 mg, 0.2 mmol), 2-Aminobenzenethiol (30 mg, 0.24 mmol), NaAc (82 mg, 1 mmol) and EtOH 5 ml. The mixture was stirred at room temperature overnight. Water was added and then the aqueous was extracted with ethyl acetate. The combined organic layers were dried over Na₂SO₄ and purified by chromatography to provide 38d as yellow solid, 60 mg, 71% yield. ¹H NMR (400 MHz, DMSO) δ 10.75 (s, 1H), 8.86 (d, J=1.5 Hz, 1H), 8.09 (dd, J=8.5, 1.6 Hz, 1H), 7.95 (dd, J=22.2, 8.8 Hz, 4H), 7.39 (d, J=7.6 Hz, 1H), 7.30 (t, J=7.3 Hz, 1H), 6.91 (d, J=8.6 Hz, 1H), 6.86 (d, J=8.1 Hz, 1H), 6.68 (t, J=7.2 Hz, 1H), 5.62 (s, 2H), 3.84 (s, 3H).

### Methyl 2-(4-((3,4-dichlorophenyl)thio)-3-nitrobenzamido)benzoate (42d)

To a flask were added 36c (64 mg, 0.2 mmol), 3, 4-Dichlorobenzenethiol (43 mg, 0.24 mmol), NaAc (82 mg, 1 mmol) and EtOH 5 ml. The mixture was stirred at room temperature overnight. Water was added and then the aqueous was extracted with ethyl acetate. The combined organic layers were dried over Na₂SO₄ and purified by chromatography to provide 42d as yellow solid, 83 mg, 87% yield. ¹H NMR (400 MHz, CDCl₃) δ 12.24 (s, 1H), 8.98 (d, J=1.6 Hz, 1H), 8.89 (d, J=8.5 Hz, 1H), 8.13 (d, J=7.9 Hz, 1H), 8.04 (dd, J=8.5, 1.8 Hz, 1H), 7.76 (d, J=1.8 Hz, 1H), 7.65 (t, J=8.6 Hz, 2H), 7.48 (dd, J=8.2, 1.8 Hz, 1H), 7.19 (t, J=7.6 Hz, 1H), 7.04 (d, J=8.5 Hz, 1H), 4.00 (s, 3H).

### Methyl 3-(4-((3,4-dichlorophenyl)thio)-3-nitrobenzamido)benzoate (43d)

To a flask were added 37c (64 mg, 0.2 mmol), 3, 4-Dichlorobenzenethiol (43 mg, 0.24 mmol), NaAc (82 mg, 1 mmol) and EtOH 5 ml. The mixture was stirred at room temperature overnight. Water was added and then the aqueous was extracted with ethyl acetate. The combined organic layers were dried over Na₂SO₄ and purified by chromatography to provide 43d as yellow solid, 72 mg, 75% yield. ¹H NMR (400 MHz, CDCl₃) δ 8.76 (d, J=1.9 Hz, 1H), 8.19 (t, J=2.0 Hz, 1H), 8.03 (m, 2H), 7.98 (dd, J=8.5, 2.0 Hz, 1H), 7.88 (d, J=7.8 Hz, 1H), 7.74 (d, J=2.0 Hz, 1H), 7.64 (d, J=8.3 Hz, 1H), 7.53-7.45 (m, 2H), 7.03 (d, J=8.5 Hz, 1H), 3.95 (s, 3H).

General Procedure for the Synthesis of Compound Target Compounds

The methyl esters of the title compounds (36d˜38d, 42d and 43d) were hydrolyzed with 1 M NaOH in THF (1:1) at room temperature overnight. The mixture was then diluted with a small amount of water and washed twice with CH₂Cl₂. The aqueous solution was acidified by the addition of 2 M HCl. The precipitate was collected by filtration and washed with water to afford the title compounds. If the compound was not pure at this stage of the procedure, it was purified by silica gel column.

### 2-(4-((2-Aminophenyl)thio)-3-nitrobenzamido)benzoic Acid (C3-057)

The title compound was prepared from the hydrolysis of 36d (42 mg, 0.1 mmol) in 1N NaOH (0.5 ml) and THF (0.5 ml). Yellow solid, 30 mg, 73% yield, mp 259-261° C. ¹H NMR (400 MHz, DMSO) δ 13.86 (s, 1H), 12.27 (s, 1H), 8.81 (s, 1H), 8.61 (d, J=8.3 Hz, 1H), 8.06 (d, J=7.2 Hz, 2H), 7.68 (t, J=7.5 Hz, 1H), 7.39 (d, J=7.4 Hz, 1H), 7.30 (t, J=7.4 Hz, 1H), 7.24 (t, J=7.5 Hz, 1H), 6.96 (d, J=8.5 Hz, 1H), 6.85 (d, J=8.1 Hz, 1H), 6.68 (t, J=7.3 Hz, 1H), 5.65 (s, 2H). ¹³C NMR (101 MHz, DMSO) δ 170.5, 162.7, 151.3, 145.3, 142.3, 141.0, 137.7, 134.7, 132.9, 132.2, 131.7, 131.7, 128.0, 124.9, 123.8, 120.6, 117.6, 117.3, 115.7, 109.2. HRMS (ESI): calcd for C₂₀H₁₄N₃O₅S, (M−H)⁻ 408.0660, found 408.0656. HPLC purity: 99.30%.

### 3-(4-((2-Aminophenyl)thio)-3-nitrobenzamido)benzoic Acid (C3-026)

The title compound was prepared from the hydrolysis of 37d (42 mg, 0.1 mmol) in 1N NaOH (0.5 ml) and THF (0.5 ml). Yellow solid, 35 mg, 85% yield, mp 251-252° C. ¹H NMR (400 MHz, DMSO) δ 13.02 (s, 1H), 10.64 (s, 1H), 8.89 (d, J=1.7 Hz, 1H), 8.39 (s, 1H), 8.10 (dd, J=8.5, 1.7 Hz, 1H), 8.05 (d, J=7.9 Hz, 1H), 7.70 (d, J=7.7 Hz, 1H), 7.50 (t, J=7.9 Hz, 1H), 7.39 (dd, J=7.6, 1.0 Hz, 1H), 7.33-7.26 (m, 1H), 6.88 (dd, J=19.5, 8.3 Hz, 2H), 6.68 (t, J=7.1 Hz, 1H), 5.63 (s, 2H). ¹³C NMR (101 MHz, DMSO) δ 167.7, 163.6, 151.3, 145.2, 141.6, 139.4, 137.7, 133.1, 132.8, 131.8, 129.4, 127.4, 125.7, 125.2, 124.8, 121.7, 117.3, 115.7, 109.4. HRMS (ESI): calcd for C₂₀H₁₄N₃O₅S, (M−H)⁻ 408.0660, found 408.0653. HPLC purity: 98.48%.

### 4-(4-((2-Aminophenyl)thio)-3-nitrobenzamido)benzoic Acid (C3-056)

The title compound was prepared from the hydrolysis of 38d (42 mg, 0.1 mmol) in 1N NaOH (0.5 ml) and THF (0.5 ml). Yellow solid, 33 mg, 82.5% yield, mp 247-249° C. ¹H NMR (400 MHz, DMSO) δ 12.79 (s, 1H), 10.73 (s, 1H), 8.86 (s, 1H), 8.09 (d, J=7.7 Hz, 1H), 7.95 (m, 2H), 7.90 (m, 2H), 7.39 (m, 1H), 7.30 (m, 1H), 6.90 (m, 1H), 6.85 (m, 1H), 6.67 (m, 1H), 5.63 (s, 2H). ¹³C NMR (101 MHz, DMSO) δ 167.4, 163.9, 151.3, 145.2, 143.3, 141.8, 137.7, 133.1, 132.8, 131.8, 130.7, 127.4, 126.3, 125.8, 120.1, 117.3, 115.7, 109.4. HRMS (ESI): calcd for C₂₀H₁₄N₃O₅S, (M−H)⁻ 408.0660, found 408.0652. HPLC purity: 95.33%.

### 2-(4-((3,4-Dichlorophenyl)thio)-3-nitrobenzamido)benzoic Acid (C3-077)

The title compound was prepared from the hydrolysis of 42d (48 mg, 0.1 mmol) in 1N NaOH (0.5 ml) and THF (0.5 ml). Yellow solid, 27 mg, 58.3% yield, mp 279-281° C. ¹H NMR (400 MHz, DMSO) δ 12.78 (s, 1H), 8.79 (s, 1H), 8.61 (d, J=8.3 Hz, 1H), 8.11 (d, J=8.5 Hz, 1H), 8.06 (d, J=7.8 Hz, 1H), 8.03 (d, J=1.8 Hz, 1H), 7.85 (d, J=8.3 Hz, 1H), 7.71-7.59 (m, 2H), 7.21 (dd, J=14.5, 8.0 Hz, 2H). ¹³C NMR (101 MHz, DMSO) δ 170.5, 162.3, 145.1, 141.4, 140.9, 137.2, 135.9, 134.3, 134.1, 134.0, 133.3, 132.8, 132.8, 132.7, 131.7, 131.1, 129.8, 124.8, 123.7, 120.4. HRMS (ESI): calcd for C₂₀H₁₁Cl₂N₂O₅S, (M−H)⁻ 460.9771, found 460.9767. HPLC purity: 95.02%.

### 3-(4-((3,4-Dichlorophenyl)thio)-3-nitrobenzamido)benzoic Acid (C3-048)

The title compound was prepared from the hydrolysis of 43d (48 mg, 0.1 mmol) in 1N NaOH (0.5 ml) and THF (0.5 ml). Yellow solid, 40 mg, 86.3% yield, mp 280-282° C. ¹H NMR (400 MHz, DMSO) δ 10.70 (s, 1H), 8.89 (s, 1H), 8.38 (s, 1H), 8.15 (d, J=8.1 Hz, 1H), 8.04 (d, J=7.6 Hz, 1H), 8.00 (s, 1H), 7.83 (d, J=8.1 Hz, 1H), 7.70 (d, J=7.3 Hz, 1H), 7.65 (d, J=8.0 Hz, 1H), 7.49 (t, J=7.6 Hz, 1H), 7.12 (d, J=8.4 Hz, 1H). ¹³C NMR (101 MHz, DMSO) δ 167.6, 163.4, 145.0, 140.9, 139.4, 137.1, 135.9, 134.2, 133.8, 133.3, 132.8, 132.7, 131.9, 131.3, 129.4, 129.3, 125.4, 125.3, 124.9, 121.7. HRMS (ESI): calcd for C₂₀H₁₁Cl₂N₂O₅S, (M−H)⁻ 460.9771, found 460.9766. HPLC purity: 96.76%.

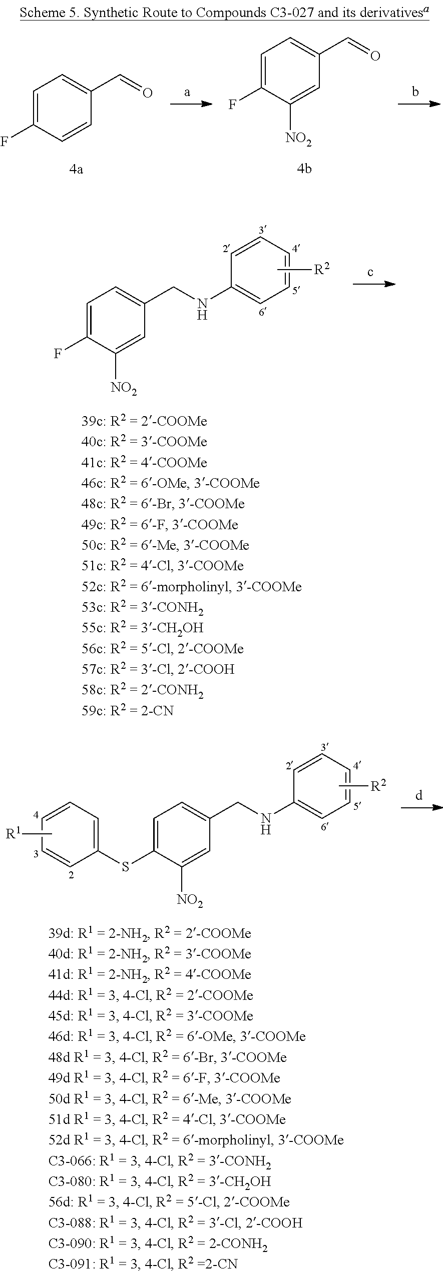

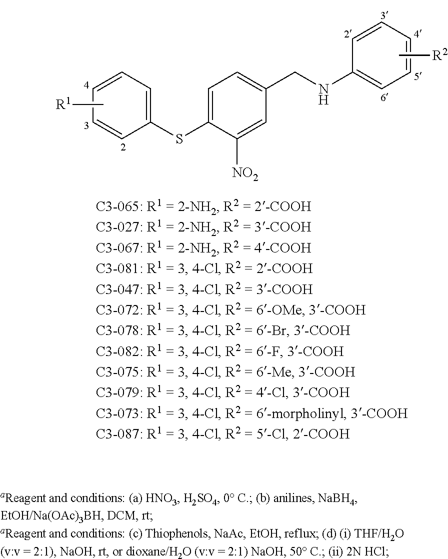

### 4-Fluoro-3-nitrobenzaldehyde (4b)

4-Fluorobenzaldehyde (4a, 1.24 g, 10 mmol) was slowly added dropwise to a solution of H₂SO₄ (8 mL) and HNO₃ (1 mL) at −5° C. The temperature was kept under 5° C. Then the solution was warmed to room temperature over 1 h. After pouring into ice, the precipitate was collected by filtration and washed by ice-water, dried in vacuum to provide compound 4b; white solid, 1.55 g, yield 92%. ¹H NMR (400 MHz, CDCl₃) δ 10.06 (s, 1H), 8.61 (dd, J=7.0, 1.6 Hz, 1H), 8.22 (ddd, J=8.3, 4.0, 1.9 Hz, 1H), 7.57-7.45 (m, 1H).

### Methyl 2-((4-fluoro-3-nitrobenzyl)amino)benzoate (39c)

In a dried round bottom flask, 4b (169 mg, 1 mmol) and Methyl 2-Aminobenzoate (151 mg, 1 mmol) were dissolved in CH₂Cl₂ (10 ml) and stirred at room temperature for 30 min. Then the reaction was cooled down to 0° C. and NaBH(OAc)₃ (636 mg, 3 mmol) was added in three portions over 1 h. The reaction was allowed to stir for 0° C. to room temperature overnight. The reaction was quenched with a saturated aqueous solution of sodium bicarbonate and then extracted with CH₂Cl₂. The combined organic layers were dried with Na₂SO₄, filtered and the solvent was removed under reduced pressure. The crude was purified by column chromatography to give the title compound; yellow solid, 155 mg, yield 51%. ¹H NMR (400 MHz, CDCl₃) δ 8.33 (s, 1H), 8.05 (d, J=6.8 Hz, 1H), 7.97 (d, J=7.9 Hz, 1H), 7.64 (m, 1H), 7.35-7.23 (m, 2H), 6.68 (t, J=7.4 Hz, 1H), 6.51 (d, J=6.9 Hz, 1H), 4.53 (d, J=5.0 Hz, 2H), 3.91 (s, 3H).

### Methyl 3-((4-fluoro-3-nitrobenzyl)amino)benzoate (40c)

The title compound was prepared from 4b (169 mg, 1 mmol) and Methyl 3-Aminobenzoate (151 mg, 1 mmol) in a similar manner as described for compound 40c; yellow solid, 220 mg, 72% yield. ¹H NMR (400 MHz, CDCl₃) δ 8.06 (dd, J=7.0, 2.0 Hz, 1H), 7.64 (m, 1H), 7.42 (d, J=7.7 Hz, 1H), 7.28-7.25 (m, 2H), 7.22 (d, J=7.8 Hz, 1H), 6.76 (dd, J=8.1, 2.3 Hz, 1H), 4.45 (d, J=5.5 Hz, 2H), 3.88 (s, 3H).

### Methyl 4-((4-fluoro-3-nitrobenzyl)amino)benzoate (41c)

The title compound was prepared from 4b (169 mg, 1 mmol) and Methyl 3-Aminobenzoate (151 mg, 1 mmol) in a similar manner as described for compound 36c; yellow solid, 125 mg, yield 41%. ¹H NMR (400 MHz, CDCl₃) δ 8.06 (d, J=6.8 Hz, 1H), 7.88 (d, J=8.5 Hz, 2H), 7.67-7.60 (m, 1H), 7.30-7.25 (m, 1H), 6.58 (d, J=8.6 Hz, 2H), 4.70 (s, 1H), 4.49 (d, J=5.7 Hz, 2H), 3.87 (s, 3H).

### Methyl 3-((4-fluoro-3-nitrobenzyl)amino)-4-methoxybenzoate (46c)

In a dried round bottom flask, 4-Fluoro-3-nitrobenzaldehyde (4b, 85 mg, 0.5 mmol), Methyl 3-amino-4-methoxybenzoate (91 mg, 0.5 mmol) and EtOH (5 ml) were stirred at room temperature for 1 h. the precipitate was collected by filtration and then dried in vacuum. The dried solid was then dissolved in CH₂Cl₂ (3 ml). NaBH(OAc)₃ (636 mg, 3 mmol) was added in three portions over 1 h and stir at room temperature overnight. The reaction was quenched with a saturated aqueous solution of NH₄Cl and then extracted with CH₂Cl₂. The combined organic layers were dried with Na₂SO₄, filtered and the solvent was removed under reduced pressure. The crude was purified by column chromatography to give the title compound; yellow solid, 60 mg, yield 36%. ¹H NMR (400 MHz, CDCl₃) δ 8.09 (dd, J=7.0, 1.8 Hz, 1H), 7.72-7.63 (m, 1H), 7.49 (dd, J=8.4, 1.9 Hz, 1H), 7.33-7.24 (m, 1H), 7.15 (d, J=1.8 Hz, 1H), 6.83 (d, J=8.4 Hz, 1H), 4.82 (t, J=5.6 Hz, 1H), 4.48 (d, J=5.8 Hz, 2H), 3.95 (s, 3H), 3.85 (s, 3H).

### Methyl 4-bromo-3-((4-fluoro-3-nitrobenzyl)amino)benzoate (48c)

In a dried round bottom flask, 4-Fluoro-3-nitrobenzaldehyde 4b, (85 mg, 0.5 mmol), Methyl 3-amino-4-bromobenzoate (115 mg, 0.5 mmol) and EtOH (5 ml) were stirred at room temperature for 1 h. Then the reaction was cooled down to 0° C. and NaBH₄ (32 mg, 1 mmol) was added in two portions over 30 min. The reaction was allowed to stir for 0° C. to room temperature overnight (or until complete by TLC). The reaction was quenched with a saturated aqueous solution of NH₄Cl and then extracted with CH₂Cl₂. The combined organic layers were dried with Na₂SO₄, filtered and the solvent was removed under reduced pressure. The crude was purified by column chromatography to give the title compound; yellow solid, 100 mg, 52% yield. ¹H NMR (400 MHz, CDCl₃) δ 8.08 (d, J=6.3 Hz, 1H), 7.65 (s, 1H), 7.54 (d, J=7.9 Hz, 1H), 7.30 (t, J=7.7 Hz, 2H), 7.18 (s, 1H), 4.99 (s, 1H), 4.54 (d, J=5.0 Hz, 2H), 3.86 (s, 3H).

### Methyl 4-fluoro-3-((4-fluoro-3-nitrobenzyl)amino)benzoate (49c)

The title compound was prepared in an analogous fashion as described in section for compound 48c using 4-Fluoro-3-nitrobenzaldehyde (4b, 85 mg, 0.5 mmol) and Methyl 3-amino-4-fluorobenzoate (85 mg, 0.5 mmol) through reductive amination reaction; yellow solid, 97 mg, 60% yield. ¹H NMR (400 MHz, CDCl₃) δ 8.11 (d, J=6.4 Hz, 1H), 7.67 (d, J=7.7 Hz, 1H), 7.49-7.39 (m, 1H), 7.33 (d, J=9.8 Hz, 1H), 7.28-7.24 (m, 1H), 7.13-7.02 (m, 1H), 4.52 (s, 3H), 3.88 (s, 3H).

### Methyl 3-((4-fluoro-3-nitrobenzyl)amino)-4-methylbenzoate (50c)

The title compound was prepared in an analogous fashion as described in section for compound 48c using 4-Fluoro-3-nitrobenzaldehyde (4b, 85 mg, 0.5 mmol) and Methyl 3-amino-4-methylbenzoate (84 mg, 0.5 mmol) through reductive amination reaction; yellow solid, 73 mg, 46% yield. ¹H NMR (400 MHz, CDCl₃) δ 8.09 (d, J=6.9 Hz, 1H), 7.71-7.64 (m, 1H), 7.41 (d, J=7.6 Hz, 1H), 7.32-7.26 (m, 1H), 7.17 (s, 2H), 4.52 (d, J=4.4 Hz, 2H), 3.86 (s, 3H), 2.27 (s, 3H).

### Methyl 2-chloro-5-((4-fluoro-3-nitrobenzyl)amino)benzoate (51c)

The title compound was prepared in an analogous fashion as described in section for compound 48c using 4-Fluoro-3-nitrobenzaldehyde (4b, 85 mg, 0.5 mmol) and Methyl 5-amino-2-chlorobenzoate (93 mg, 0.5 mmol) through reductive amination reaction; yellow solid, 150 mg, 88% yield. ¹H NMR (400 MHz, CDCl₃) δ 8.07 (d, J=6.6 Hz, 1H), 7.68-7.59 (m, 1H), 7.31 (d, J=10.2 Hz, 1H), 7.24 (d, J=8.7 Hz, 1H), 7.05 (d, J=2.6 Hz, 1H), 6.64 (dd, J=8.6, 2.7 Hz, 1H), 4.44 (d, J=5.3 Hz, 2H), 4.37 (s, 1H), 3.92 (s, 3H).

### Methyl 3-((4-fluoro-3-nitrobenzyl)amino)-4-morpholinobenzoate (52c)

The title compound was prepared in an analogous fashion as described in section for compound 48c using 4-Fluoro-3-nitrobenzaldehyde (4b, 85 mg, 0.5 mmol) and Methyl 3-Amino-4-morpholinobenzoate (118 mg, 0.5 mmol) through reductive amination reaction; yellow solid, 88 mg, 45% yield. ¹H NMR (400 MHz, CDCl₃) δ 8.07 (d, J=7.1 Hz, 1H), 7.69-7.59 (m, 1H), 7.49 (dd, J=8.1, 1.6 Hz, 1H), 7.31 (d, J=10.4 Hz, 1H), 7.18 (d, J=1.4 Hz, 1H), 7.08 (d, J=8.1 Hz, 1H), 5.19 (s, 1H), 4.78 (s, 1H), 4.49 (d, J=5.6 Hz, 2H), 3.93-3.86 (m, 4H), 3.86 (s, 3H), 3.04-2.95 (m, 4H).

### 3-((4-fluoro-3-nitrobenzyl)amino)benzamide (53c)

The title compound was prepared in an analogous fashion as described in section for compound 39c using 4-Fluoro-3-nitrobenzaldehyde (4b, 85 mg, 0.5 mmol) and 3-Aminobenzamide (68 mg, 0.5 mmol) through reductive amination reaction; yellow solid, 65 mg, 45% yield. ¹H NMR (400 MHz, DMSO) δ 8.13 (d, J=6.7 Hz, 1H), 7.76 (s, 2H), 7.63-7.48 (m, 1H), 7.17 (s, 1H), 7.14-6.96 (m, 3H), 6.71 (d, J=7.3 Hz, 1H), 6.57 (s, 1H), 4.40 (s, 2H).

### (3-((4-Fluoro-3-nitrobenzyl)amino)phenyl)methanol (55c)

The title compound was prepared in an analogous fashion as described in section for compound 39c using 4-Fluoro-3-nitrobenzaldehyde (4b, 85 mg, 0.5 mmol) and (3-Aminophenyl)methanol (62 mg, 0.5 mmol) through reductive amination reaction; yellow solid, 88 mg, 64% yield. ¹H NMR (400 MHz, CDCl₃) δ 8.08 (d, J=6.7 Hz, 1H), 7.71-7.61 (m, 1H), 7.28 (t, J=9.5 Hz, 1H), 7.18 (t, J=7.8 Hz, 1H), 6.76 (d, J=7.4 Hz, 1H), 6.65 (s, 1H), 6.52 (d, J=7.9 Hz, 1H), 4.62 (s, 2H), 4.44 (s, 2H), 4.29 (s, 1H), 1.62 (s, 1H).

### 4-Chloro-2-((4-fluoro-3-nitrobenzyl)amino)benzoic acid (56c)

In a dried round bottom flask, 4-Fluoro-3-nitrobenzaldehyde (4b, 85 mg, 0.5 mmol), Methyl 2-amino-4-chlorobenzoate (93 mg, 0.5 mmol) and EtOH (1 ml) were stirred at room for 24 h. Solvent was removed under reduced pressure. The residue was dissolved in CH₂Cl₂ (3 ml). NaBH(OAc)₃ (316 mg, 1.5 mmol) was added in three portions over 1 h and the mixture was stirred at room temperature overnight. The reaction was quenched with a saturated aqueous solution of NH₄Cl and then extracted with CH₂Cl₂. The combined organic layers were dried with Na₂SO₄, filtered and the solvent was removed under reduced pressure. The crude was purified by column chromatography to give the title compound; yellow solid, white solid, 30 mg, yield 18%. ¹H NMR (400 MHz, CDCl₃) δ 8.40 (s, 1H), 8.05 (dd, J=6.8, 1.8 Hz, 1H), 7.90 (d, J=8.5 Hz, 1H), 7.68-7.59 (m, 1H), 7.35-7.29 (m, 1H), 6.65 (dd, J=8.6, 1.8 Hz, 1H), 6.51 (d, J=1.6 Hz, 1H), 4.51 (d, J=5.8 Hz, 2H), 3.90 (s, 3H).

### 2-Chloro-6-((4-fluoro-3-nitrobenzyl)amino)benzoic acid (57c)

In a dried round bottom flask, 4-Fluoro-3-nitrobenzaldehyde (4b, 85 mg, 0.5 mmol), Methyl 2-amino-4-chlorobenzoate (93 mg, 0.5 mmol) and EtOH (5 ml) were stirred at room for 24 h. NaBH(OAc)₃ (316 mg, 1.5 mmol) was added in three portions over 1 h and the mixture was stirred at room temperature for 2 days. The reaction was quenched with an excess amount of saturated aqueous solution of NaHCO₃. The aqueous phase was washed twice with CH₂Cl₂. The aqueous layer was acidified, and the precipitate was collected by filtration, dried in vacuum to provide the title compound as gray solid, 120 mg, yield 74%. ¹H NMR (400 MHz, CDCl₃) δ 8.09-8.00 (m, 1H), 7.62 (dd, J=4.6, 3.4 Hz, 1H), 7.31 (d, J=10.4 Hz, 1H), 7.17 (t, J=8.2 Hz, 1H), 6.82 (d, J=8.0 Hz, 1H), 6.43 (d, J=8.5 Hz, 1H), 4.51 (s, 2H).

### 2-((4-Fluoro-3-nitrobenzyl)amino)benzamide (58c)

The title compound was prepared in an analogous fashion as described in section for compound 48c using 4-Fluoro-3-nitrobenzaldehyde (4b, 85 mg, 0.5 mmol) and 2-Aminobenzamide (68 mg, 0.5 mmol) through reductive amination reaction; yellow solid, 100 mg, 69% yield. ¹H NMR (400 MHz, CDCl₃) δ 8.52 (s, 1H), 8.05 (d, J=6.8 Hz, 1H), 7.69-7.61 (m, 1H), 7.45 (d, J=7.8 Hz, 1H), 7.27 (t, J=8.9 Hz, 1H), 6.68 (t, J=7.5 Hz, 1H), 6.51 (d, J=8.4 Hz, 1H), 5.73 (m, 2H), 4.51 (d, J=5.8 Hz, 2H).

### 2-((4-Fluoro-3-nitrobenzyl)amino)benzonitrile (59c)

The title compound was prepared in an analogous fashion as described in section for compound 48c using 4-Fluoro-3-nitrobenzaldehyde (4b, 85 mg, 0.5 mmol) and 2-Aminobenzonitrile (59 mg, 0.5 mmol) through reductive amination reaction; yellow solid, 92 mg, 68% yield. ¹H NMR (400 MHz, CDCl₃) δ 8.52 (t, J=4.7 Hz, 1H), 8.06 (dd, J=6.9, 1.8 Hz, 1H), 7.70-7.58 (m, 1H), 7.45 (d, J=7.8 Hz, 1H), 7.33-7.23 (m, 1H), 6.68 (t, J=7.5 Hz, 1H), 6.51 (d, J=8.4 Hz, 1H), 5.77 (s, 2H), 4.51 (d, J=5.9 Hz, 2H).

### Methyl 2-((4-((2-aminophenyl)thio)-3-nitrobenzyl)amino)benzoate (39d)

To a flask was added Methyl 2-((4-fluoro-3-nitrobenzyl)amino)benzoate (39c, 61 mg, 0.2 mmol), 2-Aminobenzenethiol (30 mg, 0.24 mmol), NaAc (82 mg, 1 mmol) and EtOH 2 ml. The mixture was heated to reflux for 4 h. After cooling to rt., the precipitate was collected via filtration and washed with appropriate EtOH and water successively and dried in vacuum to give the titled compounds. Otherwise, water was added and then the aqueous was extracted by EA. The combined organic layers were dried over Na₂SO₄ and purification by chromatography to provide the title compounds 36d as yellow solid, 70 mg, 85% yield. H NMR (400 MHz, CDCl₃) δ 8.27 (s, 2H), 7.95 (d, J=7.9 Hz, 1H), 7.45 (d, J=7.5 Hz, 1H), 7.38 (d, J=8.4 Hz, 1H), 7.36-7.29 (m, 2H), 6.85-6.82 (m, J=9.6, 5.9 Hz, 3H), 6.65 (t, J=7.5 Hz, 1H), 6.53 (d, J=8.4 Hz, 1H), 4.48 (d, J=5.7 Hz, 2H), 4.30 (s, 2H), 3.89 (s, 3H).

### Methyl 3-((4-((2-aminophenyl)thio)-3-nitrobenzyl)amino)benzoate (40d)

The title compound was prepared from Methyl 3-((4-fluoro-3-nitrobenzyl)amino)benzoate (40c, 64 mg, 0.2 mg) and 2-Aminobenzenethiol (30 mg, 0.24 mmol) in a similar manner as described for compound 39d; yellow solid, 66 mg, 80% yield. ¹H NMR (400 MHz, CDCl₃) δ 8.29 (s, 1H), 7.46 (d, J=7.6 Hz, 1H), 7.40 (t, J=8.2 Hz, 2H), 7.34 (t, J=7.7 Hz, 1H), 7.28 (s, 1H), 7.23 (t, J=7.9 Hz, 1H), 6.89-6.79 (m, 3H), 6.76 (dd, J=8.0, 2.1 Hz, 1H), 4.42 (d, J=5.3 Hz, 2H), 4.31 (s, 2H), 3.89 (s, 3H).

### Methyl 4-((4-((2-aminophenyl)thio)-3-nitrobenzyl)amino)benzoate (41d)

The title compound was prepared from Methyl 4-((4-fluoro-3-nitrobenzyl)amino)benzoate (41c, 61 mg, 0.2 mmol) and 2-Aminobenzenethiol (30 mg, 0.24 mmol) in a similar manner as described for compound 39d; yellow solid, 66 mg, 80% yield. ¹H NMR (400 MHz, CDCl₃) δ 8.27 (s, 1H), 7.86 (d, J=8.5 Hz, 2H), 7.46 (d, J=7.6 Hz, 1H), 7.35 (m, 2H), 6.84 (m, 3H), 6.56 (d, J=8.6 Hz, 2H), 4.60 (t, J=5.1 Hz, 1H), 4.44 (d, J=5.6 Hz, 2H), 4.30 (s, 2H), 3.86 (s, 3H).

### Methyl 2-((4-((3,4-dichlorophenyl)thio)-3-nitrobenzyl)amino)benzoate (44d)

The title compound was prepared from Methyl 2-((4-fluoro-3-nitrobenzyl)amino)benzoate (39c, 64 mg, 0.2 mg) and 3,4-Dichlorobenzenethiol (43 mg, 0.24 mmol) in a similar manner as described for compound 39d; yellow solid, 72 mg, 78% yield. ¹H NMR (400 MHz, CDCl₃) δ 8.29 (t, J=5.5 Hz, 1H), 8.23 (s, 1H), 7.96 (dd, J=8.0, 1.4 Hz, 1H), 7.69 (d, J=2.0 Hz, 1H), 7.56 (d, J=8.3 Hz, 1H), 7.46-7.38 (m, 2H), 7.35-7.29 (m, 1H), 6.87 (d, J=8.4 Hz, 1H), 6.67 (t, J=7.5 Hz, 1H), 6.52 (d, J=8.4 Hz, 1H), 4.51 (d, J=5.9 Hz, 2H), 3.90 (s, 3H).

### Methyl 3-((4-((3,4-dichlorophenyl)thio)-3-nitrobenzyl)amino)benzoate (45d)

The title compound was prepared from Methyl 3-((4-fluoro-3-nitrobenzyl)amino)benzoate (40c, 64 mg, 0.2 mg) and 3,4-Dichlorobenzenethiol (43 mg, 0.24 mmol) in a similar manner as described for compound 39d; yellow solid, 76 mg, 82% yield. ¹H NMR (400 MHz, CDCl₃) δ 8.25 (s, 1H), 7.68 (d, J=1.9 Hz, 1H), 7.56 (d, J=8.3 Hz, 1H), 7.49-7.35 (m, 4H), 7.23 (t, J=7.9 Hz, 1H), 6.87 (d, J=8.3 Hz, 1H), 6.76 (dd, J=8.0, 2.1 Hz, 1H), 4.44 (d, J=5.7 Hz, 2H), 4.38 (d, J=5.6 Hz, 1H), 3.89 (s, 3H).

### Methyl 3-((4-((3,4-dichlorophenyl)thio)-3-nitrobenzyl)amino)-4-methoxybenzoate (46d)

The title compound was prepared from Methyl 3-((4-fluoro-3-nitrobenzyl)amino)-4-methoxybenzoate (46c, 67 mg, 0.2 mmol) and 3,4-Dichlorobenzenethiol (43 mg, 0.24 mmol) in a similar manner as described for compound 39d; yellow solid, 66 mg, 67% yield. ¹H NMR (400 MHz, DMSO) δ 8.21 (s, 1H), 7.90 (s, 1H), 7.77 (dd, J=8.3, 2.4 Hz, 1H), 7.57 (t, J=8.7 Hz, 2H), 7.26 (d, J=8.3 Hz, 1H), 7.00 (d, J=8.3 Hz, 1H), 6.92 (d, J=12.0 Hz, 2H), 6.09 (s, 1H), 4.43 (d, J=4.4 Hz, 2H), 3.89 (d, J=2.1 Hz, 3H), 3.72 (d, J=2.2 Hz, 3H).

### Methyl 4-bromo-3-((4-((3,4-dichlorophenyl)thio)-3-nitrobenzyl)amino)benzoate (48d)

The title compound was prepared from Methyl 4-chloro-3-((4-fluoro-3-nitrobenzyl)amino)benzoate (48c, 77 mg, 0.2 mmol) and 3,4-Dichlorobenzenethiol (43 mg, 0.24 mmol) in a similar manner as described for compound 39d; yellow solid, 97 mg, 89% yield. ¹H NMR (400 MHz, CDCl₃) δ 8.27 (s, 1H), 7.71 (d, J=1.9 Hz, 1H), 7.57 (d, J=8.3 Hz, 1H), 7.54 (d, J=8.2 Hz, 1H), 7.43 (d, J=8.2 Hz, 2H), 7.31 (d, J=1.7 Hz, 1H), 7.19 (d, J=1.5 Hz, 1H), 6.89 (d, J=8.4 Hz, 1H), 4.93 (t, J=5.6 Hz, 1H), 4.53 (d, J=5.9 Hz, 2H), 3.88 (s, 3H).

### Methyl 3-((4-((3,4-dichlorophenyl)thio)-3-nitrobenzyl)amino)-4-fluorobenzoate (49d)

The title compound was prepared from Methyl 4-chloro-3-((4-fluoro-3-nitrobenzyl)amino)benzoate (49c, 100 mg, 0.31 mmol) and 3,4-Dichlorobenzenethiol (66.7 mg, 0.37 mmol) in a similar manner as described for compound 39d; yellow solid, 82 mg, 55% yield. ¹H NMR (400 MHz, CDCl₃) δ 8.27 (s, 1H), 7.70 (s, 1H), 7.57 (dd, J=8.2, 1.8 Hz, 1H), 7.43 (m, 3H), 7.31-7.25 (m, 1H), 7.12-6.99 (m, 1H), 6.90 (dd, J=8.3, 1.6 Hz, 1H), 4.49 (s, 3H), 3.88 (s, 3H).

### Methyl 3-((4-((3,4-dichlorophenyl)thio)-3-nitrobenzyl)amino)-4-methylbenzoate (50d)

The title compound was prepared from Methyl 4-chloro-3-((4-fluoro-3-nitrobenzyl)amino)benzoate (50c, 63 mg, 0.2 mmol) and 3,4-Dichlorobenzenethiol (43 mg, 0.24 mmol) in a similar manner as described for compound 39d; yellow solid, 58 mg, 61% yield. ¹H NMR (400 MHz, CDCl₃) δ 8.27 (s, 1H), 7.70 (d, J=1.7 Hz, 1H), 7.57 (d, J=8.3 Hz, 1H), 7.43 (m, 3H), 7.20-7.13 (m, 2H), 6.89 (d, J=8.4 Hz, 1H), 4.51 (d, J=5.7 Hz, 2H), 4.05 (t, J=5.4 Hz, 1H), 3.87 (s, 3H), 2.25 (s, 3H).

### Methyl 2-chloro-5-((4-((3,4-dichlorophenyl)thio)-3-nitrobenzyl)amino)benzoate (51d)

The title compound was prepared from Methyl 4-chloro-3-((4-fluoro-3-nitrobenzyl)amino)benzoate (51c, 67 mg, 0.2 mmol) and 3,4-Dichlorobenzenethiol (43 mg, 0.24 mmol) in a similar manner as described for compound 39d; yellow solid, 73 mg, 73% yield.

### Methyl 3-((4-((3,4-dichlorophenyl)thio)-3-nitrobenzyl)amino)-4-morpholinobenzoate (52d)

The title compound was prepared from Methyl 3-((4-fluoro-3-nitrobenzyl)amino)-4-morpholinobenzoate (52c, 78 mg, 0.2 mmol) and 3,4-Dichlorobenzenethiol (43 mg, 0.24 mmol) in a similar manner as described for compound 39d; yellow solid, 49 mg, 45% yield. ¹H NMR (400 MHz, CDCl₃) δ 8.25 (s, 1H), 7.71 (s, 1H), 7.57 (d, J=8.3 Hz, 1H), 7.48 (d, J=8.2 Hz, 1H), 7.42 (t, J=8.4 Hz, 2H), 7.19 (s, 1H), 7.07 (d, J=8.1 Hz, 1H), 6.89 (d, J=8.4 Hz, 1H), 5.16 (t, J=5.6 Hz, 1H), 4.47 (d, J=5.7 Hz, 2H), 3.88 (m, 7H), 3.10-2.91 (m, 4H).

### Methyl 4-chloro-2-((4-((3,4-dichlorophenyl)thio)-3-nitrobenzyl)amino)benzoate (56d)

The title compound was prepared from Methyl 4-chloro-2-((4-fluoro-3-nitrobenzyl)amino)benzoate (56c, 30 mg, 0.09 mmol) and 3,4-Dichlorobenzenethiol (18 mg, 0.1 mmol) in a similar manner as described for compound 39d; yellow solid, 23 mg, 44% yield. ¹H NMR (400 MHz, CDCl₃) δ 8.36 (t, J=5.4 Hz, 1H), 8.23 (s, 1H), 7.88 (d, J=8.5 Hz, 1H), 7.71 (d, J=1.9 Hz, 1H), 7.57 (d, J=8.3 Hz, 1H), 7.43 (dd, J=8.4, 2.0 Hz, 2H), 6.89 (d, J=8.4 Hz, 1H), 6.64 (dd, J=8.5, 1.8 Hz, 1H), 6.51 (d, J=1.5 Hz, 1H), 4.93 (d, J=4.8 Hz, 1H), 4.48 (d, J=5.8 Hz, 2H), 3.89 (s, 3H).

General Procedure for the Synthesis of Compound Target Compounds

The methyl esters of the intermediates (39d˜41d, 44d˜46d, 48d˜53d and 56d˜59d) were hydrolyzed with 1 M NaOH in dioxane (1:1) at 50° C. overnight. The mixture was then diluted with a small amount of water and washed twice with CH₂Cl₂. The aqueous solution was acidified by the addition of 2 M HCl. The precipitate was collected by filtration and washed with water to afford the title compounds.

### 2-((4-((2-Aminophenyl)thio)-3-nitrobenzyl)amino)benzoic Acid (C3-065)

The title compound was prepared from the hydrolysis of Methyl 2-((4-((2-aminophenyl)thio)-3-nitrobenzyl)amino)benzoate (39d) (41 mg, 0.1 mmol) in 1 M NaOH (0.3 ml) and dioxane (1 ml) at 50° C. overnight. Yellow solid, 21 mg, 53% yield, mp 201-203° C. ¹H NMR (400 MHz, DMSO) δ 12.77 (s, 1H), 8.38 (s, 1H), 8.22 (s, 1H), 7.80 (d, J=7.5 Hz, 1H), 7.54 (d, J=8.1 Hz, 1H), 7.33 (d, J=7.4 Hz, 1H), 7.27 (dd, J=16.9, 8.2 Hz, 2H), 6.82 (d, J=8.0 Hz, 1H), 6.74 (d, J=8.3 Hz, 1H), 6.63 (t, J=7.5 Hz, 2H), 6.57 (t, J=7.4 Hz, 1H), 5.53 (s, 2H), 4.52 (s, 2H). ¹³C NMR (101 MHz, DMSO) δ 170.4, 151.4, 150.7, 145.4, 138.3, 137.9, 135.9, 134.8, 133.4, 132.6, 132.2, 127.7, 124.6, 117.2, 115.6, 115.3, 112.1, 111.3, 110.1, 44.8. HRMS (ESI): calcd for C₂₀H₁₆N₃O₄S, (M−H)⁻ 394.0867, found 394.0862. HPLC purity: 99.32%.

### 3-((4-((2-Aminophenyl)thio)-3-nitrobenzyl)amino)benzoic Acid (C3-027)

The title compound was prepared from the hydrolysis of Methyl 3-((4-((2-aminophenyl)thio)-3-nitrobenzyl)amino)benzoate (40d) (41 mg, 0.1 mmol) in 1 M NaOH (0.3 ml) and dioxane (1 ml) at 50° C. overnight. Yellow solid, 27 mg, 68% yield, mp 105-107° C. ¹H NMR (400 MHz, DMSO) δ 12.66 (s, 1H), 8.25 (s, 1H), 7.55 (d, J=8.3 Hz, 1H), 7.33 (d, J=7.5 Hz, 1H), 7.26 (t, J=7.6 Hz, 1H), 7.15 (m, 3H), 6.82 (d, J=8.0 Hz, 1H), 6.75 (m, 2H), 6.63 (m, 2H), 5.53 (s, 2H), 4.34 (d, J=5.2 Hz, 2H). ¹³C NMR (101 MHz, DMSO) δ 168.3, 151.4, 148.7, 145.4, 138.7, 137.9, 135.8, 133.5, 132.6, 132.1, 129.5, 127.3, 124.6, 117.6, 117.2, 116.7, 115.6, 113.6, 110.1, 45.4. HRMS (ESI): calcd for C₂₀H₁₆N₃O₄S, (M−H)⁻ 394.0867, found 394.0863. HPLC purity: 97.18%.

### 4-((4-((2-aminophenyl)thio)-3-nitrobenzyl)amino)benzoic Acid (C3-067)

The title compound was prepared from the hydrolysis of Methyl 4-((4-((2-aminophenyl)thio)-3-nitrobenzyl)amino)benzoate (41d) (41 mg, 0.1 mmol) in 1 M NaOH (0.3 ml) and dioxane (1 ml) at 50° C. overnight. Yellow solid, 33 mg, 84% yield, decomposition temperature 250° C. ¹H NMR (400 MHz, DMSO) δ 12.04 (s, 1H), 8.23 (s, 1H), 7.64 (d, J=8.6 Hz, 2H), 7.54 (d, J=8.2 Hz, 1H), 7.33 (d, J=7.3 Hz, 1H), 7.26 (t, J=7.6 Hz, 1H), 7.13 (t, J=5.9 Hz, 1H), 6.82 (d, J=8.0 Hz, 1H), 6.73 (d, J=8.4 Hz, 1H), 6.64 (t, J=7.3 Hz, 1H), 6.59 (d, J=8.6 Hz, 2H), 5.52 (s, 2H), 4.38 (d, J=5.8 Hz, 2H). ¹³C NMR (101 MHz, DMSO) δ 152.2, 151.4, 145.4, 138.3, 137.9, 135.8, 133.6, 132.6, 131.6, 127.4, 124.7, 117.2, 115.6, 111.7, 110.1, 45.0. HRMS (ESI): calcd for C₂₀H₁₆N₃O₄S, (M−H)⁻ 394.0867, found 394.0863. HPLC purity: 96.24%.

### 2-((4-((3,4-Dichlorophenyl)thio)-3-nitrobenzyl)amino)benzoic Acid (C3-081)

The title compound was prepared from the hydrolysis of Methyl 2-((4-((3,4-dichlorophenyl)thio)-3-nitrobenzyl)amino)benzoate (44d) (46 mg, 0.1 mmol) in 1 M NaOH (0.3 ml) and dioxane (1 ml) at 50° C. overnight. Yellow solid, 22 mg, 49% yield, mp 171-173° C. ¹H NMR (400 MHz, DMSO) δ 9.56 (s, 1H), 8.17 (s, 1H), 7.91 (s, 1H), 7.84 (d, J=7.3 Hz, 1H), 7.78 (d, J=8.3 Hz, 1H), 7.56 (t, J=5.7 Hz, 2H), 7.06 (t, J=7.1 Hz, 1H), 7.00 (d, J=8.1 Hz, 1H), 6.50-6.34 (m, 2H), 4.46 (s, 2H). ¹³C NMR (101 MHz, DMSO) δ 150.1, 146.0, 140.8, 136.5, 135.3, 133.8, 133.6, 133.5, 133.0, 132.6, 132.4, 132.0, 129.9, 124.2, 118.6, 114.7, 110.9, 45.2. HRMS (ESI): calcd for C₂₀H₁₃Cl₂N₂O₄S, (M−H)⁻ 446.9979, found 446.9968. HPLC purity: 96.26%.

### 3-((4-((3,4-Dichlorophenyl)thio)-3-nitrobenzyl)amino)benzoic Acid (C3-047)

The title compound was prepared from the hydrolysis of Methyl 3-((4-((3,4-dichlorophenyl)thio)-3-nitrobenzyl)amino)benzoate (45d) (46 mg, 0.1 mmol) in 1 M NaOH (0.3 ml) and dioxane (1 ml) at 50° C. overnight. Yellow solid, 25 mg, 56% yield, mp 224-226° C. ¹H NMR (400 MHz, DMSO) δ 8.23 (s, 1H), 7.92 (d, J=1.9 Hz, 1H), 7.78 (d, J=8.3 Hz, 1H), 7.60 (dd, J=8.4, 1.2 Hz, 1H), 7.56 (dd, J=8.3, 2.0 Hz, 1H), 7.16 (s, 1H), 7.13 (m, 2H), 7.01 (d, J=8.3 Hz, 1H), 6.78-6.71 (m, 1H), 6.63 (t, J=5.9 Hz, 1H), 4.38 (d, J=5.8 Hz, 2H). ¹³C NMR (101 MHz, DMSO) δ 148.4, 145.9, 140.5, 136.6, 135.3, 134.0, 133.8, 133.5, 133.0, 132.6, 132.3, 129.9, 129.1, 124.3, 117.9, 115.9, 113.7, 45.5. HRMS (ESI): calcd for C₂₀H₁₃Cl₂N₂O₄S, (M−H)⁻ 446.9979, found 446.9975. HPLC purity: 95.35%.

### 3-((4-((3,4-Dichlorophenyl)thio)-3-nitrobenzyl)amino)-4-methoxybenzoic acid (C3-072)

The title compound was prepared from the hydrolysis of Methyl 3-((4-((3,4-dichlorophenyl)thio)-3-nitrobenzyl)amino)-4-methoxybenzoate (46d) (30 mg, 0.06 mmol) in 1 M NaOH (0.2 ml) and dioxane (0.6 ml) at 50° C. overnight. Dark yellow solid, 22 mg, 76% yield, mp 235-237° C. ¹H NMR (400 MHz, DMSO) δ 8.19 (s, 1H), 7.92 (d, J=1.8 Hz, 1H), 7.77 (d, J=8.3 Hz, 1H), 7.65-7.48 (m, 2H), 7.22 (d, J=8.2 Hz, 1H), 6.99 (d, J=8.3 Hz, 1H), 6.91 (s, 1H), 6.85 (d, J=8.3 Hz, 1H), 5.90 (s, 1H), 4.41 (d, J=5.9 Hz, 2H), 3.86 (s, 3H). ¹³C NMR (101 MHz, DMSO) δ 169.8, 149.8, 145.8, 140.7, 137.0, 136.6, 135.4, 134.1, 133.6, 133.5, 133.0, 132.6, 132.3, 129.8, 126.7, 124.1, 119.0, 110.4, 109.4, 56.0, 45.4. HRMS (ESI): calcd for C₂₁H₁₃Cl₂N₂O₅S, (M−H)⁻ 474.9928, found 474.9921. HPLC purity: 90.49%.

### 4-Bromo-3-((4-((3,4-dichlorophenyl)thio)-3-nitrobenzyl)amino)benzoic Acid (C3-078)

The title compound was prepared from the hydrolysis of Methyl 4-bromo-3-((4-((3,4-dichlorophenyl)thio)-3-nitrobenzyl)amino)benzoate (48d) (40 mg, 0.07 mmol) in 1 M NaOH (0.3 ml) and dioxane (1.0 ml) at 50° C. overnight. Yellow solid, 35 mg, 90% yield, mp 231-233° C. ¹H NMR (400 MHz, DMSO) δ 8.22 (s, 1H), 7.92 (d, J=1.8 Hz, 1H), 7.77 (d, J=8.3 Hz, 1H), 7.57 (q, 2H), 7.49 (d, J=8.0 Hz, 1H), 7.07 (d, J=8.3 Hz, 1H), 7.01 (d, J=8.1 Hz, 2H), 6.26 (s, 1H), 4.51 (d, J=5.9 Hz, 2H). ¹³C NMR (101 MHz, DMSO) δ168.4, 145.8, 144.8, 139.7, 136.7, 135.4, 134.4, 133.6, 133.5, 133.0, 132.8, 132.6, 132.2, 129.9, 124.1, 118.9, 113.1, 112.2, 45.4. HRMS (ESI): calcd for C₂₀H₁₂BrCl₂N₂O₄S, (M−H)⁻ 524.9084, found 524.9079. HPLC purity: 99.36%.

### 3-((4-((3,4-Dichlorophenyl)thio)-3-nitrobenzyl)amino)-4-fluorobenzoic Acid (C3-082)

The title compound was prepared from the hydrolysis of Methyl 3-((4-((3,4-dichlorophenyl)thio)-3-nitrobenzyl)amino)-4-fluorobenzoate (49d) (30 mg, 0.06 mmol) in 1 M NaOH (0.2 ml) and dioxane (0.6 ml) at 50° C. overnight. Yellow solid, 21 mg, 75% mg, mp 237-239° C. ¹H NMR (400 MHz, DMSO) δ 8.24 (s, 1H), 7.91 (s, 1H), 7.77 (d, J=8.3 Hz, 1H), 7.60 (d, J=8.2 Hz, 1H), 7.56 (d, J=8.2 Hz, 1H), 7.18 (m, 1H), 7.10 (m, 2H), 7.00 (d, J=8.3 Hz, 1H), 6.52 (s, 1H), 4.45 (d, J=5.4 Hz, 2H). ¹³C NMR (101 MHz, DMSO) δ 168.3, 153.7 (d, J=245.4 Hz), 145.8, 134, 136.6, 136.2, 136.1, 135.4, 134.3, 133.6, 133.6, 133.0, 132.6, 132.2, 129.9, 124.2, 118.4 (d, J=8.0 Hz), 114.8 (d, J=18.2 Hz), 113.2 (d, J=4.0 Hz), 45.0. HRMS (ESI): calcd for C₂₀H₁₂C₂FN₂O₄S, (M−H)⁻ 464.9884, found 464.9872. HPLC purity: 100.00%.

### 3-((4-((3,4-Dichlorophenyl)thio)-3-nitrobenzyl)amino)-4-methylbenzoic Acid (C3-075)

The title compound was prepared from the hydrolysis of methyl 3-((4-((3,4-dichlorophenyl)thio)-3-nitrobenzyl)amino)-4-methylbenzoate (50d) (42 mg, 0.09 mmol) in 1 M NaOH (0.3 ml) and dioxane (1.0 ml) at 50° C. overnight. Yellow solid, 27 mg, 65% yield, mp 222-224° C. ¹H NMR (400 MHz, DMSO) δ 8.23 (s, 1H), 7.91 (s, 1H), 7.77 (d, J=8.3 Hz, 1H), 7.58 (m, 2H), 7.09 (m, 2H), 6.99 (d, J=8.3 Hz, 1H), 6.91 (s, 1H), 5.95 (s, 1H), 4.45 (d, J=5.1 Hz, 2H), 2.21 (s, 3H). ¹³C NMR (101 MHz, DMSO) δ 145.9, 145.9, 140.58, 136.6, 135.4, 134.0, 133.6, 133.6, 133.0, 132.6, 132.3, 130.2, 129.9, 127.3, 124.2, 118.1, 110.2, 45.5, 18.4. HRMS (ESI): calcd for C₂₁H₁₅Cl₂N₂O₄S, (M−H)⁻ 461.0135, found 461.0128. HPLC purity: 98.78%.

### 2-Chloro-5-((4-((3,4-dichlorophenyl)thio)-3-nitrobenzyl)amino)benzoic Acid (C3-079)

The title compound was prepared from the hydrolysis of Methyl 2-chloro-5-((4-((3,4-dichlorophenyl)thio)-3-nitrobenzyl)amino)benzoate (51d) (30 mg, 0.06 mmol) in 1 M NaOH (0.2 ml) and dioxane (0.6 ml) at 50° C. overnight. Yellow solid, 22 mg, 76% yield, mp 148-150° C. ¹H NMR (400 MHz, DMSO) δ 8.20 (d, J=1.0 Hz, 1H), 7.92 (d, J=2.0 Hz, 1H), 7.78 (d, J=8.3 Hz, 1H), 7.64-7.47 (m, 2H), 7.00 (d, J=8.3 Hz, 1H), 6.93 (d, J=8.5 Hz, 1H), 6.62 (s, 1H), 6.50-6.34 (m, 2H), 4.31 (d, J=6.0 Hz, 2H). ¹³C NMR (101 MHz, DMSO) δ 146.8, 145.8, 140.5, 136.6, 135.4, 134.1, 133.8, 133.6, 133.0, 132.6, 132.3, 123.0, 129.8, 124.2, 117.4, 113.4, 112.9, 45.5. HRMS (ESI): calcd for C₂₀H₁₂Cl₃N₂O₄S, (M−H)⁻ 480.9589, found 480.9589. HPLC purity: 98.50%.

### 3-((4-((3,4-Dichlorophenyl)thio)-3-nitrobenzyl)amino)-4-morpholinobenzoic Acid (C3-073)

The title compound was prepared from the hydrolysis of Methyl 2-chloro-5-((4-((3,4-dichlorophenyl)thio)-3-nitrobenzyl)amino)benzoate (52d) (35 mg, 0.06 mmol) in 1 M NaOH (0.2 ml) and dioxane (0.6 ml) at 50° C. overnight. Yellow solid, 22 mg, 65% yield, mp 147-149° C. ¹H NMR (400 MHz, DMSO) δ 12.45 (s, 1H), 8.22 (s, 1H), 7.92 (s, 1H), 7.77 (d, J=8.2 Hz, 1H), 7.56 (t, J=5.5 Hz, 2H), 7.24 (d, J=7.7 Hz, 1H), 7.02 (dd, J=12.0, 8.4 Hz, 2H), 6.92 (s, 1H), 5.94 (t, J=5.2 Hz, 1H), 4.48 (d, J=5.1 Hz, 2H), 3.82 (s, 4H), 2.90 (s, 4H). ¹³C NMR (101 MHz, DMSO) δ 145.8, 141.4, 141.3, 140.9, 136.7, 135.5, 134.1, 133.6, 133.4, 133.0, 132.6, 132.2, 129.8, 123.9, 118.9, 118.7, 111.3, 67.0, 51.6, 45.9. HRMS (ESI): calcd for C₂₄H₂₀Cl₂N₃O₅S, (M−H)⁻ 532.0506, found 532.0499. HPLC purity: 95.73%.

### 3-((4-((3,4-Dichlorophenyl)thio)-3-nitrobenzyl)amino)benzamide (C3-066)

The title compound was prepared from 3-((4-Fluoro-3-nitrobenzyl)amino)benzamide (53c, 58 mg, 0.2 mg) and 3,4-dichlorobenzenethiol (51 mg, 0.3 mmol) in a similar manner as described for compound 39d. Yellow solid, 40 mg, 45% yield, mp 158-160° C. ¹H NMR (400 MHz, DMSO) δ 8.23 (s, 1H), 7.91 (s, 1H), 7.77 (d, J=8.3 Hz, 2H), 7.60 (dd, J=8.4, 1.2 Hz, 1H), 7.56 (dd, J=8.3, 1.8 Hz, 1H), 7.18 (s, 1H), 7.10 (t, J=7.7 Hz, 1H), 7.08-7.02 (m, 2H), 7.01 (d, J=8.3 Hz, 1H), 6.70 (d, J=7.4 Hz, 1H), 6.55 (t, J=6.1 Hz, 1H), 4.39 (d, J=6.1 Hz, 2H). ¹³C NMR (101 MHz, DMSO) δ 168.9, 148.5, 145.8, 140.5, 136.6, 135.7, 135.4, 134.1, 133.9, 133.6, 133.0, 132.6, 132.3, 129.8, 129.2, 124.3, 115.8, 115.6, 111.8, 45.3. HRMS (ESI): calcd for C₂₀H₁₄Cl₂N₃O₃S, (M−H)⁻ 446.0138, found 446.0129. HPLC purity: 100.00%.

### (3-((4-((3,4-Dichlorophenyl)thio)-3-nitrobenzyl)amino)phenyl)methanol (C3-080)

The title compound was prepared from (3-((4-Fluoro-3-nitrobenzyl)amino)phenyl)methanol (55c, 85 mg, 0.3 mmol) and 3,4-dichlorobenzenethiol (77 mg, 0.45 mmol) in a similar manner as described for compound 39d. Yellow solid, 100 mg, 77% yield, mp 118-120° C. ¹H NMR (400 MHz, CDCl₃) δ 8.24 (d, J=0.9 Hz, 1H), 7.67 (d, J=2.0 Hz, 1H), 7.55 (d, J=8.3 Hz, 1H), 7.41 (td, J=8.2, 1.8 Hz, 2H), 7.15 (t, J=7.8 Hz, 1H), 6.87 (d, J=8.4 Hz, 1H), 6.73 (d, J=7.5 Hz, 1H), 6.63 (s, 1H), 6.50 (dd, J=8.0, 2.0 Hz, 1H), 4.60 (d, J=5.9 Hz, 2H), 4.41 (d, J=5.8 Hz, 2H), 4.23 (t, J=5.4 Hz, 1H), 1.60 (t, J=6.0 Hz, 1H). ¹³C NMR (101 MHz, CDCl₃) δ 147.5, 145.5, 142.3, 138.5, 136.9, 136.1, 134.7, 134.6, 134.0, 132.5, 131.9, 131.5, 129.6, 128.9, 124.3, 116.8, 112.1, 111.4, 65.4, 46.8. HRMS (ESI): calcd for C₂₀H₁₅Cl₂N₂O₃S, (M−H)⁻ 433.0186, found 433.0184. HPLC purity: 96.19%.

### 4-Chloro-2-((4-((3,4-dichlorophenyl)thio)-3-nitrobenzyl)amino)benzoic Acid (C3-087)

The title compound was prepared from the hydrolysis of Methyl 4-chloro-2-((4-((3,4-dichlorophenyl)thio)-3-nitrobenzyl)amino)benzoate (56d) (30 mg, 0.06 mmol) in 1 M NaOH (0.2 ml) and dioxane (0.8 ml) at 50° C. overnight. Yellow solid, 20 mg, 69% yield, mp 220-222° C. ¹H NMR (400 MHz, DMSO) δ 12.95 (s, 1H), 8.54 (s, 1H), 8.23 (s, 1H), 7.93 (d, J=1.8 Hz, 1H), 7.79 (m, 2H), 7.58 (m, 2H), 7.02 (d, J=8.4 Hz, 1H), 6.69 (d, J=1.3 Hz, 1H), 6.60 (dd, J=8.5, 1.6 Hz, 1H), 4.59 (s, 2H). ¹³C NMR (101 MHz, DMSO) δ 169.9, 151.5, 145.8, 139.4, 139.2, 136.7, 135.5, 134.6, 134.0, 133.6, 133.0, 132.6, 132.2, 130.0, 124.4, 115.3, 111.3, 110.9, 44.6. HRMS (ESI): calcd for C₂₀H₁₂Cl₃N₂O₄S, (M−H)⁻ 480.9589, found 480.9579. HPLC purity: 95.83%.

### 2-Chloro-6-((4-((3,4-Dichlorophenyl)thio)-3-nitrobenzyl)amino)benzoic Acid (C3-088)

The title compound was prepared from 2-Chloro-6-((4-fluoro-3-nitrobenzyl)amino)benzoic Acid (57c, 120 mg, 0.5 mmol) and 3,4-Dichlorobenzenethiol (107 mg, 0.6 mmol) in a similar manner as described for compound 39d. Yellow solid, 90 mg, 37% yield, mp 204-206° C. ¹H NMR (400 MHz, DMSO) δ 13.47 (s, 1H), 8.21 (s, 1H), 7.91 (d, J=1.9 Hz, 1H), 7.78 (d, J=8.3 Hz, 1H), 7.56 (d, J=8.3 Hz, 2H), 7.09 (t, J=8.2 Hz, 1H), 7.00 (d, J=8.3 Hz, 1H), 6.65 (d, J=7.8 Hz, 1H), 6.47 (d, J=8.4 Hz, 1H), 4.46 (s, 2H). ¹³C NMR (101 MHz, DMSO) δ 168.0, 147.2, 145.8, 139.8, 136.6, 135.4, 134.3, 133.6, 133.0, 132.6, 132.2, 131.8, 131.6, 129.8, 124.3, 118.7, 117.4, 110.4, 45.2. HRMS (ESI): calcd for C₂₀H₁₂Cl₃N₂O₄S, (M−H)⁻ 480.9589, found 480.9579. HPLC purity: 95.12%.

### 2-((4-((3,4-Dichlorophenyl)thio)-3-nitrobenzyl)amino)benzamide (C3-090)

The title compound was prepared from 2-((4-Fluoro-3-nitrobenzyl)amino)benzamide (58c) (58 mg, 0.2 mmol) and 3,4-Dichlorobenzenethiol (43 mg, 0.24 mmol) in a similar manner as described for compound 39d. Yellow solid, 37 mg, 41% yield, mp 184-186° C. ¹H NMR (400 MHz, DMSO) δ 8.68 (t, J=6.0 Hz, 1H), 8.19 (s, 1H), 7.92 (d, J=1.9 Hz, 1H), 7.88 (s, 1H, C(═O)NH, 7.78 (d, J=8.3 Hz, 1H), 7.62 (d, J=7.3 Hz, 1H), 7.60-7.54 (m, 2H), 7.22 (s, C(═O)NH 1H), 7.19 (t, J=7.7 Hz, 1H), 7.16-7.16 (m, 1H), 7.01 (d, J=8.3 Hz, 1H), 6.62-6.50 (m, 2H), 4.49 (d, J=6.1 Hz, 2H). ¹³C NMR (101 MHz, DMSO) δ 172.0, 149.5, 145.8, 140.2, 136.6, 135.4, 134.3 133.7, 133.6, 133.0, 133.0, 132.6, 132.3, 130.0, 129.6, 124.2, 115.2, 115.1, 112.0, 45.0. HRMS (ESI): calcd for C₂₀H₁₆Cl₂N₃O₃S, (M+H)⁺ 448.0284, found 448.0276. HPLC purity: 100.00%.

### 2-((4-((3,4-Dichlorophenyl)thio)-3-nitrobenzyl)amino)benzonitrile (C3-091)

The title compound was prepared from 2-((4-fluoro-3-nitrobenzyl)amino)benzonitrile (59c) (54 mg, 0.2 mmol) and 3,4-Dichlorobenzenethiol (43 mg, 0.24 mmol) in a similar manner as described for compound 39d. Yellow solid, 50 mg, 58% yield, mp 173-175° C. ¹H NMR (400 MHz, CDCl₃) δ 8.24 (s, 1H), 7.70 (d, J=1.8 Hz, 1H), 7.57 (d, J=8.2 Hz, 1H), 7.44 (m, 3H), 7.36 (t, J=7.8 Hz, 1H), 6.90 (d, J=8.4 Hz, 1H), 6.76 (t, J=7.5 Hz, 1H), 6.54 (d, J=8.5 Hz, 1H), 5.13 (t, J=5.3 Hz, 1H), 4.51 (d, J=5.8 Hz, 2H). ¹³C NMR (101 MHz, CDCl₃) δ 149.3, 145.4, 137.0, 137.0, 136.6, 134.9, 134.7, 134.4, 134.1, 133.0, 132.1, 131.9, 131.2, 129.1, 124.3, 117.8, 117.6, 110.9, 96.6, 46.2. HRMS (ESI): calcd for C₂₀H₁₂Cl₂N₃O₂S, (M−H)⁻ 429.0106, found, 428.0033. HPLC purity: 100.00%.

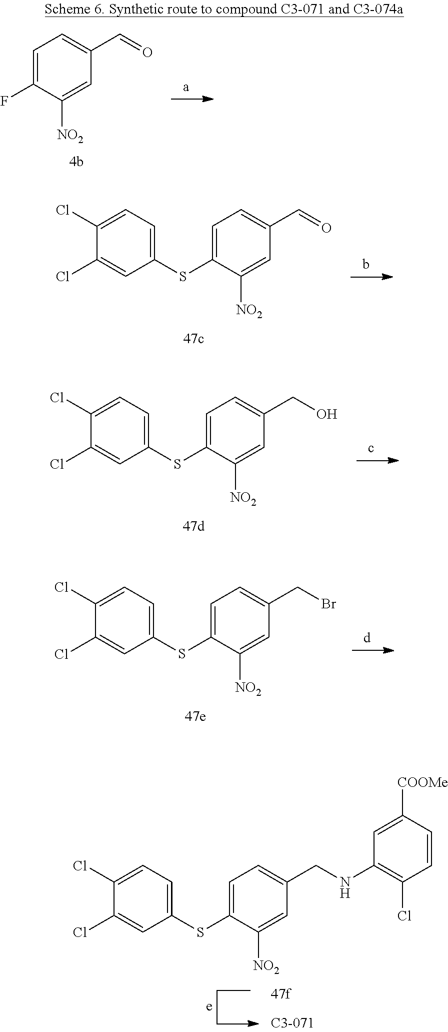

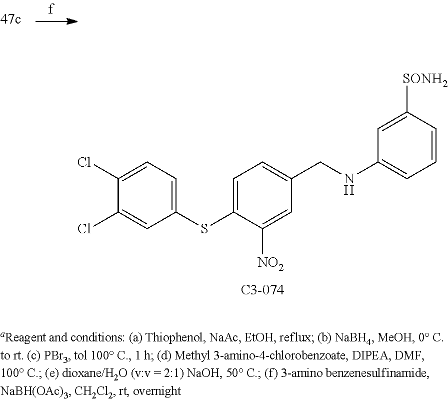

### 4-((3,4-Dichlorophenyl)thio)-3-nitrobenzaldehyde (47c)

The title compound was prepared from Methyl 3-((4-fluoro-3-nitrobenzyl)amino)benzoate (4b, 169 mg, 1 mmol) and 3,4-Dichlorobenzenethiol (214 mg, 1.2 mmol) in a similar manner as described for compound 39d; yellow solid, 246 mg, 75% yield. ¹H NMR (400 MHz, CDCl₃) δ 10.02 (s, 1H), 8.75 (d, J=1.4 Hz, 1H), 7.90 (dd, J=8.5, 1.5 Hz, 1H), 7.74 (d, J=1.9 Hz, 1H), 7.64 (d, J=8.3 Hz, 1H), 7.46 (dd, J=8.2, 1.9 Hz, 1H), 7.04 (d, J=8.4 Hz, 1H).

### (4-((3,4-Dichlorophenyl)thio)-3-nitrophenyl)methanol (47d)

In a dried round bottom flask, 4-((3,4-Dichlorophenyl)thio)-3-nitrobenzaldehyde (47c) (100 mg, 0.3 mmol) and MeOH (5 ml) was cooled to 0° C. with ice-water bath. NaBH₄ (23 mg, 0.6 mmol) was added and the reaction mixture was allowed to stir at room temperature for another 1 h. NH4Cl aqueous solution was added to quench the reaction and the aqueous phase was extracted with ether acetate. The organic phase was combined and washed with saturated brine, dried with Na₂SO₄ and purified by column chromatography to generate the titled compound as pale yellow solid, 87 mg, 88% yield. ¹H NMR (400 MHz, CDCl₃) δ 8.26 (s, 1H), 7.69 (d, J=2.0 Hz, 1H), 7.57 (d, J=8.3 Hz, 1H), 7.43 (td, J=8.2, 1.8 Hz, 2H), 6.92 (d, J=8.4 Hz, 1H), 4.77 (d, J=5.7 Hz, 2H).

### (4-(Bromomethyl)-2-nitrophenyl)(3,4-dichlorophenyl)sulfane (47e)

Phosphorus tribromide (20 ul, 0.22 mmol) was added to a stirred solution of (4-((3,4-Dichlorophenyl)thio)-3-nitrophenyl)methanol (47d) (60 mg, 0.18 mmol) in Toluene (2 ml) at 40° C. The solution was heated to 100° C. for 30 min. After completion of the reaction, the contents were cooled to ambient temperature. The liquid was diluted with ether acetate and washed with water and brine, dried with Na₂SO₄ and purified by column chromatography to generate the titled compound as yellow solid, 58 mg, 82% yield. ¹H NMR (400 MHz, CDCl₃) δ 8.29 (d, J=1.8 Hz, 1H), 7.71 (d, J=2.0 Hz, 1H), 7.59 (d, J=8.3 Hz, 1H), 7.44 (ddd, J=8.2, 4.2, 2.0 Hz, 2H), 6.88 (d, J=8.4 Hz, 1H), 4.49 (s, 2H).

### Methyl 4-chloro-3-((4-((3,4-dichlorophenyl)thio)-3-nitrobenzyl)amino)benzoate (47f)

To a flask was added (4-(bromomethyl)-2-nitrophenyl)(3,4-dichlorophenyl)sulfane (118 mg, 0.3 mmol), methyl 3-amino-4-chlorobenzoate (56 mg, 0.3 mmol), DIPEA (149 ul, 0.9 mmol) and DMF 2 ml was heated to 100° C. for 4 h. The reaction mixture was cooled to ambient and water was added, followed by extracting with EA. The organic phase was combined and washed with saturated brine, dried with Na₂SO₄ and purified by column chromatography to generate the titled compound as yellow solid, 32 mg, 21% yield. ¹H NMR (400 MHz, DMSO) δ 8.25 (d, J=1.2 Hz, 1H), 7.91 (d, J=1.9 Hz, 1H), 7.78 (d, J=8.3 Hz, 1H), 7.59 (d, J=7.6 Hz, 1H), 7.57 (dd, J=8.4, 2.0 Hz, 1H), 7.42 (d, J=8.2 Hz, 1H), 7.16 (dd, J=8.2, 1.6 Hz, 1H), 7.06 (d, J=1.6 Hz, 1H), 7.02 (d, J=8.4 Hz, 1H), 6.61 (t, J=6.1 Hz, 1H), 4.53 (d, J=6.1 Hz, 2H), 3.76 (s, 3H).

### 4-Chloro-3-((4-((3,4-dichlorophenyl)thio)-3-nitrobenzyl)amino)benzoic Acid (C3-071)

The title compound was prepared from the hydrolysis of Methyl 4-chloro-3-((4-((3,4-dichlorophenyl)thio)-3-nitrobenzyl)amino)benzoate (47f) (30 mg, 0.06 mmol) in 1 M NaOH (0.2 ml) and dioxane (0.6 ml) at 50° C. overnight. Yellow solid, 17 mg, 59% yield, mp 196-198° C. ¹H NMR (400 MHz, DMSO) δ 8.21 (s, 1H), 7.92 (s, 1H), 7.77 (d, J=8.3 Hz, 1H), 7.57 (d, J=8.2 Hz, 2H), 7.28 (d, J=8.1 Hz, 1H), 7.14 (d, J=8.0 Hz, 1H), 7.07 (s, 1H), 6.99 (d, J=8.3 Hz, 1H), 6.34 (s, 1H), 4.49 (d, J=5.9 Hz, 2H). ¹³C NMR (101 MHz, DMSO) δ 167.7, 145.8, 143.8, 139.7, 136.6, 135.4, 134.4, 133.6, 133.5, 133.0, 132.6, 132.2, 131.7, 123.0, 129.6, 124.2, 122.7, 118.3, 112.0, 45.1. HRMS (ESI): calcd for C₂₀H₁₂Cl₃N₂O₄S, (M−H)⁻ 480.9589, found 480.9577. HPLC purity: 97.49%.

### 3-((4-((3,4-Dichlorophenyl)thio)-3-nitrobenzyl)amino)benzenesulfonamide (C3-074)

In a dried round bottom flask, 4-((3,4-dichlorophenyl)thio)-3-nitrobenzaldehyde (47c) (50 mg, 0.15 mmol), 3-Aminobenzenesulfinamide (23 mg, 0.15 mmol) and CH₂Cl₂ (5 ml) were stirred at room temperature for 1 h. Then the reaction was cooled down to 0° C. and NaBH(OAc)₃ (95 mg, 0.45 mmol) was added in two portions over 1 min. The reaction was allowed to stir from 0° C. to room temperature overnight. The reaction was quenched with a saturated aqueous solution of NH₄Cl and then extracted with CH₂Cl₂. The combined organic layers were dried with Na₂SO₄, filtered and the solvent was removed under reduced pressure. The crude was purified by column chromatography to give the title compound, yellow solid, 21 mg, 29% yield, mp 191-193° C. ¹H NMR (400 MHz, DMSO) δ 8.24 (s, 1H), 7.91 (s, 1H), 7.79 (d, J=8.3 Hz, 1H), 7.58 (dd, J=15.2, 8.4 Hz, 2H), 7.21 (t, J=8.0 Hz, 1H), 7.17 (s, 2H), 7.05 (s, 1H), 7.00 (dd, J=11.9, 8.4 Hz, 2H), 6.87 (t, J=5.7 Hz, 1H), 6.71 (d, J=7.8 Hz, 1H), 4.39 (d, J=5.7 Hz, 2H). ¹³C NMR (101 MHz, DMSO) δ 148.8, 145.8, 145.3, 134.0, 136.6, 135.4, 134.3, 133.9, 133.6, 133.0, 132.6, 132.3, 130.0, 129.9, 124.4, 115.2, 113.6, 109.7, 45.3. HRMS (ESI): calcd for C₁₉H₁₄Cl₂N₃O₄S2, (M−H)⁻ 481.9808, found 481.9801. HPLC purity: 97.55%.

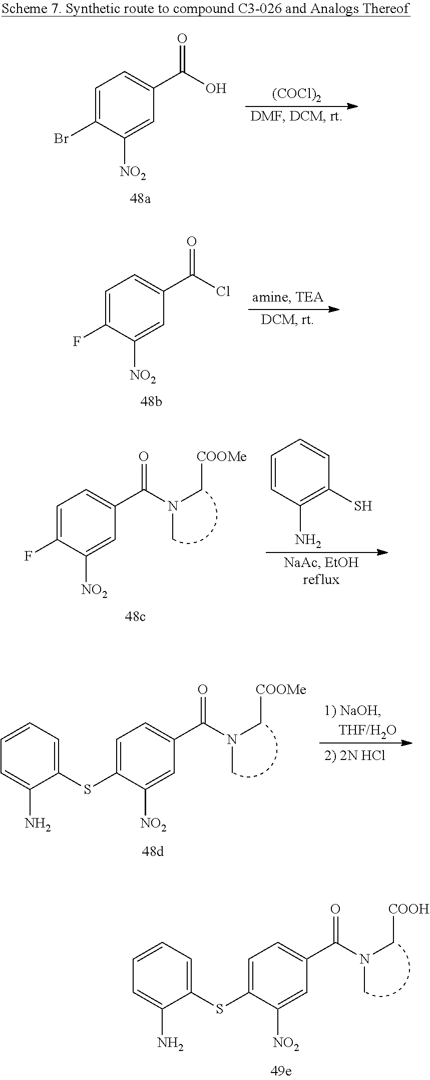

Synthesis of Compound 48c

To a solution of 4-fluro-3-nitrobenzoic acid 48a (1 mmol) in 3 ml DCM was added a solution of oxalyl dichloride (3 mmol) in 2 ml DCM at 0° C. and then a catalytic amount of DMF was added. Then reaction mixture was allowed to warm to rt. gradually and stirred for another two hours. After that, the solvent was removed in vacuo. DCM was added, and the solvent was removed again. The brown oil residue was immediately dissolved in DCM. This solution was slowly added to a solution of the corresponding amine (1 mmol) and TEA (2 mmol) at 0° C. The mixture was stirred overnight at rt. Water was added, and the aqueous phase was extracted with DCM and dried by Na₂SO₄, purified by chromatograph to get compound 48c.

Synthesis of Compound 48d

To a flask was added compound 48c (0.2 mmol), 2-Aminobenzenethiol (0.24 mmol), NaAc (1 mmol) and EtOH 5 ml. The mixture stirred at rt. overnight. Then water was added and then the aqueous was extracted by EA. The combined organic layers were dried over Na₂SO₄. Purified by chromatography to provide compound 48d as yellow solid.

General Procedure for the Synthesis of Compound 49e

The methyl ester of compound 48d (1 eq.) was hydrolyzed with NaOH (5 eq. in H₂O/THF=1:1 ml) at room temperature. After completing, THF was removed under reduced pressure and the remained aqueous phase was washed by DCM. Subsequently, the water solution was acidified by 2N HCl. Yellow solid was precipitated and collected by filtration, washed by H₂O, dried in vacuum.

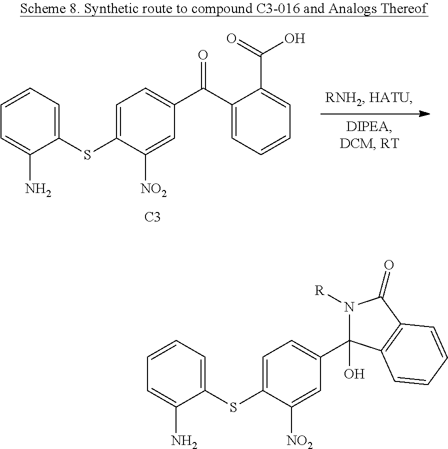

General Procedure for the Synthesis

Amine (1 eq.) was added to a solution of Compound C3 (1 eq.) and HATU (1.5 eq.) in DCM, then DIPEA (3 eq) was added. The resulting mixture was stirred overnight at room temperature. The reaction was quenched with water and extracted with EA. The combined organic layers were washed with 2N HCl solution, NaHCO₃ solution and NaCl aq., dried with sodium sulfate, and the residue obtained upon evaporation of solvent was purified by column chromatography.

General Procedure for the Antimicrobial Assay

The antimicrobial activity of the compounds was determined by broth microdilution according to the Clinical & Laboratory Standards Institute (CLSI) guidelines. The test medium was cation-adjusted Mueller-Hinton broth (MH). Serial two-fold dilutions were performed for the tested chemicals starting from 256 μg/mL to 0.5 μg/mL, and the bacterial cell inoculum was adjusted to approximately 5×10⁵ CFU/mL. Results were taken after 20 h of incubation at 37° C. The MIC was defined as the lowest concentration of antibiotic with no visible growth. Experiments were performed in duplicates.

What is claimed is:

1. A compound of Formula 1: 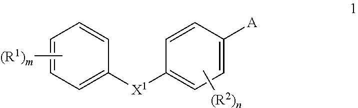
or a pharmaceutically acceptable salt, solvate, or tautomer thereof, wherein
A represents a moiety selected from the group consisting of: 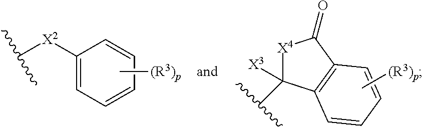
m is a whole number selected from 1-5;
n is a whole number selected from 1-4;
p is a whole number selected from 1-4;
q is a whole number selected from 2-5;
X¹ is —S—, —O—, —C(R)₂—, or —N(R)—;
X² is —S—, —O—, —C(R)₂—, —N(R)—, —C(O)—, —C(S)—, —C(O)C(R)₂—*, —C(R)₂C(O)—*, —N═CH—*, —(CR₂)_{q}—, —CH═N—*, —(R)C═C(R)—*, —C≡C—*, —NRC(R)₂—*, —CR₂N(R)—*, —OC(R)₂—*, —C(R)₂—O—*, —SC(R)₂—*, —C(R)₂S—*, —N(R)C(O)—*, —C(O)N(R)—*, —CR(OH)C(R)₂—*, —CR₂CR(OH)—*, —S(O)C(R)₂—*, —S(O)₂C(R)₂—*, —C(R)₂S(O)—*, —C(R)₂S(O)₂—*, —S(O)CR(OH)—*, —S(O)₂CR(OH)—*, —CR(OH)S(O)—*, —CR(OH)S(O)₂—*, —S(O)N(R)—*, —S(O)₂N(R)—*, —N(R)S(O)—*, —NRS(O)₂—*, —S(O)NR(OH)—*, —S(O)₂NR(OH)—*, —NR(OH)S(O)—*, or —NR(OH)S(O)₂—*, wherein * indicates the position of a covalent bond with the moiety: 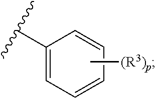
X³ is hydrogen or —OR;
X⁴ is —O—, —N(OR⁴)—, or —N(R⁴)—;
R for each instance is independently selected from the group consisting of hydrogen, alkyl, haloalkyl, alkenyl, alkynyl, cycloalkyl, heterocycloalkyl, aryl, heteroaryl, or aralkyl; or two instances of R together with the atom they are covalently bonded form a 3-6 membered cycloalkyl and heterocyloalkyl;
R¹ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;
R² for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)—N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl;
R³ for each instance is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; and
R⁴ is selected from the group consisting of hydrogen, alkyl, haloalkyl, alkenyl, alkynyl, cycloalkyl, heterocycloalkyl, aryl, heteroaryl, aralkyl, or —(CR₂)ₜY, wherein t is 1-10 and Y is halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkenyl, alkynyl, cycloalkyl, heterocycloalkyl, aryl, heteroaryl, and aralkyl,
with the proviso that the compound of Formula 1 does not have the structure:

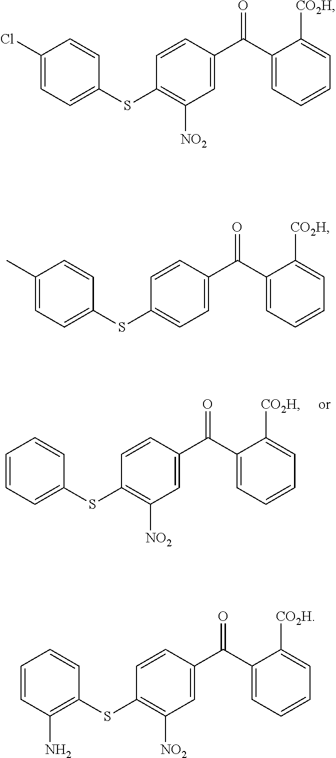 [SMILES: O=C(O)c1ccccc1C(=O)c1ccc(Sc2ccc(Cl)cc2)c([N+](=O)[O-])c1 ; Cc1ccc(Sc2ccc(C(=O)c3ccccc3C(=O)O)cc2)cc1 ; O=C(O)c1ccccc1C(=O)c1ccc(Sc2ccccc2)c([N+](=O)[O-])c1 ; Nc1ccccc1Sc1ccc(C(=O)c2ccccc2C(=O)O)cc1[N+](=O)[O-]]

2. The compound of claim 1, wherein X¹ is —S— or —O—.

3. The compound of claim 1, wherein X² is —S—, —O—, —C(R)₂—, —C(O)—, —C(O)C(R)₂—*, —(R)C═C(R)—*, —CR₂N(R)—*, or —S(O)₂N(R)—*.

4. The compound of claim 1, wherein the compound has the Formula 2: 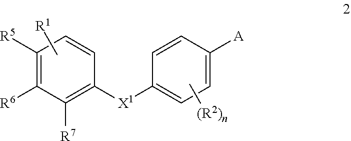
wherein each of R⁵ and R⁶ is independently selected from the group consisting of halide, —N(R)₂, —N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, and aralkyl; each of R⁵ and R⁷ is independently selected from the group consisting of halide, —N(R)₂, —N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, and aralkyl; or R⁷ is halide, —N(R)₂, N(R)C(O)R, —N(R)C(O)OR, —N(R)C(O)N(R)₂, —N(R)S(O)₂R, perhaloalkoxyl, alkyl, haloalkyl, heterocycloalkyl, aryl, heteroaryl, or aralkyl.

5. The compound of claim 1, wherein A is: 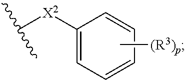
and
X² is —S—, —O—, —CH₂—, —C(O)—, —C(O)CH₂—*, —(R)C═C(R)—*, —CR₂N(R)—*, or —S(O)₂N(R)—*.

6. The compound of claim 5, wherein the compound has the Formula 3: 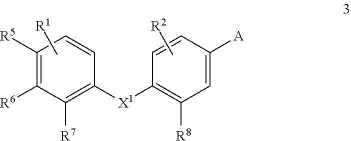
wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl; and
R⁸ is hydrogen, nitro, —N(R)₂, —N(R)C(O)R, —N(R)C(O)OR, —N(R)C(O)N(R)₂, —N(R)S(O)₂R, or perhaloalkoxyl, wherein at least one of R⁵, R⁶, and R⁷ is not hydrogen.

7. The compound of claim 6, wherein A is 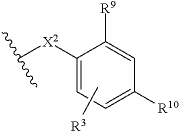
wherein each of R⁹ and R¹⁰ is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; X² is —S—, —O—, —C(R)₂—, —(R)C═C(R)—*, —CR₂N(R)—*, or —S(O)₂N(R)—*, wherein at least one of R⁹ and R¹⁰ is —C(O)OH.

8. The compound of claim 7, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

9. The compound of claim 1, wherein A is: 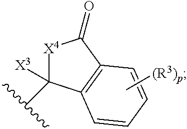
and
X³ is —OR; and X⁴ is —N(OR⁴)— or —N(R⁴)—.

10. The compound of claim 9, wherein X³ is —OH.

11. The compound of claim 10, wherein the compound has the Formula 3: 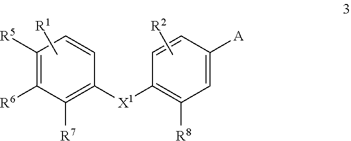
wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl; and
R⁸ is hydrogen, nitro, —N(R)₂, —N(R)C(O)R, —N(R)C(O)OR, —N(R)C(O)N(R)₂, —N(R)S(O)₂R, or perhaloalkoxyl, wherein at least one of R⁵, R⁶, and R⁷ is not hydrogen.

12. The compound of claim 11, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

13. The compound of claim 12, wherein X⁴ is —N(OR⁴)— and R⁴ is hydrogen, alkyl, or cycloalkyl; or X⁴ is —N(R⁴)— and R⁴ is hydrogen, alkyl, cycloalkyl, aryl, or —(CR₂)ₜY, wherein t is 2-4 and Y is —OR, —SR, —N(R)₂, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, or —N(R)S(O)₂R.

14. The compound of claim 1, wherein the compound of Formula 1 is selected from the group consisting of:

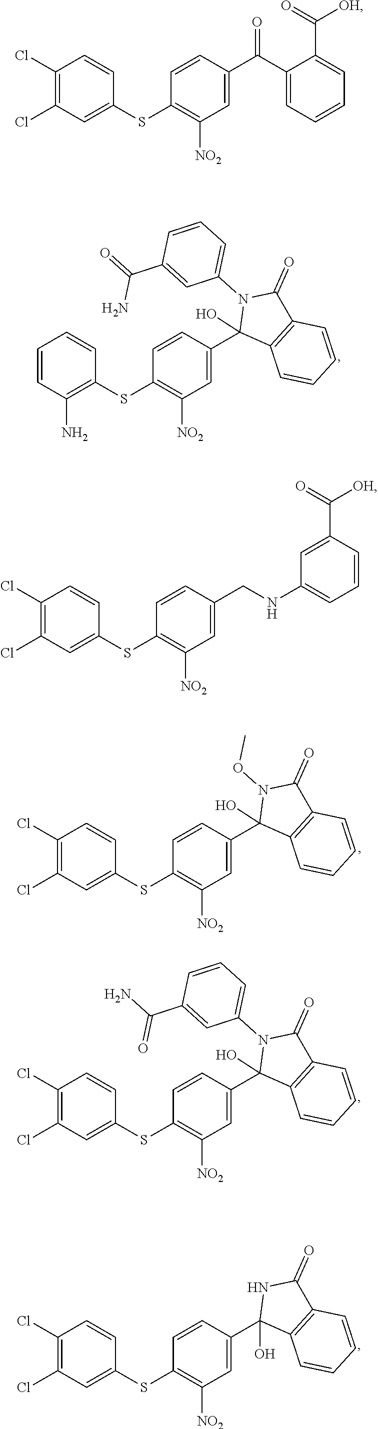

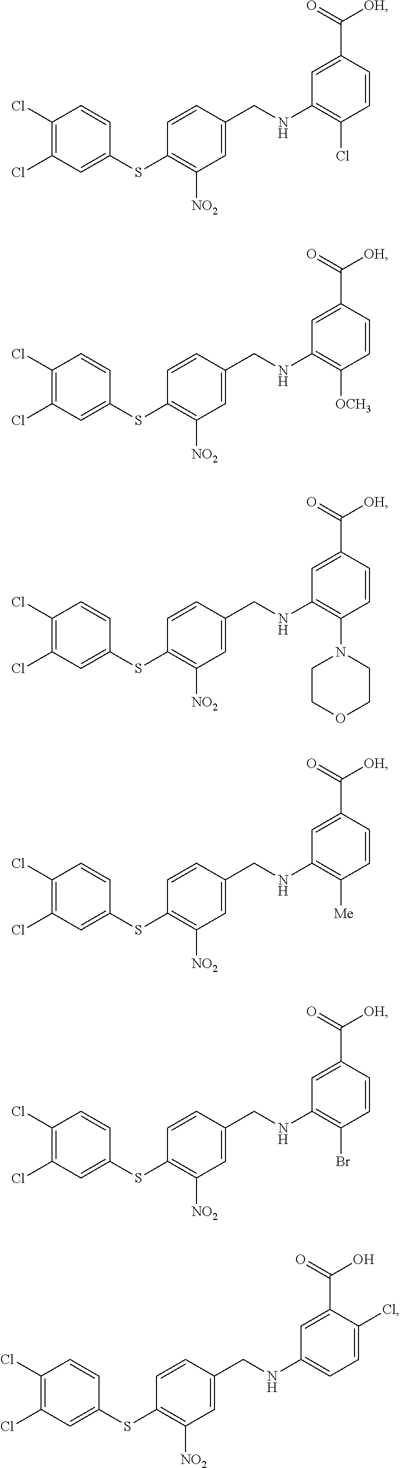 [SMILES: O=C(O)c1ccc(Cl)c(NCc2ccc(Sc3ccc(Cl)c(Cl)c3)c([N+](=O)[O-])c2)c1 ; COc1ccc(C(=O)O)cc1NCc1ccc(Sc2ccc(Cl)c(Cl)c2)c([N+](=O)[O-])c1 ; O=C(O)c1ccc(N2CCOCC2)c(NCc2ccc(Sc3ccc(Cl)c(Cl)c3)c([N+](=O)[O-])c2)c1 ; Cc1ccc(C(=O)O)cc1NCc1ccc(Sc2ccc(Cl)c(Cl)c2)c([N+](=O)[O-])c1 ; O=C(O)c1ccc(Br)c(NCc2ccc(Sc3ccc(Cl)c(Cl)c3)c([N+](=O)[O-])c2)c1 ; O=C(O)c1cc(NCc2ccc(Sc3ccc(Cl)c(Cl)c3)c([N+](=O)[O-])c2)ccc1Cl]

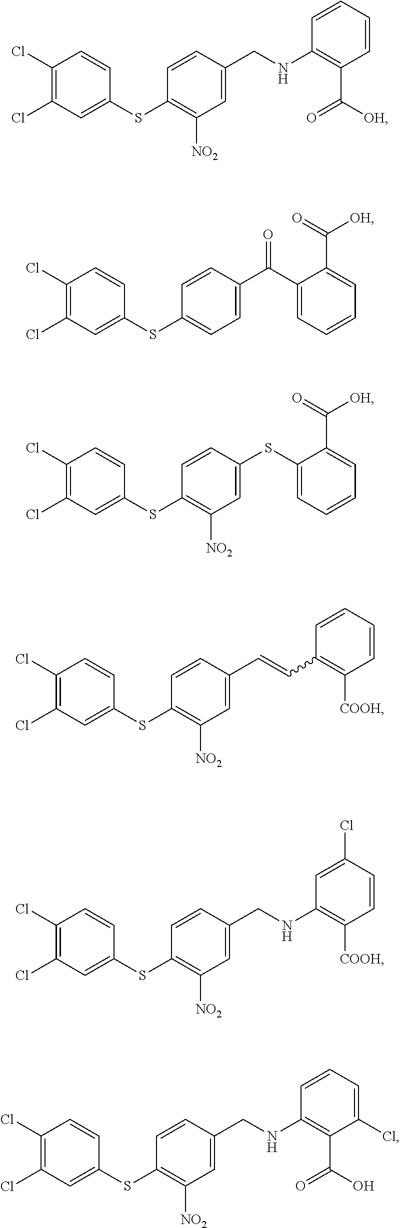 [SMILES: O=C(O)c1ccccc1NCc1ccc(Sc2ccc(Cl)c(Cl)c2)c([N+](=O)[O-])c1 ; O=C(O)c1ccccc1C(=O)c1ccc(Sc2ccc(Cl)c(Cl)c2)cc1 ; O=C(O)c1ccccc1Sc1ccc(Sc2ccc(Cl)c(Cl)c2)c([N+](=O)[O-])c1 ; O=C(O)c1ccccc1C=Cc1ccc(Sc2ccc(Cl)c(Cl)c2)c([N+](=O)[O-])c1 ; O=C(O)c1ccc(Cl)cc1NCc1ccc(Sc2ccc(Cl)c(Cl)c2)c([N+](=O)[O-])c1 ; O=C(O)c1c(Cl)cccc1NCc1ccc(Sc2ccc(Cl)c(Cl)c2)c([N+](=O)[O-])c1]

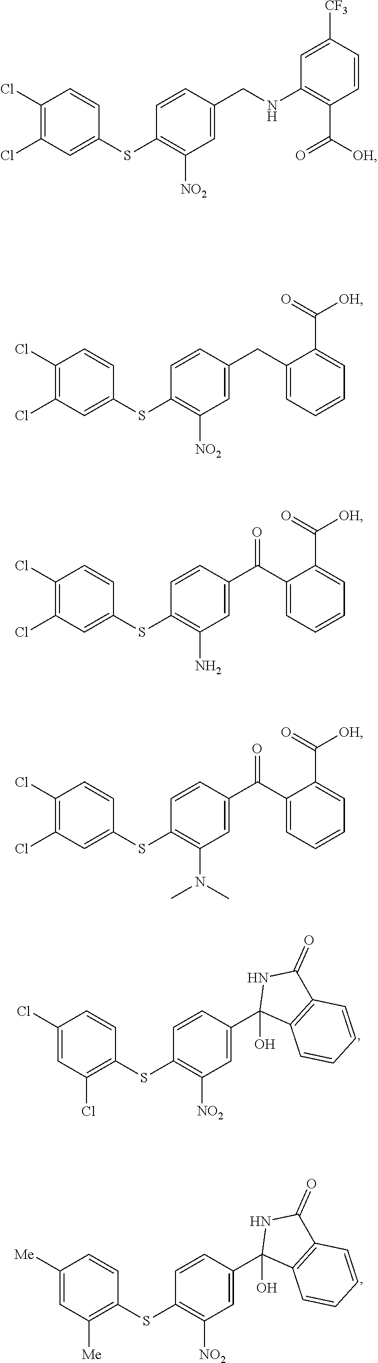 [SMILES: O=C(O)c1ccc(C(F)(F)F)cc1NCc1ccc(Sc2ccc(Cl)c(Cl)c2)c([N+](=O)[O-])c1 ; O=C(O)c1ccccc1Cc1ccc(Sc2ccc(Cl)c(Cl)c2)c([N+](=O)[O-])c1 ; Nc1cc(C(=O)c2ccccc2C(=O)O)ccc1Sc1ccc(Cl)c(Cl)c1 ; CN(C)c1cc(C(=O)c2ccccc2C(=O)O)ccc1Sc1ccc(Cl)c(Cl)c1 ; O=C1NC(O)(c2ccc(Sc3ccc(Cl)cc3Cl)c([N+](=O)[O-])c2)c2ccccc21 ; Cc1ccc(Sc2ccc(C3(O)NC(=O)c4ccccc43)cc2[N+](=O)[O-])c(C)c1]

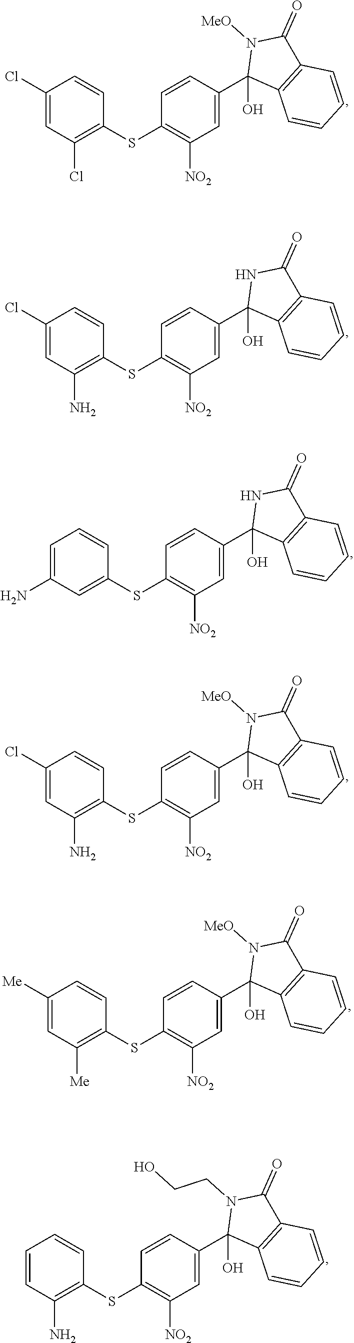 [SMILES: CON1C(=O)c2ccccc2C1(O)c1ccc(Sc2ccc(Cl)cc2Cl)c([N+](=O)[O-])c1 ; Nc1cc(Cl)ccc1Sc1ccc(C2(O)NC(=O)c3ccccc32)cc1[N+](=O)[O-] ; Nc1cccc(Sc2ccc(C3(O)NC(=O)c4ccccc43)cc2[N+](=O)[O-])c1 ; CON1C(=O)c2ccccc2C1(O)c1ccc(Sc2ccc(Cl)cc2N)c([N+](=O)[O-])c1 ; CON1C(=O)c2ccccc2C1(O)c1ccc(Sc2ccc(C)cc2C)c([N+](=O)[O-])c1 ; Nc1ccccc1Sc1ccc(C2(O)c3ccccc3C(=O)N2CCO)cc1[N+](=O)[O-]]

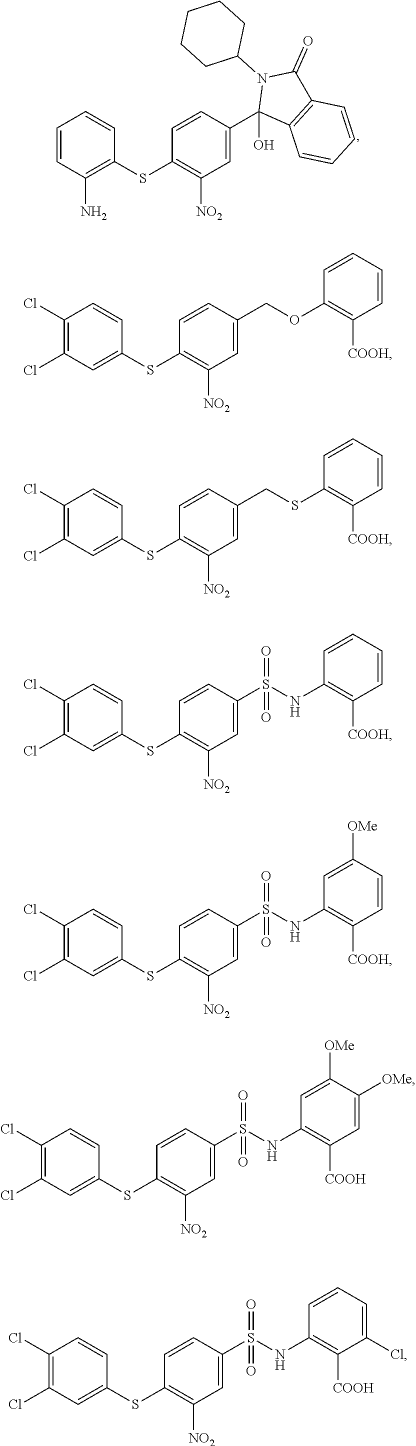

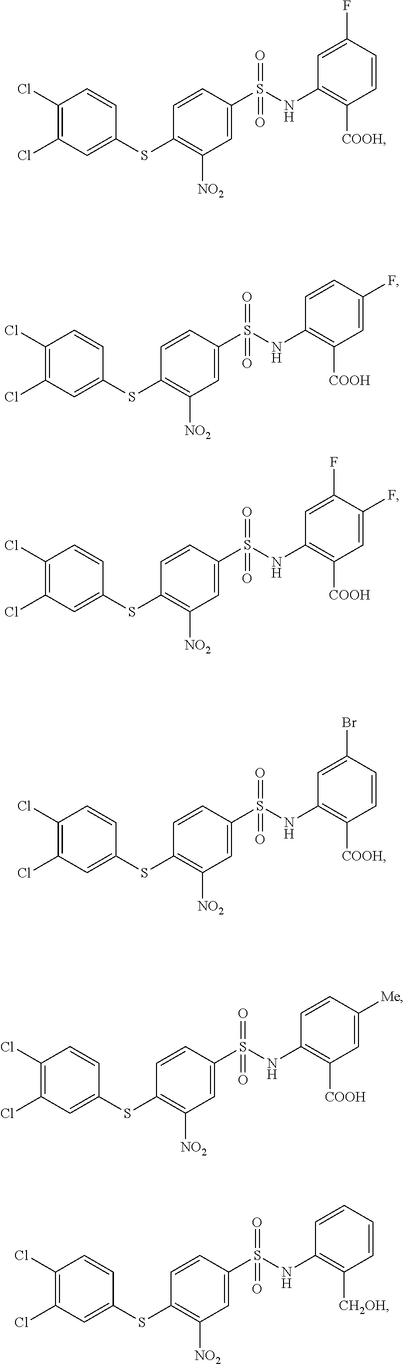 [SMILES: O=C(O)c1ccc(F)cc1NS(=O)(=O)c1ccc(Sc2ccc(Cl)c(Cl)c2)c([N+](=O)[O-])c1 ; O=C(O)c1cc(F)ccc1NS(=O)(=O)c1ccc(Sc2ccc(Cl)c(Cl)c2)c([N+](=O)[O-])c1 ; O=C(O)c1cc(F)c(F)cc1NS(=O)(=O)c1ccc(Sc2ccc(Cl)c(Cl)c2)c([N+](=O)[O-])c1 ; O=C(O)c1ccc(Br)cc1NS(=O)(=O)c1ccc(Sc2ccc(Cl)c(Cl)c2)c([N+](=O)[O-])c1 ; Cc1ccc(NS(=O)(=O)c2ccc(Sc3ccc(Cl)c(Cl)c3)c([N+](=O)[O-])c2)c(C(=O)O)c1 ; O=[N+]([O-])c1cc(S(=O)(=O)Nc2ccccc2CO)ccc1Sc1ccc(Cl)c(Cl)c1]

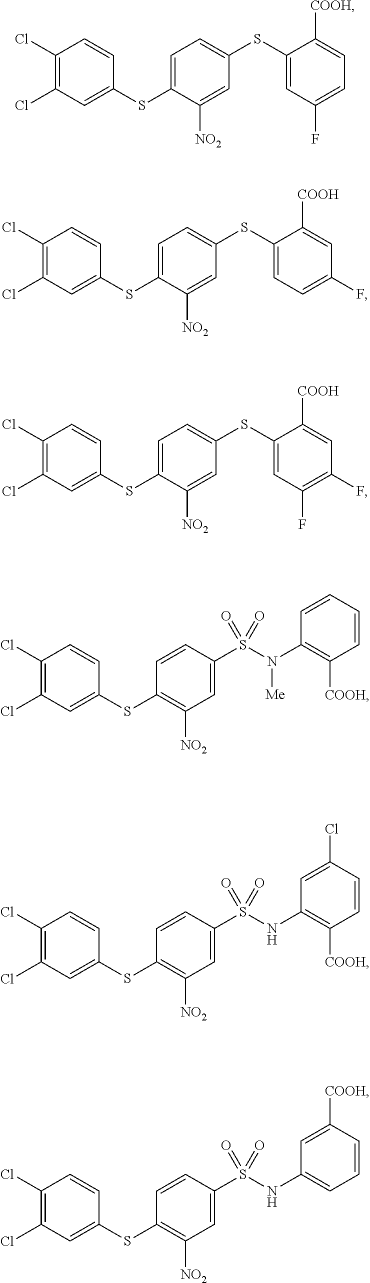 [SMILES: O=C(O)c1ccc(F)cc1Sc1ccc(Sc2ccc(Cl)c(Cl)c2)c([N+](=O)[O-])c1 ; O=C(O)c1cc(F)ccc1Sc1ccc(Sc2ccc(Cl)c(Cl)c2)c([N+](=O)[O-])c1 ; O=C(O)c1cc(F)c(F)cc1Sc1ccc(Sc2ccc(Cl)c(Cl)c2)c([N+](=O)[O-])c1 ; CN(c1ccccc1C(=O)O)S(=O)(=O)c1ccc(Sc2ccc(Cl)c(Cl)c2)c([N+](=O)[O-])c1 ; O=C(O)c1ccc(Cl)cc1NS(=O)(=O)c1ccc(Sc2ccc(Cl)c(Cl)c2)c([N+](=O)[O-])c1 ; O=C(O)c1cccc(NS(=O)(=O)c2ccc(Sc3ccc(Cl)c(Cl)c3)c([N+](=O)[O-])c2)c1]

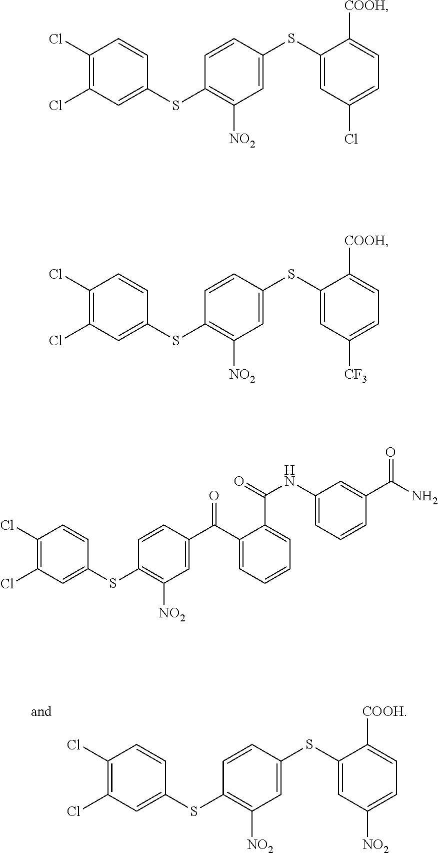 [SMILES: O=C(O)c1ccc(Cl)cc1Sc1ccc(Sc2ccc(Cl)c(Cl)c2)c([N+](=O)[O-])c1 ; O=C(O)c1ccc(C(F)(F)F)cc1Sc1ccc(Sc2ccc(Cl)c(Cl)c2)c([N+](=O)[O-])c1 ; NC(=O)c1cccc(NC(=O)c2ccccc2C(=O)c2ccc(Sc3ccc(Cl)c(Cl)c3)c([N+](=O)[O-])c2)c1 ; O=C(O)c1ccc([N+](=O)[O-])cc1Sc1ccc(Sc2ccc(Cl)c(Cl)c2)c([N+](=O)[O-])c1]

15. A pharmaceutical composition comprising a compound of claim 1 and at least one pharmaceutically acceptable excipient.

16. A method of treating a bacterial infection in a subject in need thereof comprising the step of administering a therapeutically effect amount of a compound of claim 1 to the subject.

17. The method of claim 16, wherein the compound has the Formula 3: 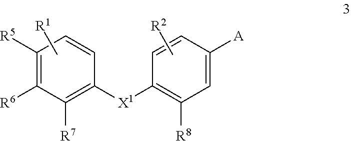
wherein each of R⁵, R⁶, and R⁷ is independently hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, or aralkyl;
R⁸ is hydrogen, nitro, —N(R)₂, —N(R)C(O)R, —N(R)C(O)OR, —N(R)C(O)N(R)₂, —N(R)S(O)₂R, or perhaloalkoxyl; and
A is: 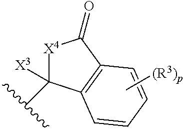
wherein X³ is —OH; and X⁴ is —N(OR⁴)— or —N(R⁴)—; or
A is: 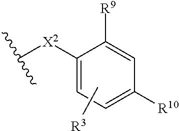
wherein X² is —S—, —O—, —CH₂—, —C(O)—, —C(O)CH₂—*, —(R)C═C(R)—*, —CR₂N(R)—*, or —S(O)₂N(R)—*; each of R⁹ and R¹⁰ is independently selected from the group consisting of hydrogen, halide, nitrile, nitro, azido, —OR, —SR, —N(R)₂, —C(O)R, —C(O)OR, —OC(O)R, —N(R)C(O)R, —C(O)N(R)₂, —N(R)C(O)OR, —OC(O)N(R)—, —OC(O)OR, —N(R)C(O)N(R)₂, —S(O)₂R, —S(O)₂N(R)₂, —N(R)S(O)₂R, perhaloalkoxyl, alkyl, haloalkyl, alkenyl, alkynyl, cycloalkyl, heterocycloalkyl, aryl, heteroaryl, and aralkyl; X² is —S—, —O—, —C(R)₂—, —(R)C═C(R)—*, —CR₂N(R)—*, or —S(O)₂N(R)—*, wherein at least one of R⁹ and R¹⁰ is —C(O)OH and at least one of R⁵, R⁶, and R⁷ is not hydrogen.

18. The method of claim 17, wherein each of R⁵ and R⁶ is independently selected from the group consisting of halide, alkyl, —N(R)C(O)R, and —N(R)₂; each of R⁵ and R⁷ is independently selected from the group consisting of halide, alkyl, —N(R)C(O)R, and —N(R)₂; or R⁷ is —N(R)₂ or alkyl.

19. The method of claim 16, wherein the compound is selected from the group consisting of:

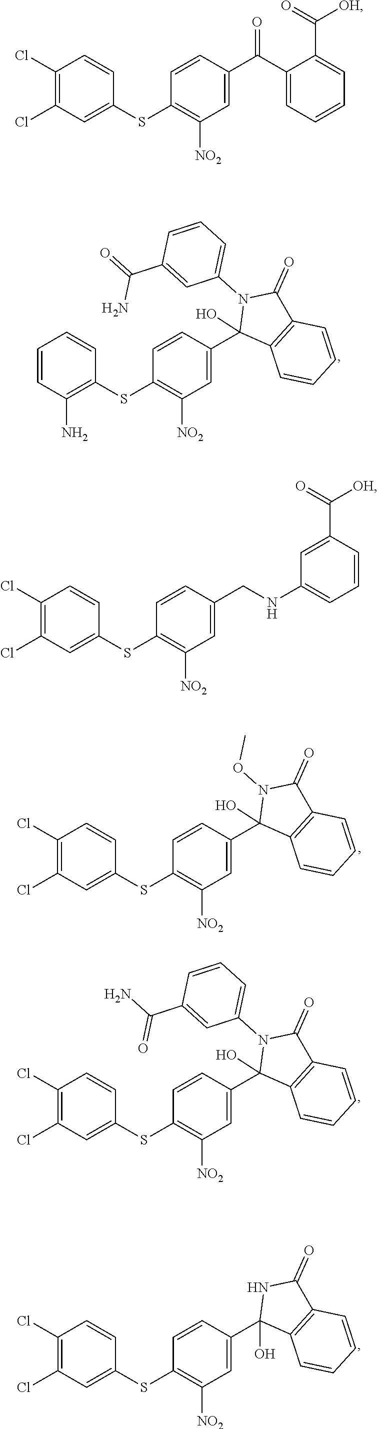

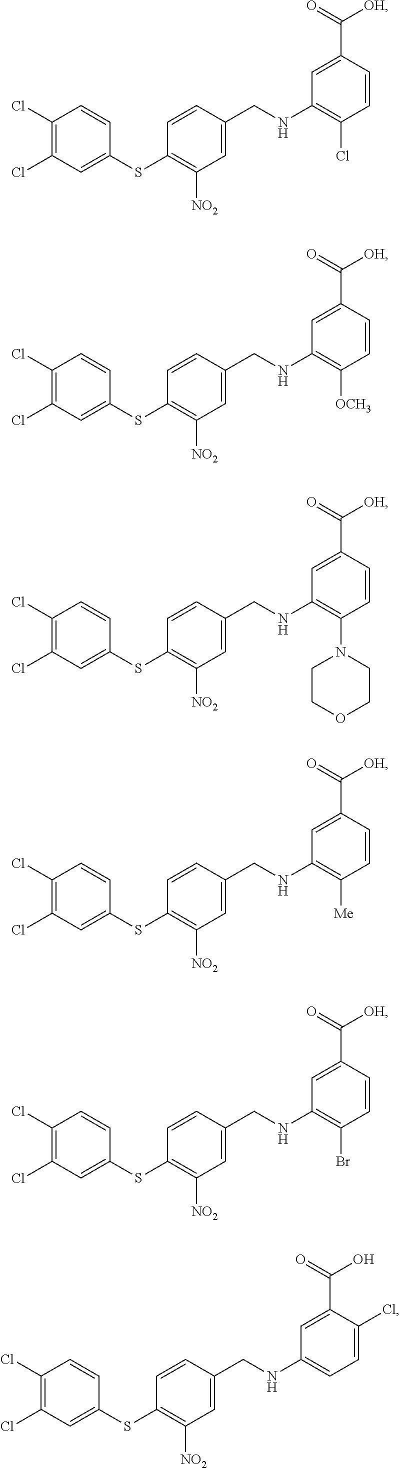 [SMILES: O=C(O)c1ccc(Cl)c(NCc2ccc(Sc3ccc(Cl)c(Cl)c3)c([N+](=O)[O-])c2)c1 ; COc1ccc(C(=O)O)cc1NCc1ccc(Sc2ccc(Cl)c(Cl)c2)c([N+](=O)[O-])c1 ; O=C(O)c1ccc(N2CCOCC2)c(NCc2ccc(Sc3ccc(Cl)c(Cl)c3)c([N+](=O)[O-])c2)c1 ; Cc1ccc(C(=O)O)cc1NCc1ccc(Sc2ccc(Cl)c(Cl)c2)c([N+](=O)[O-])c1 ; O=C(O)c1ccc(Br)c(NCc2ccc(Sc3ccc(Cl)c(Cl)c3)c([N+](=O)[O-])c2)c1 ; O=C(O)c1cc(NCc2ccc(Sc3ccc(Cl)c(Cl)c3)c([N+](=O)[O-])c2)ccc1Cl]

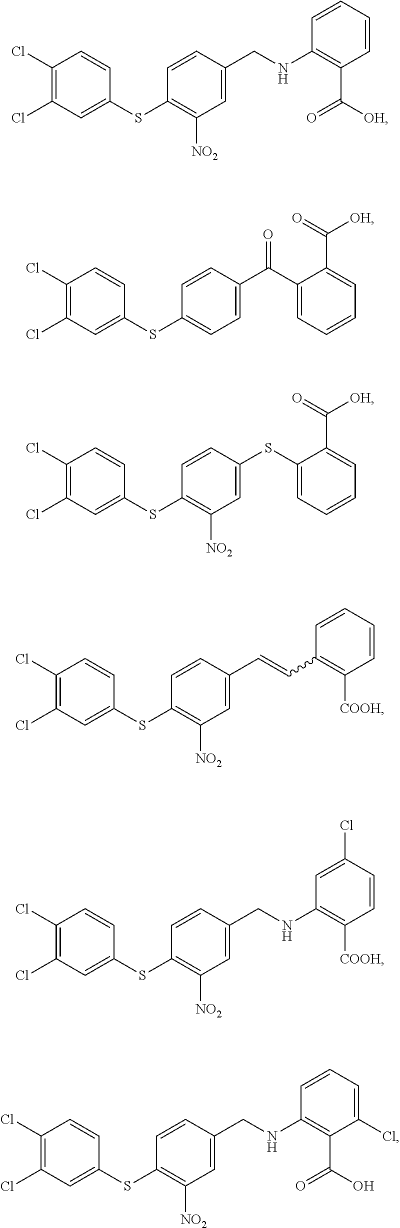 [SMILES: O=C(O)c1ccccc1NCc1ccc(Sc2ccc(Cl)c(Cl)c2)c([N+](=O)[O-])c1 ; O=C(O)c1ccccc1C(=O)c1ccc(Sc2ccc(Cl)c(Cl)c2)cc1 ; O=C(O)c1ccccc1Sc1ccc(Sc2ccc(Cl)c(Cl)c2)c([N+](=O)[O-])c1 ; O=C(O)c1ccccc1C=Cc1ccc(Sc2ccc(Cl)c(Cl)c2)c([N+](=O)[O-])c1 ; O=C(O)c1ccc(Cl)cc1NCc1ccc(Sc2ccc(Cl)c(Cl)c2)c([N+](=O)[O-])c1 ; O=C(O)c1c(Cl)cccc1NCc1ccc(Sc2ccc(Cl)c(Cl)c2)c([N+](=O)[O-])c1]

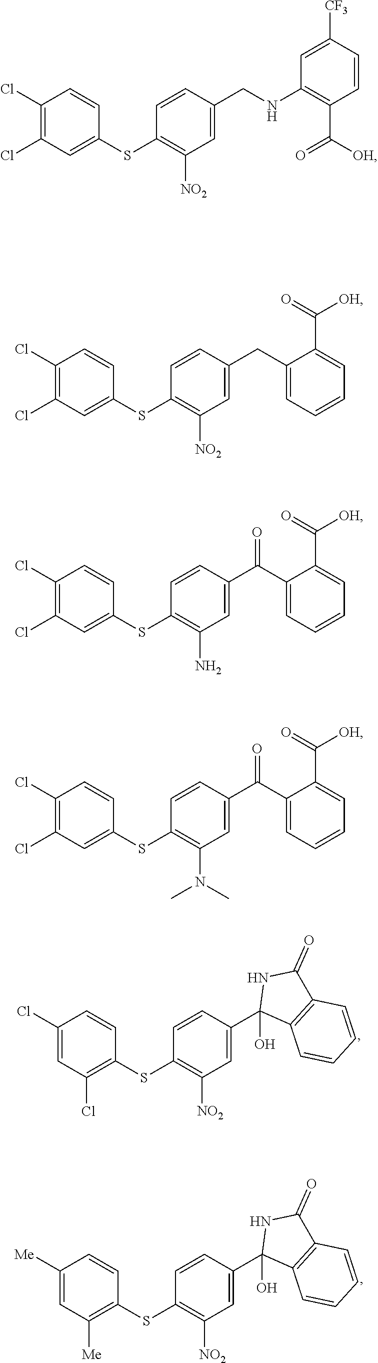 [SMILES: O=C(O)c1ccc(C(F)(F)F)cc1NCc1ccc(Sc2ccc(Cl)c(Cl)c2)c([N+](=O)[O-])c1 ; O=C(O)c1ccccc1Cc1ccc(Sc2ccc(Cl)c(Cl)c2)c([N+](=O)[O-])c1 ; Nc1cc(C(=O)c2ccccc2C(=O)O)ccc1Sc1ccc(Cl)c(Cl)c1 ; CN(C)c1cc(C(=O)c2ccccc2C(=O)O)ccc1Sc1ccc(Cl)c(Cl)c1 ; O=C1NC(O)(c2ccc(Sc3ccc(Cl)cc3Cl)c([N+](=O)[O-])c2)c2ccccc21 ; Cc1ccc(Sc2ccc(C3(O)NC(=O)c4ccccc43)cc2[N+](=O)[O-])c(C)c1]

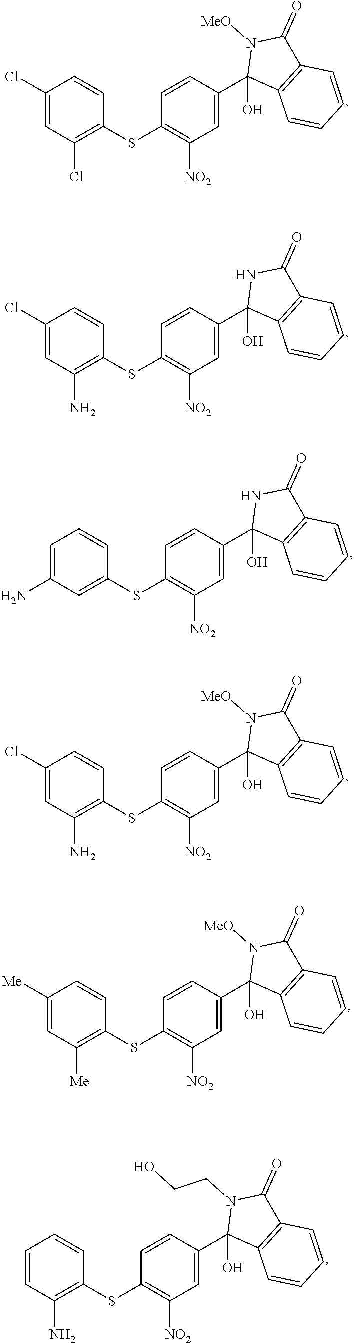 [SMILES: CON1C(=O)c2ccccc2C1(O)c1ccc(Sc2ccc(Cl)cc2Cl)c([N+](=O)[O-])c1 ; Nc1cc(Cl)ccc1Sc1ccc(C2(O)NC(=O)c3ccccc32)cc1[N+](=O)[O-] ; Nc1cccc(Sc2ccc(C3(O)NC(=O)c4ccccc43)cc2[N+](=O)[O-])c1 ; CON1C(=O)c2ccccc2C1(O)c1ccc(Sc2ccc(Cl)cc2N)c([N+](=O)[O-])c1 ; CON1C(=O)c2ccccc2C1(O)c1ccc(Sc2ccc(C)cc2C)c([N+](=O)[O-])c1 ; Nc1ccccc1Sc1ccc(C2(O)c3ccccc3C(=O)N2CCO)cc1[N+](=O)[O-]]

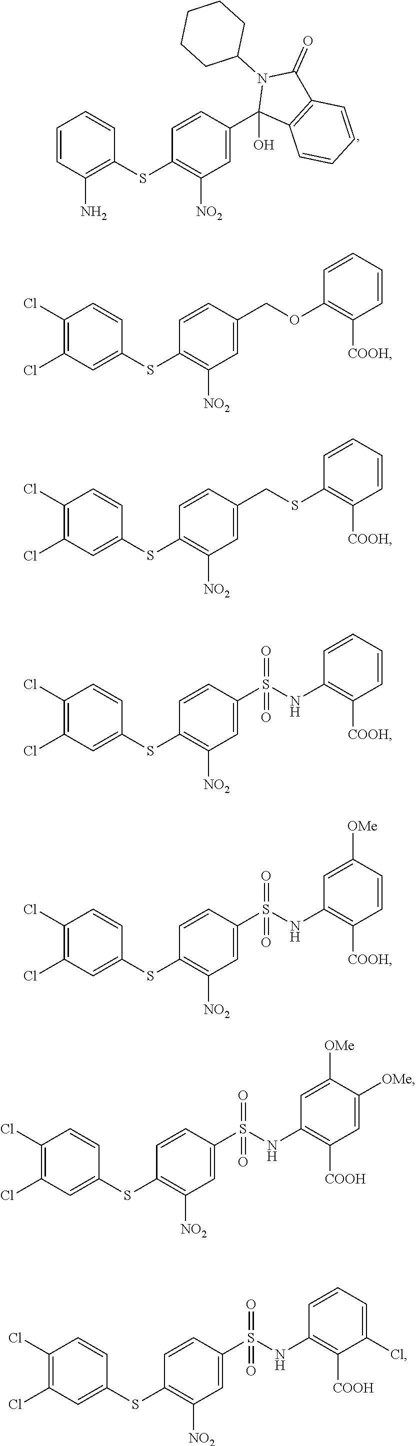

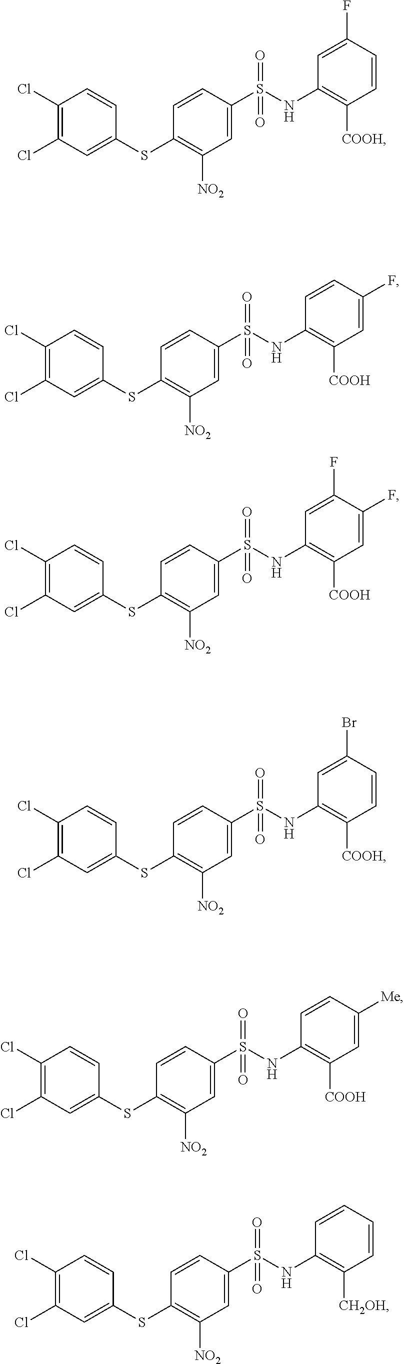 [SMILES: O=C(O)c1ccc(F)cc1NS(=O)(=O)c1ccc(Sc2ccc(Cl)c(Cl)c2)c([N+](=O)[O-])c1 ; O=C(O)c1cc(F)ccc1NS(=O)(=O)c1ccc(Sc2ccc(Cl)c(Cl)c2)c([N+](=O)[O-])c1 ; O=C(O)c1cc(F)c(F)cc1NS(=O)(=O)c1ccc(Sc2ccc(Cl)c(Cl)c2)c([N+](=O)[O-])c1 ; O=C(O)c1ccc(Br)cc1NS(=O)(=O)c1ccc(Sc2ccc(Cl)c(Cl)c2)c([N+](=O)[O-])c1 ; Cc1ccc(NS(=O)(=O)c2ccc(Sc3ccc(Cl)c(Cl)c3)c([N+](=O)[O-])c2)c(C(=O)O)c1 ; O=[N+]([O-])c1cc(S(=O)(=O)Nc2ccccc2CO)ccc1Sc1ccc(Cl)c(Cl)c1]

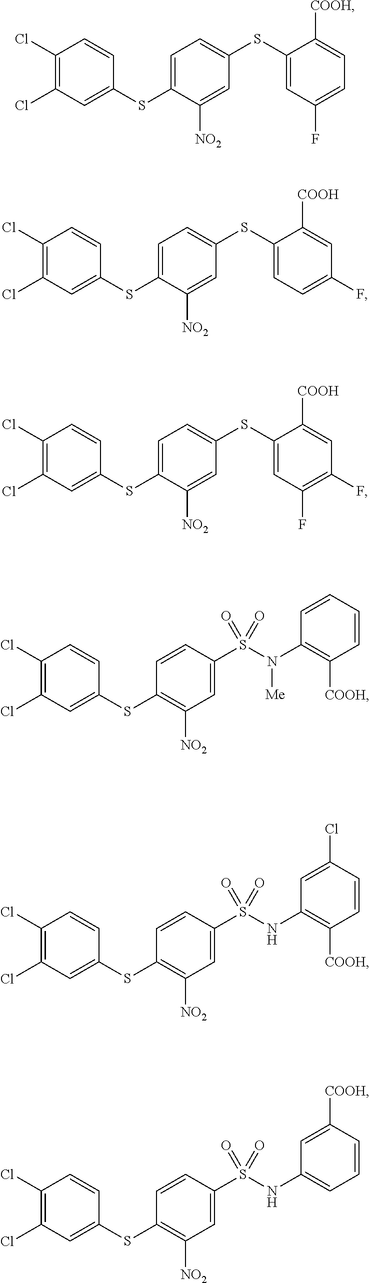 [SMILES: O=C(O)c1ccc(F)cc1Sc1ccc(Sc2ccc(Cl)c(Cl)c2)c([N+](=O)[O-])c1 ; O=C(O)c1cc(F)ccc1Sc1ccc(Sc2ccc(Cl)c(Cl)c2)c([N+](=O)[O-])c1 ; O=C(O)c1cc(F)c(F)cc1Sc1ccc(Sc2ccc(Cl)c(Cl)c2)c([N+](=O)[O-])c1 ; CN(c1ccccc1C(=O)O)S(=O)(=O)c1ccc(Sc2ccc(Cl)c(Cl)c2)c([N+](=O)[O-])c1 ; O=C(O)c1ccc(Cl)cc1NS(=O)(=O)c1ccc(Sc2ccc(Cl)c(Cl)c2)c([N+](=O)[O-])c1 ; O=C(O)c1cccc(NS(=O)(=O)c2ccc(Sc3ccc(Cl)c(Cl)c3)c([N+](=O)[O-])c2)c1]

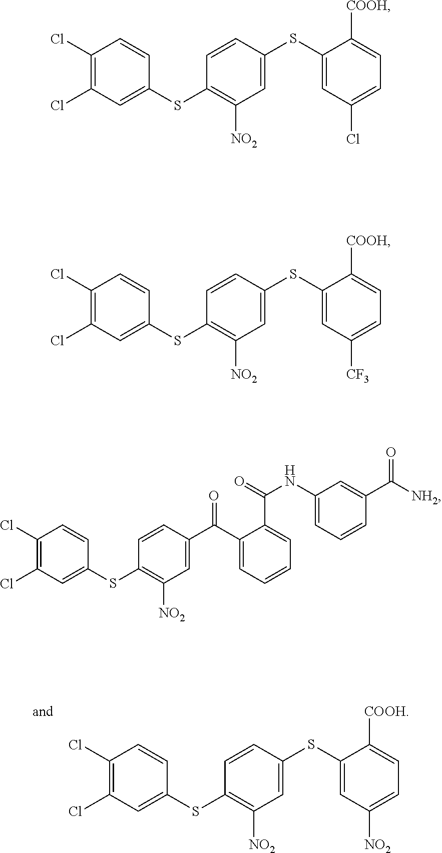 [SMILES: O=C(O)c1ccc(Cl)cc1Sc1ccc(Sc2ccc(Cl)c(Cl)c2)c([N+](=O)[O-])c1 ; O=C(O)c1ccc(C(F)(F)F)cc1Sc1ccc(Sc2ccc(Cl)c(Cl)c2)c([N+](=O)[O-])c1 ; NC(=O)c1cccc(NC(=O)c2ccccc2C(=O)c2ccc(Sc3ccc(Cl)c(Cl)c3)c([N+](=O)[O-])c2)c1 ; O=C(O)c1ccc([N+](=O)[O-])cc1Sc1ccc(Sc2ccc(Cl)c(Cl)c2)c([N+](=O)[O-])c1]

20. The method of claim 16, wherein the bacterial infection comprises Gram-positive bacteria.
